SEQUENCE LISTING (20 sequences)

SEQ ID NO: 1 - RNA, 21 nt, synthetic construct
  source 1..21: mol_type = other RNA
  modified_base 1..2: mod_base = um
  modified_base 3: mod_base = OTHER; note = 2'-O-methyladenosine
  modified_base 4: mod_base = um
  modified_base 5: mod_base = OTHER; note = 2'-O-methyladenosine
  modified_base 6: mod_base = um
  modified_base 7: mod_base = OTHER; note = 2'-fluorocytidine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroadenosine
  modified_base 12..13: mod_base = OTHER; note = 2'-O-methyladenosine
  modified_base 14: mod_base = gm
  modified_base 15: mod_base = OTHER; note = 2'-O-methyladenosine
  modified_base 16: mod_base = gm
  modified_base 17: mod_base = OTHER; note = 2'-O-methyladenosine
  modified_base 18..19: mod_base = um
  modified_base 20..21: mod_base = OTHER; note = 2'-O-methyladenosine
  misc_feature 1..3: note = phosphorothioate backbone
  misc_feature 19..21: note = phosphorothioate backbone
ttatatcagtaaagagattaa

SEQ ID NO: 2 - RNA, 37 nt, synthetic construct
  source 1..37: mol_type = other RNA
  modified_base 1..15: mod_base = OTHER; note = 2'-O-methyl base
  modified_base 16: mod_base = OTHER; note = 2'-fluoro base
  modified_base 17..19: mod_base = OTHER; note = 2'-O-methyl base
  modified_base 20: mod_base = OTHER; note = 2'-fluoro base
  modified_base 21..27: mod_base = OTHER; note = 2'-O-methyl base
  modified_base 28: mod_base = OTHER; note = 2'-fluoro base
  modified_base 29: mod_base = gm
  modified_base 30: mod_base = OTHER; note = 2-fluoro base
  modified_base 31..37: mod_base = OTHER; note = 2'-O-methyl base
  misc_difference 15..17: note = phosphorothioate backbone
  misc_feature 35..37: note = phosphorothioate backbone
gccagggatttccgttaatctctttactgatataatt

SEQ ID NO: 3 - RNA, 26 nt, synthetic construct
  source 1..26: mol_type = other RNA
  modified_base 1: mod_base = cm
  modified_base 2: mod_base = OTHER; note = locked nucleic acid base
  modified_base 3..16: mod_base = OTHER; note = 2'-O-methyl base
  modified_base 17: mod_base = OTHER; note = locked nucleic acid base
  modified_base 18: mod_base = gm
  modified_base 19: mod_base = OTHER; note = locked nucleic acid base
  modified_base 20: mod_base = gm
  modified_base 21: mod_base = OTHER; note = locked nucleic acid base
  modified_base 22..26: mod_base = OTHER; note = 2'-O-methyl base
  misc_feature 1..2: note = phosphorothioate backbone
  misc_feature 14..26: note = phosphorothioate backbone
  misc_binding 26: bound_moiety = 3' Amino Modifier C6 dT
cggaaatccctggcaatgtgattttt

SEQ ID NO: 4 - RNA, 30 nt, synthetic construct
  source 1..30: mol_type = other RNA
  modified_base 1: mod_base = cm
  modified_base 2: mod_base = OTHER; note = locked nucleic acid base
  modified_base 3..16: mod_base = OTHER; note = 2'-O-methyl base
  modified_base 17: mod_base = OTHER; note = locked nucleic acid base T
  modified_base 18: mod_base = gm
  modified_base 19: mod_base = OTHER; note = locked nucleic acid base T
  modified_base 20: mod_base = gm
  modified_base 21: mod_base = OTHER; note = locked nucleic acid base
  modified_base 22..30: mod_base = OTHER; note = 2'-O-methyl base
  misc_feature 1..2: note = phosphorothioate backbone
  misc_feature 14..30: note = phosphorothioate backbone
  misc_binding 30: bound_moiety = 3' amino modifier C6 dT
cggaaatccctggcaatgtgattttattta

SEQ ID NO: 5 - RNA, 22 nt, synthetic construct
  source 1..22: mol_type = other RNA
  modified_base 1: mod_base = cm
  modified_base 2: mod_base = gm
  modified_base 3: mod_base = OTHER; note = locked nucleic acid base
  modified_base 4: mod_base = OTHER; note = 2'-O-methyl base
  modified_base 5: mod_base = OTHER; note = locked nucleic acid base
  modified_base 6..7: mod_base = OTHER; note = 2'-O-methyl base
  modified_base 8: mod_base = OTHER; note = locked nucleic acid base
  modified_base 9..10: mod_base = cm
  modified_base 11: mod_base = OTHER; note = locked nucleic acid base T
  modified_base 12: mod_base = gm
  modified_base 13: mod_base = OTHER; note = locked nucleic acid base
  modified_base 14..16: mod_base = OTHER; note = 2'-O-methyl base
  modified_base 17: mod_base = OTHER; note = locked nucleic acid base T
  modified_base 18: mod_base = gm
  modified_base 19: mod_base = OTHER; note = locked nucleic acid base T
  modified_base 20: mod_base = gm
  modified_base 21: mod_base = OTHER; note = locked nucleic acid base
  modified_base 22: mod_base = OTHER; note = locked nucleic acid base T
  misc_feature 1..3: note = phosphorothioate backbone
  misc_feature 12..22: note = phosphorothioate backbone
  misc_binding 1: bound_moiety = 5' triethylene glycol spacer
cggaaatccctggcaatgtgat

SEQ ID NO: 6 - RNA, 22 nt, synthetic construct
  source 1..22: mol_type = other RNA
  modified_base 1: mod_base = cm
  modified_base 2: mod_base = gm
  modified_base 3: mod_base = OTHER; note = locked nucleic acid base
  modified_base 4: mod_base = OTHER; note = 2'-O-methyl base
  modified_base 5: mod_base = OTHER; note = locked nucleic acid base
  modified_base 6..7: mod_base = OTHER; note = 2'-O-methyl base
  modified_base 8: mod_base = OTHER; note = locked nucleic acid base
  modified_base 9..10: mod_base = cm
  modified_base 11: mod_base = OTHER; note = locked nucleic acid base T
  modified_base 12: mod_base = gm
  modified_base 13: mod_base = OTHER; note = locked nucleic acid base
  modified_base 14..16: mod_base = OTHER; note = 2'-O-methyl base
  modified_base 14: mod_base = OTHER; note = locked nucleic acid base T
  modified_base 18: mod_base = gm
  modified_base 17: mod_base = OTHER; note = locked nucleic acid base T
  modified_base 19: mod_base = OTHER; note = locked nucleic acid base T
  modified_base 20: mod_base = gm
  modified_base 21: mod_base = OTHER; note = locked nucleic acid base
  modified_base 22: mod_base = OTHER; note = locked nucleic acid base T
  misc_feature 1..3: note = phosphorothioate backbone
  misc_feature 12..22: note = phosphorothioate backbone
  misc_binding 1: bound_moiety = 5' triethylene glycol spacer
  misc_binding 22: bound_moiety = palmitic acid
cggaaatccctggcaatgtgat

SEQ ID NO: 7 - RNA, 22 nt, synthetic construct
  source 1..22: mol_type = other RNA
  modified_base 2: mod_base = OTHER; note = locked nucleic acid base
  modified_base 3..7: mod_base = OTHER; note = 2'-O-methyl base
  modified_base 8: mod_base = OTHER; note = locked nucleic acid base
  modified_base 9: mod_base = OTHER; note = 2'-O-methyladenosine
  modified_base 10: mod_base = gm
  modified_base 11: mod_base = OTHER; note = locked nucleic base T
  modified_base 12..15: mod_base = OTHER; note = 2'-O-methyl base
  modified_base 16: mod_base = OTHER; note = locked nucleic acid base T
  modified_base 17: mod_base = OTHER; note = 2'-O-methyladenosine
  modified_base 18: mod_base = OTHER; note = locked nucleic acid base
  modified_base 19: mod_base = gm
  modified_base 20: mod_base = cm
  modified_base 21: mod_base = OTHER; note = locked nucleic acid base T
  modified_base 22: mod_base = OTHER; note = 2'-O-methyladenosine
  misc_feature 1..2: note = phosphorothioate backbone
  misc_feature 12..22: note = phosphorothioate backbone
  misc_binding 22: bound_moiety = 3' amino modifier C6 dT
  modified_base 1: mod_base = OTHER; note = 2'-O-methyl base
tcaacatcagtctgataagcta

SEQ ID NO: 8 - RNA, 35 nt, synthetic construct
  source 1..35: mol_type = other RNA
  misc_feature 13..15: note = phosphorothioate backbone
  modified_base 14: mod_base = OTHER; note = 2'-fluoro base
  modified_base 15..17: mod_base = OTHER; note = 2'-O-methyl base
  modified_base 18: mod_base = OTHER; note = 2'-fluoro base
  modified_base 19..25: mod_base = OTHER; note = 2'-O-methyl base
  modified_base 26: mod_base = OTHER; note = 2'-fluoro base
  modified_base 27: mod_base = gm
  modified_base 28: mod_base = OTHER; note = 2'-fluoro base
  modified_base 29..35: mod_base = OTHER; note = 2'-O-methyl base
  misc_feature 33..35: note = phosphorothioate backbone
  modified_base 1..13: mod_base = OTHER; note = 2'-O-methyl base
gactgatgttgattaatctctttactgatataatt

SEQ ID NO: 9 - RNA, 22 nt, synthetic construct
  source 1..22: mol_type = other RNA
  modified_base 1: mod_base = OTHER; note = 2'-O-methyl base
  modified_base 2: mod_base = OTHER; note = locked nucleic acid base
  modified_base 3..7: mod_base = OTHER; note = 2'-O-methyl base
  modified_base 8: mod_base = OTHER; note = locked nucleic acid base
  modified_base 9: mod_base = OTHER; note = 2'-O-methyladenosine
  modified_base 10: mod_base = gm
  modified_base 11: mod_base = OTHER; note = locked nucleic acid base T
  modified_base 12..15: mod_base = OTHER; note = 2'-O-methyl base
  modified_base 16: mod_base = OTHER; note = locked nucleic acid base T
  modified_base 17: mod_base = OTHER; note = 2'-O-methyladenosine
  modified_base 18: mod_base = OTHER; note = locked nucleic acid base
  modified_base 19: mod_base = gm
  modified_base 20: mod_base = cm
  modified_base 21: mod_base = OTHER; note = locked nucleic acid base T
  modified_base 22: mod_base = OTHER; note = 2'-O-methyladenosine
  misc_structure 1..2: note = phosphorothioate backbone
  misc_feature 14..22: note = phosphorothioate backbone
  misc_binding 22: bound_moiety = 3' amino modifier C6 dT
tcaacatcagtctgataagcta

SEQ ID NO: 10 - RNA, 37 nt, synthetic construct
  source 1..37: mol_type = other RNA
  modified_base 16: mod_base = OTHER; note = 2'-fluoro base
  misc_feature 15..17: note = phosphorothioate backbone
  modified_base 17..19: mod_base = OTHER; note = 2'-O-methyl base
  modified_base 20: mod_base = OTHER; note = 2'-fluoro base
  modified_base 21..27: mod_base = OTHER; note = 2'-O-methyl base
  modified_base 28: mod_base = OTHER; note = 2'-fluoro base
  modified_base 29: mod_base = gm
  modified_base 30: mod_base = OTHER; note = 2-fluoro base
  modified_base 31..37: mod_base = OTHER; note = 2'-O-methyl base
  misc_feature 35..37: note = phosphorothioate backbone
  modified_base 1..15: mod_base = OTHER; note = 2'-O-methyl base
cagactgatgttgattaatctctttactgatataatt

SEQ ID NO: 11 - RNA, 22 nt, synthetic construct
  source 1..22: mol_type = other RNA
  modified_base 1..4: mod_base = OTHER; note = 2'-O-methyl base
  modified_base 5: mod_base = OTHER; note = locked nucleic acid base
  modified_base 6..10: mod_base = OTHER; note = 2'-O-methyl base
  modified_base 11: mod_base = OTHER; note = locked nucleic acid base
  modified_base 12..15: mod_base = OTHER; note = 2'-O-methyl base
  misc_feature 12..22: note = phosphorothioate backbone
  modified_base 16: mod_base = OTHER; note = locked nucleci acid base
  modified_base 17: mod_base = gm
  modified_base 18: mod_base = OTHER; note = 2'-O-methyl base
  modified_base 19: mod_base = OTHER; note = locked nucleic acid base
  modified_base 20..22: mod_base = OTHER; note = 2'-O-methyl base
  misc_binding 22: bound_moiety = 3' amino modifier C6 dT
  misc_feature 1..2: note = phosphorothioate backbone
taaccgatttcaaatggtgcta

SEQ ID NO: 12 - RNA, 35 nt, synthetic construct
  source 1..35: mol_type = other RNA
  misc_structure 13..15: note = phosphorothioate backbone
  modified_base 14: mod_base = OTHER; note = 2'-fluoro base
  modified_base 15..17: mod_base = OTHER; note = 2'-O-methyl base
  modified_base 18: mod_base = OTHER; note = 2'-fluoro base
  modified_base 19..25: mod_base = OTHER; note = 2'-O-methyl base
  modified_base 26: mod_base = OTHER; note = 2'-fluoro base
  modified_base 27: mod_base = gm
  modified_base 28: mod_base = OTHER; note = 2'-fluoro base
  modified_base 29..35: mod_base = OTHER; note = 2'-O-methyl base
  misc_feature 33..35: note = phosphorothioate backbone
  modified_base 1..13: mod_base = OTHER; note = 2'-O-methyl base
tgaaatcggttattaatctctttactgatataatt

SEQ ID NO: 13 - RNA, 26 nt, synthetic construct
  source 1..26: mol_type = other RNA
  modified_base 1..4: mod_base = OTHER; note = 2'-O-methyl base
  modified_base 5: mod_base = OTHER; note = locked nucleic acid base
  modified_base 6..18: mod_base = OTHER; note = 2'-O-methyl base
  modified_base 19: mod_base = OTHER; note = locked nucleic acid base T
  modified_base 20..24: mod_base = OTHER; note = 2'-O-methyl base
  modified_base 25: mod_base = OTHER; note = locked nucleic acid base T
  modified_base 26: mod_base = cm
  misc_feature 14..26: note = phosphorothioate backbone
  misc_binding 26: bound_moiety = 3' amino modifier C6 dT
  misc_structure 1..2: note = phosphorothioate backbone
agccgagtgatccaccgctaagagtc

SEQ ID NO: 14 - RNA, 37 nt, synthetic construct
  source 1..37: mol_type = other RNA
  misc_feature 15..17: note = phosphorothioate backbone
  modified_base 16: mod_base = OTHER; note = 2'-fluoro base
  modified_base 17..19: mod_base = OTHER; note = 2'-O-methyl base
  modified_base 20: mod_base = OTHER; note = 2'-fluoro base
  modified_base 21..27: mod_base = OTHER; note = 2'-O-methyl base
  modified_base 28: mod_base = OTHER; note = 2'-fluoro base
  modified_base 29: mod_base = gm
  modified_base 30: mod_base = OTHER; note = 2'-fluoro base
  modified_base 31..37: mod_base = OTHER; note = 2'-O-methyl base
  misc_feature 35..37: note = phosphorothioate backbone
  modified_base 1..15: mod_base = OTHER; note = 2'-O-methyl base
tggatcactcggctttaatctctttactgatataatt

SEQ ID NO: 15 - RNA, 24 nt, synthetic construct
  source 1..24: mol_type = other RNA
  modified_base 1..4: mod_base = OTHER; note = 2'-O-methyl base
  modified_base 5: mod_base = OTHER; note = locked nucleic acid base
  modified_base 6..21: mod_base = OTHER; note = 2'-O-methyl base
  modified_base 12: mod_base = OTHER; note = locked nucleic acid base
  modified_base 23: mod_base = cm
  modified_base 24: mod_base = OTHER; note = 2'-O-methyl U
  misc_feature 12..24: map = phosphorothioate backbone
  misc_feature 1..2: note = phosphorothioate backbone
  misc_binding 24: bound_moiety = 3' amino modifier C6 dT
gcacgagccgagtgatccaccgct

SEQ ID NO: 16 - RNA, 35 nt, synthetic construct
  source 1..35: mol_type = other RNA
  misc_feature 13..15: note = phosphorothioate backbone
  modified_base 14: mod_base = OTHER; note = 2'-fluoro base
  modified_base 15..17: mod_base = OTHER; note = 2'-O-methyl base
  modified_base 18: mod_base = OTHER; note = 2'-fluoro base
  modified_base 19..25: mod_base = OTHER; note = 2'-O-methyl base
  modified_base 26: mod_base = OTHER; note = 2'-fluoro base
  modified_base 27: mod_base = gm
  modified_base 28: mod_base = OTHER; note = 2'-fluoro base
  modified_base 29..35: mod_base = OTHER; note = 2'-O-methyl base
  misc_feature 33..35: note = phosphorothioate backbone
  modified_base 1..13: mod_base = OTHER; note = 2'-O-methyl base
ctcggctcgtgcttaatctctttactgatataatt

SEQ ID NO: 17 - RNA, 22 nt, synthetic construct
  source 1..22: mol_type = other RNA
  misc_feature 1: note = The 5' terminus is attached to a triethylene glycol spacer va a phosphorothioate backbone connection.
  misc_feature 22: note = 3' terminus is attached to an amino modifier C6 dT via a phosphorothioate backbone connection.
  misc_feature 1..3: note = phosphorothioate backbone
  misc_feature 12..22: note = phosphorothioate backbone
  modified_base 1: mod_base = cm
  modified_base 2: mod_base = gm
  modified_base 3: mod_base = OTHER; note = locked nucleci acid base
  modified_base 4: mod_base = OTHER; note = 2'-O-methyladenosine
  modified_base 5: mod_base = OTHER; note = locked nucleic acid base
  modified_base 6..7: mod_base = OTHER; note = 2'-O-methyl base
  modified_base 8: mod_base = OTHER; note = locked nucleic acid base
  modified_base 9..10: mod_base = cm
  modified_base 11: mod_base = OTHER; note = locked nucleic acid base T
  modified_base 12: mod_base = gm
  modified_base 13: mod_base = OTHER; note = locked nucleic acid base
  modified_base 14: mod_base = cm
  modified_base 15..16: mod_base = OTHER; note = 2'-O-methyladenosine
  modified_base 17: mod_base = OTHER; note = locked nucleic acid base T
  modified_base 18: mod_base = gm
  modified_base 19: mod_base = OTHER; note = locked nucleic acid base T
  modified_base 20: mod_base = gm
  modified_base 21: mod_base = OTHER; note = locked nucleic acid base A
  modified_base 22: mod_base = OTHER; note = locked nucleic acid base T
cggaaatccctggcaatgtgat

SEQ ID NO: 18 - RNA, 32 nt, synthetic construct
  source 1..32: mol_type = other RNA
  misc_feature 32: note = The 3' terminus is attached to an amino modifier C6 dT via a phosphorothioate backbone connection.
  misc_feature 1..3: note = phosphorothioate backbone
  modified_base 1: mod_base = cm
  modified_base 2: mod_base = OTHER; note = locked nucleic acid base T
  modified_base 3..4: mod_base = gm
  modified_base 5: mod_base = OTHER; note = locked nucleic acid base
  modified_base 6..8: mod_base = OTHER; note = 2'-O-methyladenosine
  modified_base 9: mod_base = gm
  modified_base 10: mod_base = OTHER; note = locked nucleic acid base
  modified_base 11..12: mod_base = gm
  modified_base 13: mod_base = OTHER; note = 2'-O-methyl base
  modified_base 14: mod_base = OTHER; note = locked nucleic acid base
  modified_base 15..16: mod_base = OTHER; note = 2'-O-methyl base
  modified_base 17: mod_base = OTHER; note = locked nucleic acid base T
  modified_base 18: mod_base = gm
  modified_base 19: mod_base = OTHER; note = locked nucleic acid base
  modified_base 20..26: mod_base = OTHER; note = 2'-O-methyl base
  modified_base 27: mod_base = OTHER; note = locked nucleic acid base
  modified_base 28..30: mod_base = OTHER; note = 2'-methyl base
  modified_base 31: mod_base = OTHER; note = locked nucleic acid T
  modified_base 32: mod_base = OTHER; note = locked nucleic acid C
  misc_feature 20..32: note = phosphorothioate backbone
  misc_feature 1: note = The 5' terminus is attached to triethylene glycol spacer via a phosphorothioate backbone connection.
ctggcaaagcggtcattgagctccatcatctc

SEQ ID NO: 19 - RNA, 33 nt, synthetic construct
  source 1..33: mol_type = other RNA
  modified_base 1..13: mod_base = OTHER; note = 2'-O-methyl base
  misc_feature 13..15: note = phosphorothioate backbone
  modified_base 14: mod_base = OTHER; note = 2'-fluoro base
  modified_base 15..17: mod_base = OTHER; note = 2'-O-methyl base
  modified_base 18: mod_base = OTHER; note = 2'-fluoro base
  modified_base 19..25: mod_base = OTHER; note = 2'-O-methyl base
  modified_base 26: mod_base = OTHER; note = 2'-fluoro base
  modified_base 27: mod_base = gm
  modified_base 28: mod_base = OTHER; note = 2'-fluoro base
  modified_base 29..33: mod_base = OTHER; note = 2'-O-methyl base
  misc_feature 33: note = The 3' terminus is attached to a C3 spacer via a phosphorothioate backbone connection, which is then joined to SEQ ID NO: 20.
cgctttgccagattaatctctttactgatataa

SEQ ID NO: 20 - RNA, 11 nt, synthetic construct
  source 1..11: mol_type = other RNA
  modified_base 1..11: mod_base = OTHER; note = 2'-O-methyl base
  misc_feature 1..2: note = phosphorothioate backbone
agctcaatgac

(19) United States
(12) Patent Application Publication
(10) Pub. No.: US 2024/0409933 A1
(43) Pub. Date: Dec. 12, 2024

(54) CONDITION-ACTIVATABLE NUCLEIC ACID CONSTRUCTS AND THEIR USES FOR TREATING NEUROLOGICAL DISEASES

(71) Applicant: SWITCH THERAPEUTICS INC., South San Francisco, CA (US)

(72) Inventors: Si-ping Han, South San Francisco, CA (US); Deepshikha Datta, South San Francisco, CA (US); Marianna Kiraly, South San Francisco, CA (US)

(21) Appl. No.: 18/702,733

(22) PCT Filed: Oct. 20, 2022
(86) PCT No.: PCT/US2022/078466

§ 371 Date: Apr. 18, 2024

Related U.S. Application Data
(60) Provisional application No. 63/270,189, filed on Oct. 21, 2021.
(60) Provisional application No. 63/332,099, filed on Apr. 18, 2022.

(51) Int. Cl. C12N 15/113; A61K 9/00; A61P 25/28

(52) U.S. Cl. CPC C12N 15/113; A61K 9/0019; A61K 9/0085; A61P 25/28; C12N 2310/14; C12N 2310/315; C12N 2310/321; C12N 2310/322; C12N 2310/3515; C12N 2310/3519; C12N 2320/32

(57) ABSTRACT

Provided herein include methods, compositions, and kits suitable for use in preventing and treating a neurological disease or disorder. The method can comprise administering to a subject in need thereof a composition comprising conditionally activatable small interfering RNA (siRNA) complexes.

### RELATED APPLICATIONS

The present application is a National Stage application of PCT Application PCT/US2022/078466, filed on Oct. 20, 2022, which claims priority under 35 U.S.C. § 119 (e) to U.S. Provisional Application No. 63/270,189, filed on Oct. 21, 2021, and U.S. Provisional Application No. 63/332,099, filed on Apr. 18, 2022, the contents of which are herein expressly incorporated by reference in their entireties.

### REFERENCE TO SEQUENCE LISTING

The present application is being filed along with a Sequence Listing in electronic format. The Sequence Listing is provided as a file entitled 75EN-329794-WO_SeqListing, created Jul. 13, 2022, which is 151 kilobytes in size. The information in the electronic format of the Sequence Listing is incorporated herein by reference in its entirety.

### BACKGROUND

### Field

The present disclosure relates generally to the field of nucleic acid, for example, conditionally activatable small interfering RNA complexes.

### Description of the Related Art

Despite emerging developments in the field of dynamic nucleic acid nanotechnology and biomolecular computing, there is still a challenge to develop targeted RNAi therapy that can use nucleic acid logic switches to sense RNA transcripts (such as mRNAs and miRNAs), thereby restricting RNA interfering (RNAi) therapy to specific populations of disease-related cells. In particular, there is a need to develop targeted and conditionally activated RNAi therapy with improved drug potency, sensitivity, and stability, low design complexity, and low dosage requirement to treat diseases and disorders.

### SUMMARY

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

Disclosed herein includes a method of modulating a target RNA in the nervous system of a subject. The method, in some embodiments, comprises: administering a nucleic acid complex to the subject, the nucleic acid complex, comprising: a first nucleic acid duplex formed by a second nucleic acid strand binding to a portion of a first nucleic acid strand, wherein the first nucleic acid duplex comprises a sequence complementary to a target RNA, and a second nucleic acid duplex formed by a third nucleic acid strand binding to at least one portion of the first nucleic acid strand, wherein the third nucleic acid strand comprises an overhang, wherein the overhang is not complementary to the first nucleic acid strand and is capable of binding to an input nucleic acid strand to cause the displacement of the third nucleic acid strand from the first nucleic acid strand; thereby distributing the nucleic acid complex into one or more regions of the nervous system of the subject, wherein the input nucleic acid strand binds to the overhang of the third nucleic acid strand in the one or more regions of the nervous system to cause displacement of the third nucleic acid strand from the first nucleic acid strand to release the sequence complementary to the target RNA, thereby reducing the activity of the target RNA or protein expression from the target RNA by at least 30% in the subject to treat the neurological disease or disorder. Also disclosed herein includes a method of treating a neurological disease or disorder, comprising: administering a nucleic acid complex to a subject in need thereof, the nucleic acid complex, comprising: a first nucleic acid duplex formed by a second nucleic acid strand binding to a portion of a first nucleic acid strand, wherein the first nucleic acid duplex comprises a sequence complementary to a target RNA, and a second nucleic acid duplex formed by a third nucleic acid strand binding to at least one portion of the first nucleic acid strand, wherein the third nucleic acid strand comprises an overhang, wherein the overhang is not complementary to the first nucleic acid strand and is capable of binding to an input nucleic acid strand to cause the displacement of the third nucleic acid strand from the first nucleic acid strand; thereby distributing the nucleic acid complex into one or more regions of the nervous system of the subject, wherein the input nucleic acid strand binds to the overhang of the third nucleic acid strand in the one or more regions of the nervous system to cause displacement of the third nucleic acid strand from the first nucleic acid strand to release the sequence complementary to a target RNA related to the neurological disease or disorder, thereby reducing the activity of the target RNA or protein expression from the target RNA by at least 30% in the subject to treat the neurological disease or disorder.

The target RNA can be, for example, a nervous system-specific RNA. In some embodiments, the portion of the first nucleic acid strand that is bound to the second nucleic acid comprises a sequence complementary to the target RNA. The sequence complementary to the target RNA can be 10-35 nucleosides in length, and in some embodiments, 10-21 nucleotides in length. In some embodiments, the second nucleic acid does not have: (i) a 3′ overhang in the first nucleic acid duplex and/or (ii) a terminal moiety attached on the 5′ terminus, and optionally wherein the terminal moiety is a blocker.

In some embodiments, the nucleic acid complex comprises: the first nucleic acid strand comprising 20-70 linked nucleosides; the second nucleic acid strand binding to a central region of the first nucleic acid strand to form the first nucleic acid duplex; and the third nucleic acid strand binding to a 5′ region and a 3′ region of the first nucleic acid strand to form the second nucleic acid duplex.

In some embodiments, the central region of the first nucleic acid strand comprises the sequence complementary to the target RNA. In some embodiments, the central region of the first nucleic acid strand is linked to the 5′ region of the first nucleic acid strand via a 5′ connector, the central region of the first nucleic acid strand is linked to the 3′ region of the first nucleic acid strand via a 3′ connector, or both.

In some embodiments, the 5′ connector, the 3′ connector, or both comprise a C3 3-carbon linker, a nucleotide, a modified nucleotide, or a exonuclease cleavage-resistant moiety, or a combination thereof. The modified nucleotide can be, for example, a 2′-O-methyl nucleotide or a 2′-F nucleotide.

In some embodiments, the second nucleic acid strand is fully complementary to the central region of the first nucleic acid strand, thereby forming blunt ends at the 5′ and 3′ termini of the second nucleic acid strand in the first nucleic acid duplex. In some embodiments, the nucleic acid complex comprises: the first nucleic acid strand comprising 20-60 linked nucleosides; the second nucleic acid strand binding to a first region of the first nucleic acid strand to form the first nucleic acid duplex; the third nucleic acid strand binding to a second region of the first nucleic acid strand to form the second nucleic acid duplex; and wherein the first region of the first nucleic acid strand is 3′ of the second region of the first nucleic acid strand, and the third nucleic acid strand does not bind to any region of the first nucleic acid strand that is 3′ of the first region of the first nucleic acid strand.

In some embodiments, the second nucleic acid strand binds to 17-22 linked nucleotides in the first region of the first nucleic acid strand to form the first nucleic acid duplex. In some embodiments, the third nucleic acid strand binds to about 14 linked nucleotides in the second region of the first nucleic acid strand to form the second nucleic acid duplex. In some embodiments, the first region of the first nucleic acid strand is linked to the second region of the first nucleic acid strand via a linker, optionally the linker comprises a C3 3-carbon linker, a nucleotide, a modified nucleotide, or a exonuclease cleavage-resistant moiety, or a combination thereof. In some embodiments, the first nucleic acid strand comprises a 3′ overhang in the first nucleic acid duplex, optionally the 3′ overhang of the first nucleic acid is one, two, or three nucleotides in length.

In some embodiments, the second nucleic acid strand does not have an overhang at 3′ terminus, or 5′ terminus, or both in the first nucleic acid duplex. In some embodiments, the 5′ terminus of the second nucleic acid strand comprises a blocking moiety. In some embodiments, the overhang of the third nucleic acid strand is capable of binding to the input nucleic acid strand to form a toehold, thereby causing the displacement of the second nucleic acid strand from the first nucleic acid strand; optionally the overhang of the third nucleic acid strand is at the 3′ terminus of the third nucleic acid strand, the overhang of the third nucleic acid strand is 8-16 nucleotides in length, or both.

The 5′ terminus, the 3′ terminus, or both of the third nucleic acid strand can comprise a terminal moiety. In some embodiments, the terminal moiety comprises a ligand, a fluorophore, a exonuclease, a fatty acid, a Cy3, an inverted dT attached to a tri-ethylene glycol, or a combination thereof. In some embodiments, the terminal moiety is a palmitic acid, for example, a palmitic acid attached to the 3′ terminus of the third nucleic acid strand.

In some embodiments, at least 80%, at least 85%, at least 90%, at least 95%, or all of the nucleosides of one or more of the first nucleic acid strand, the second nucleic acid strand and the third nucleic acid strand are chemically modified. In some embodiments, the first nucleic acid duplex does not comprise a Dicer cleavage site. In some embodiments, the nucleic acid duplex does not comprise a Dicer cleavage site. In some embodiments, the one or more regions of the nervous systems comprises a central nervous system (CNS), a peripheral nervous system (PNS) or both. In some embodiments, the one or more regions of the nervous system comprises: right cortex, prefrontal cortex, sensory cortex, visual cortex, striatum, dorsal hippocampus, ventral hippocampus, thalamus, cerebellum, midbrain, left hemisphere, right hemisphere, dorsal root ganglia, or a combination thereof.

In some embodiments, the neurological disease or disorder is a CNS disease or disorder, and optionally wherein the CNS disease or disorder is selected from the group consisting of: Adrenoleukodystrophy, Alzheimer disease, Amyotrophic lateral sclerosis, Angelman syndrome, Ataxia telangiectasia, Charcot-Marie-Tooth syndrome, Cockayne syndrome, Deafness, Duchenne muscular dystrophy, Epilepsy, Essential tremor, Fragile X syndrome, Friedreich's ataxia, Gaucher disease, Huntington disease, Lesch-Nyhan syndrome, Maple syrup urine disease, Menkes syndrome, Myotonic dystrophy, Narcolepsy, Neurofibromatosis, Niemann-Pick disease, Parkinson disease, Phenylketonuria, Prader-Willi syndrome, Refsum disease, Rett syndrome, Spinal muscular atrophy, Spinocerebellar ataxia, Tangier disease, Tay-Sachs disease, Tuberous sclerosis, Von Hippel-Lindau syndrome, Williams syndrome, Wilson's disease, and Zellweger syndrome. In some embodiments, the neurological disease or disorder is a CNS disease or disorder; and optionally wherein the PNS disease or disorder is selected from the group consisting of: Acute motor axonal neuropathy; Botulism; Charcot-Marie-Tooth disease types 1A, 1B and 1X; Cisplatin neuropathy; Diabetic neuropathy; Diphtheritic neuropathy; Familial amyloid neuropathy; Guillain-Barré syndrome; Lambert-Eaton syndrome; Leprosy; Neuropathy with IgM1 anti-myelin-associated glycoprotein; Pyridoxine neuropathy; and Refsum's disease.

In some embodiments, the nucleic acid complex is distributed in all of the right cortex, prefrontal cortex, sensory cortex, visual cortex, striatum, dorsal hippocampus, ventral hippocampus, thalamus, cerebellum, midbrain, left hemisphere, right hemisphere regions of the nervous system. In some embodiments, the one or more regions of the nervous system comprises: right cortex, striatum, hippocampus, thalamus, cerebellum, or a combination thereof. In some embodiments, the one or more regions of the nervous system comprises: prefrontal cortex, sensory cortex, visual cortex, striatum, dorsal hippocampus, ventral hippocampus, thalamus, midbrain, cerebellum, or a combination thereof. In some embodiments, the one or more regions of the nervous systems comprises a dorsal root ganglia. In some embodiments, the nucleic acid complex is administered to the subject in need thereof via a subcutaneous injection, intravenous injection, intramuscular injection, intrastriatal injection, intrathecal injection, intracerebral injection, intracerebroventricular injection, intracranial injection, or a combination thereof. In some embodiments, the nucleic acid complex is administered to the subject in need thereof at a concentration about 0.001-10 nM, for example 0.004-1.0 nM.

In some embodiments, the nucleic acid complex is administered to the subject in need thereof at about 1-100 mg/kg body weight of the subject, optionally 10-50 mg/kg body weight of the subject. In some embodiments, the administration of the nucleic acid complex does not result in an increase or decrease in the body weight, inflammatory markers, blood chemistry, and/or liver, kidney pancreas enzymes in the subject. In some embodiments, the administration of the nucleic acid complex does not result in an unintended immunological response in the subject. In some embodiments, the target RNA is a mRNA or a miRNA. In some embodiments, the target RNA is a mRNA of a gene selected from HTT, APP, MAPT, SOD1, BACE1, CASP3, TGM2, NFE2L3, TARDBP, ADRB1, CAMK2A, CBLN1, CDK5R1, GABRA1, MAPK10, NOS1, NPTX2, NRGN, NTS, PDCD2, PDE4D, PENK, SYT1, TTR, FUS, LRDD, CYBA, ATF3, ATF6, CASP2, CASP1, CASP7, CASP8, CASP9, HRK, C1QBP, BNIP3, MAPK8, MAPK14, Rac1, GSK3B, P2RX7, TRPM2, PARG, CD38, STEAP4, BMP2, GJA1, TYROBP, CTGF, ANXA2, RHOA, DUOX1, RTP801, RTP801L, NOX4, NOX1, NOX2 (gp91pho, CYBB), NOX5, DUOX2, NOXO1, NOXO2 (p47phox, NCF1), NOXA1, NOXA2 (p67phox, NCF2), p53 (TP53), HTRA2, KEAP1, SHC1, ZNHIT1, LGALS3, HI95, SOX9, ASPP1, ASPP2, CTSD, CAPNS1, FAS and FASLG, NOGO and NOGO-R; TLR1, TLR2, TLR3, TLR4, TLR6, TLR7, TLR8, TLR9, IL1bR, MYD88, TICAM, TIRAP, HSP47, C3, GFAP, IBA-1, NPPA, CSF1R, SLC1A2, PLP1, MBP, ACCN3, SCN10A, Edg7, HTR3B, HTR3A, GPR64, NTRK1, CHRNA6, P2RX3, KCNK18, GAL, PRPH, CALCA, CALCB, P2RX3, and NAV1.7 gene.

In some embodiments, the neurological disease is Huntington's disease, Alzheimer's disease, Parkinson's disease, or Amyotrophic Lateral Sclerosis, neuropathic pain and the target RNA comprises the HTT, MSH3, SCNA, SOD1, GSK3B, MAPT, LRRK2, PUMA, ATXN2, CASP1, CD33, IKKB, NLRP3, RELA, RIPK1, or CD22, SCN9A, SCN10A, TRKA gene. In some embodiments, the third nucleic acid strand comprises, or consists of, the sequence of SEQ ID NO: 17 or 18, or a sequence comprising one or two mismatches of the sequence of SEQ ID NO: 17 or 18. In some embodiments, the second nucleic acid strand comprises, or consists of, the sequence of any one of SEQ ID NOs: 2, 19 and 20, or a sequence comprising one or two mismatches of any one of SEQ ID NOs: 2, 19 and 20; and/or the first nucleic acid strand comprises, or consists of, the sequence of SEQ ID NO: 1, or a sequence comprising one, two or three mismatches of SEQ ID NO: 1.

In some embodiments, the input nucleic acid strand comprises a cell-type and/or cell-state selective mRNA. In some embodiments, the input nucleic acid strand comprises one or more mRNAs of a gene selected from: C3, GFAP, IBA-1, NPPA, CSF1R, SLC1A2, PLP1, MBP, ACCN3, SCN10A, Edg7, HTR3B, HTR3A, GPR64, NTRK1, CHRNA6, P2RX3, KCNK18, GAL, PRPH, CALCA, CALCB, P2RX3, NAV1.7 and a combination thereof.

The input nucleic acid strand can comprise a universal mRNA that is not cell-type or cell-state selective. In some embodiments, the input nucleic acid strand comprises one or more miRNAs selected from the group consisting of mir-21-5p, mir-23a-3p, mir-29c-3p, mir-29b-3p, mir-124-3p, and 5.8s ribosomal RNA. In some embodiments, the nucleic acid complex is administered to a subject in the form of a pharmaceutical composition. In some embodiments, the nucleic acid complex is administered to a subject via a lipid-mediated delivery system, optionally via liposomes, nanoparticles, or micelles. In some embodiments, the nucleic acid complex is administered to a subject via nanoparticles, inorganic nanoparticles, nucleic acid lipid particles, polymeric nanoparticles, lipidoid nanoparticles, lipid nanoparticles (LNPs), chitosan and inulin nanoparticles, cyclodextrins nanoparticles, carbon nanotubes, liposomes, micellar structures, capsids, polymers, polymer matrices, hydrogels, dendrimers, nucleic acid nanostructure, exosomes, GalNAc-conjugated melittin-like peptides, or combinations thereof.

In some embodiments, the administration of the nucleic acid complex results in a reduction of at least 50% or at least 75% with respect to the level of the target RNA prior to the administration.

In some embodiments, the administration of the nucleic acid complex results in a reduction of at least 50% or at least 75% with respect to the level of protein expression from the target RNA prior to the administration. In some embodiments, the reduction in the target RNA level and/or the reduction in the protein expression level from the target RNA lasts for at least 30 days, at least 60 days, at least 90 days, or at least 120 days after the administration, and optionally the reduction is the reduction in anterior spinal cord, posterior spinal cord, left hemisphere, right hemisphere, or any combination thereof. In some embodiments, the reduction in the target RNA level and/or the reduction in the protein expression level from the target RNA is determined at or after 30 days, 60 days, 90 days, or 120 days after administration, and optionally the reduction is the reduction in anterior spinal cord, posterior spinal cord, left hemisphere, right hemisphere, or any combination thereof.

In some embodiments, the method comprises determining the target RNA level and/or the protein expression level from the target RNA prior to the administration, after the administration, or both. In some embodiments, the reduction occurs in one or more of the regions comprising right cortex, prefrontal cortex, sensory cortex, visual cortex, striatum, dorsal hippocampus, ventral hippocampus, thalamus, cerebellum, midbrain, left hemisphere, right hemisphere, or a combination thereof. In some embodiments, the administration is performed two or more times and wherein two administrations of the nucleic acid complex are separated by at least 6 months.

### BRIEF DESCRIPTION OF THE DRAWINGS

FIG. 1 illustrates a schematic representation of two non-limiting exemplary nucleic acid complex constructs.

FIG. 2 illustrates a schematic representation of a non-limiting exemplary nucleic acid complex construct.

FIG. 3 illustrates a schematic representation of two non-limiting exemplary nucleic acid complex constructs.

FIG. 4 illustrates a schematic representation of a sensor nucleic acid strand, a core nucleic acid strand and a passenger nucleic acid strand of a non-limiting exemplary nucleic acid complex.

FIG. 5 illustrates a schematic representation of a non-limiting exemplary nucleic acid complex construct with regions for screening highlighted in yellow.

FIG. 6 is a schematic diagram showing the formation of an active RNAi duplex following the displacement of a sensor nucleic acid strand from a core nucleic acid strand and the degradation of the core nucleic acid strand overhangs.

FIG. 7 demonstrates the stability of CASi complexes to serum degradation over time.

FIG. 8 demonstrates the localization of siRNA in cells.

FIG. 9A is a microscopic image showing the distribution of CASi in the central nervous system (CNS) 48 hours after a CASi injection. FIG. 9B is a microscopic image showing the CNS cellular uptake of CASi 48 hours after the injection.

FIG. 10A is a diagram illustrating the body weights of the group administered with CASi C in comparison to a control group administrated with saline. FIG. 10B provides diagrams showing aspartate aminotransferase (AST) (U/L), alanine aminotransferase (ALT) (U/L), IL-6 (pg/mL), matrix metalloproteinase-1 (MMP-1) (ng/ml), vascular cell adhesion molecule 1 (VCAM-1) (ng/mL), IL-8 (pg/mL), monocyte chemoattractant protein-1 (MCP-1) (pg/mL), C-reactive protein (CRP) (μg/mL) of the group administered with CASi C in comparison to a control group.

FIG. 11A-B depicts graphs showing the glial fibrillary acidic protein mRNA (GFAP mRNA) (FIG. 11A) and ionized calcium-binding adapter molecule 1 mRNA (IBA-1 mRNA) (FIG. 11B) levels in various brain regions of CASi treated animals (t) in comparison to saline treated animals (c).

FIG. 12 depicts graphs showing the RNAi activity of exemplary siRNA candidates.

FIG. 13A illustrates a schematic representation of a non-limiting exemplary nucleic acid complex construct T2 CASi. FIG. 13B shows sequence diagrams of CASi A construct comprising a sensor strand (SEQ ID NO: 18), a core strand (SEQ ID NO: 19 and SEQ ID NO: 20 joined together via a C3 spacer) and a passenger strand (SEQ ID NO: 1). /5Sp9/=5′ triethylene glycol spacer; /3AmMC6T/=3′ amino modifier C6 dT.

FIG. 14A illustrates a schematic representation of a non-limiting exemplary nucleic acid complex construct T1 CASi. FIG. 14B shows sequence diagrams of CASi B construct comprising a sensor strand (SEQ ID NO: 17), a core strand (SEQ ID NO: 2) and a passenger strand (SEQ ID NO: 1). FIG. 14C-E illustrates the chemical formulas of an exemplary sensor strand (FIG. 14C: SEQ ID NO: 17), passenger strand (FIG. 14D: SEQ ID NO: 1) and core strand (FIG. 14E: SEQ ID NO: 2), respectively. FIG. 14F shows an exemplary formulation of making a T1 CASi construct. FIG. 14G shows the results from polyacrylamide gel electrophoresis (PAGE) analysis of T1 CASi constructs, individual strands and duplexes.

FIG. 15A shows a graphic representation of the target protein expression data for Sensor 1 CASi construct and the siRNA domain of the Sensor 1 CASi construct. FIG. 15B shows a graphic representation of the target protein expression data for CASi A construct in cells expressing a cell type specific mRNA (e.g., GFAP mRNA) (“+mRNA A”) and cells not expressing the cell type specific mRNA (“-mRNA A”). FIG. 15C shows a graphic representation of the target protein expression data for CASi B construct, the siRNA domain of the CASi B construct, Sensor 1 CASi and the siRNA domain of the Sensor 1 CASi.

FIG. 16 are graphs showing the target protein expression data of the universal CASi constructs listed in Table 1.

FIG. 17 is a graph showing the target protein expression data in various brain regions of CASi B treated animals (t) in comparison to saline treated animals (c).

FIG. 18 depicts graphs showing the target protein expression data in various brain regions 14 days, 30 days and 90 days after CASi administration.

FIG. 19 depicts graphs showing the target mRNA levels in the spinal cord of the mice treated with the CASi constructs in comparison to saline treated mice 30 days and 90 days after the administration.

FIG. 20 depicts a graph showing the target mRNA levels in various regions of the nervous system 30 days after CASi administration.

FIG. 21 depicts graphs showing the target mRNA levels in various brain regions of the mice treated with the T1 CASi construct without a 3′ terminal palmitic acid (top panel) and with a 3′ terminal palmitic acid (bottom panel) 14 days after CASi injection.

FIG. 22 depicts a diagram showing the target mRNA levels in various brain regions of the mice treated with a CASi construct having a standard 8 nucleotide toehold (with or without palmitic acid) (8 nt toe+PA or 8 nt toe), with a CASi construct having a 12 nucleotide toehold (12 nt toe), or with a CASi construct having a 16 nucleotide toehold (16 nt toe).

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

RNA interference (RNAi) is an intrinsic cellular mechanism conserved in most eukaryotes, that helps to regulate the expression of genes critical to cell fate determination, differentiation, survival and defense from viral infection. Researchers have exploited this natural mechanism by designing synthetic double-stranded RNA for sequence-specific gene silencing. Emerging developments in the field of dynamic nucleic acid nanotechnology and biomolecular computing also offer a conceptual approach to design programmable RNAi agents. However, challenges still remain in developing targeted RNAi therapy that can use nucleic acid logic switches to sense RNA transcripts (such as mRNAs and miRNAs) in order to restrict RNA silencing to specific populations of disease-related cells and spare normal tissues from toxic side effects. Significant challenges include poorly suppressed background drug activity, weak activated state drug potency, input and output sequence overlap, high design complexity, short lifetimes (<24 hours) and high required device concentrations (>10 nM).

RNAi agents can be used to silence genes involved in the pathogenesis of various diseases associated with a known genetic background. As for many neurodegenerative disorders a causative therapy is unavailable, siRNA holds a promising option for the development of novel therapeutic strategies.

Provided herein include methods, compositions, and kits suitable for use in preventing and treating a neurological disease or disorder (e.g., a disease or disorder of the central nervous system). The method can comprise administering to a subject in need thereof a composition comprising conditionally activatable small interfering RNA (CASI RNA or siRNA) complexes. The CASi RNA complex or siRNA complex herein described can switch from an inactivated state to an activated state when triggered by a complementary binding of an input nucleic acid strand (e.g., a neurological disease biomarker gene) to the siRNA complex, thereby activating the RNA interference activity of the siRNA complex to target a specific target RNA (e.g., a RNA to be silenced). In some embodiments, the methods can mediate conditionally activated RNA interference activity to silence a specific disease gene in disease-related cells with improved potency at a low concentration, improved specificity that reduces off-target effects while maintaining safety and tolerability in the subject being treated. In some embodiments, the methods herein described can reduce the target nucleic acid associated with a neurological disease or disorder (e.g., Huntington's disease) in one or more regions of the nervous system (e.g., cerebral cortex, striatum, hippocampus, thalamus, cerebellum) by at least 50%.

### Definitions

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “about” means plus or minus 5% of the provided value.

As used herein, the term “nucleoside” refers to a molecule having a purine or pyrimidine base covalently linked to a ribose or deoxyribose sugar. Exemplary nucleosides include adenosine, guanosine, cytidine, uridine and thymidine.

The term “nucleotide” refers to a nucleoside having one or more phosphate groups joined in ester linkages to the sugar moiety. Exemplary nucleotides include nucleoside monophosphates, diphosphates and triphosphates.

The terms “polynucleotide” and “nucleic acid molecule” are used interchangeably herein and refer to a polymer of nucleotides joined together by a phosphodiester linkage between 5′ and 3′ carbon atoms.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded or multi-stranded (e.g., double-stranded or triple-stranded). “mRNA” or “messenger RNA” is single-stranded RNA molecule that is complementary to one of the DNA strands of a gene. “miRNA” or “microRNA” is a small single-stranded non-coding RNA molecule that functions in RNA silencing and post-transcriptional regulation of gene expression.

The term “RNA analog” refers to an polynucleotide having at least one altered or modified nucleotide as compared to a corresponding unaltered or unmodified RNA. The nucleotide can retain the same or similar nature or function as the corresponding unaltered or unmodified RNA such as forming base pairs.

A single-stranded polynucleotide has a 5′ terminus or 5′ end and a 3′ terminus or 3′ end The terms “5′ end” “5′ terminus” and “3′ end” “3′ terminus” of a single-stranded polynucleotide indicate the terminal residues of the single-stranded polynucleotide and are distinguished based on the nature of the free group on each extremity. The 5′-terminus of a single-stranded polynucleotide designates the terminal residue of the single-stranded polynucleotide that has the fifth carbon in the sugar-ring of the deoxyribose or ribose at its terminus (5′ terminus). The 3′-terminus of a single-stranded polynucleotide designates the residue terminating at the hydroxyl group of the third carbon in the sugar-ring of the nucleotide or nucleoside at its terminus (3′ terminus). The 5′ terminus and 3′ terminus in various cases can be modified chemically or biologically e.g., by the addition of functional groups or other compounds as will be understood by the skilled person.

As used herein, the terms “complementary binding” and “bind complementarily” mean that two single strands are base paired to each other to form nucleic acid duplex or double-stranded nucleic acid. The term “base pair” as used herein indicates formation of hydrogen bonds between base pairs on opposite complementary polynucleotide strands or sequences following the Watson-Crick base pairing rule. For example, in the canonical Watson-Crick DNA base pairing, adenine (A) forms a base pair with thymine (T) and guanine (G) forms a base pair with cytosine (C). In RNA base paring, adenine (A) forms a base pair with uracil (U) and guanine (G) forms a base pair with cytosine (C). A certain percentage of mismatches between the two single strands are allowed as long as a stable double-stranded duplex can be formed. In some embodiments, the two strands that bind complementarily can have a mismatches can be, about, be at most, or be at most bout 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%.

As used herein, the terms “RNA interference”, “RNA interfering”, and “RNAi” refer to a selective intracellular degradation of RNA. RNAi can occur in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

As used herein, the terms “small interfering RNA” and “siRNA” refer to an RNA or RNA analog capable of reducing or inhibiting expression of a gene or a target gene when the siRNA is activated in the same cell as the target gene. The siRNA used herein can comprise naturally occurring nucleic acid bases and/or chemically modified nucleic acid bases (RNA analogs).

As used herein, “treatment” refers to a clinical intervention made in response to a disease, disorder or physiological condition manifested by a patient or to which a patient may be susceptible. The aim of treatment includes, but is not limited to, the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Subjects in need of treatment include those already affected by a disease or disorder or undesired physiological condition as well as those in which the disease or disorder or undesired physiological condition is to be prevented.

As used herein, the term “prophylaxis,” “prevent,” “preventing,” “prevention,” and grammatical variations thereof as used herein refers the preventive treatment of a subclinical disease-state in a subject, e.g., a mammal (including a human), for reducing the probability of the occurrence of a clinical disease-state. The method can partially or completely delay or preclude the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms. The subject is selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, the term “pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjuster controller, isotonic agent and other conventional additives may also be added to the carriers.

As used herein, the terms “effective amount” or “pharmaceutically effective amount” or “therapeutically effective amount” refer to an amount sufficient to effect beneficial or desirable biological and/or clinical results.

The term “pharmaceutically acceptable excipient” as used herein refers to any suitable substance that provides a pharmaceutically acceptable carrier, additive or diluent for administration of a compound(s) of interest to a subject. Pharmaceutically acceptable excipient can encompass substances referred to as pharmaceutically acceptable diluents, pharmaceutically acceptable additives, and pharmaceutically acceptable carriers.

As used herein, a “subject” refers to an animal for whom a diagnosis, treatment, or therapy is desired. In some embodiments, the subject is a mammal. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a primate. In some embodiments, the mammal is a human. In some embodiments, the mammal is not a human.

### Nucleic Acid Complexes

Provided herein include a nucleic acid complex that can be conditionally activated upon a complementary binding to an input nucleic acid strand (e.g., a mRNA of a disease biomarker gene specific to a target cell (e.g., disease-related cells)) through a sequence in a sensor nucleic acid strand of the nucleic acid complex. The activated nucleic acid complex can release a potent RNAi duplex formed by a core nucleic acid strand and a passenger nucleic acid strand, which can specifically inhibit or silence a target RNA. The target RNA can have a sequence independent from the input nucleic acid strand. The target RNA can be from a gene that is different from the gene that the input nucleic acid strand is from. In some embodiments, the target RNA is from a gene that is the same as the gene that the input nucleic acid strand is from. The nucleic acid complexes and related compositions and kits can be used to prevent or treat a neurological disease or disorder, e.g., a disease or disorder of the central nervous system.

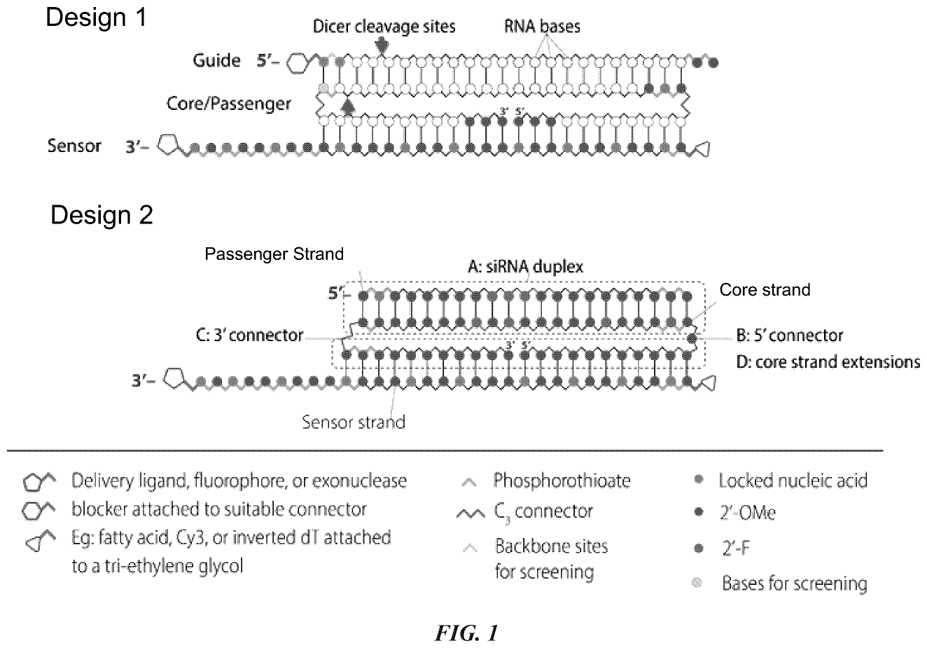

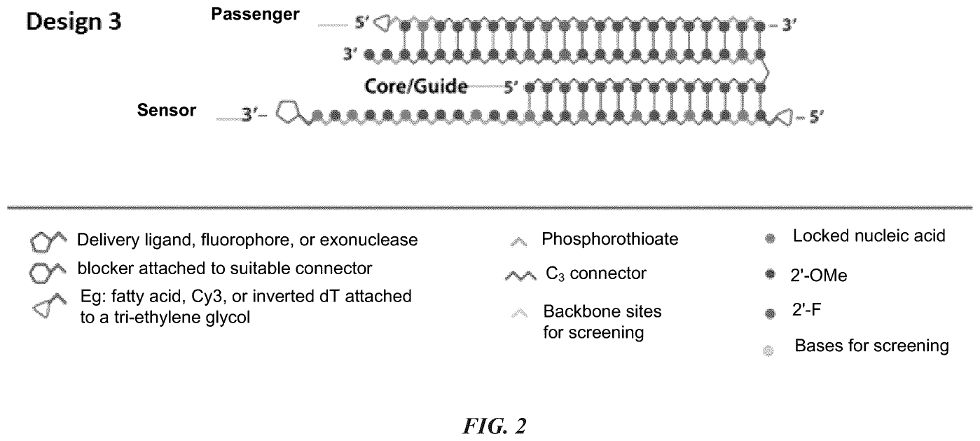

FIGS. 1-3 illustrates schematic representations of non-limiting exemplary nucleic acid complex constructs.

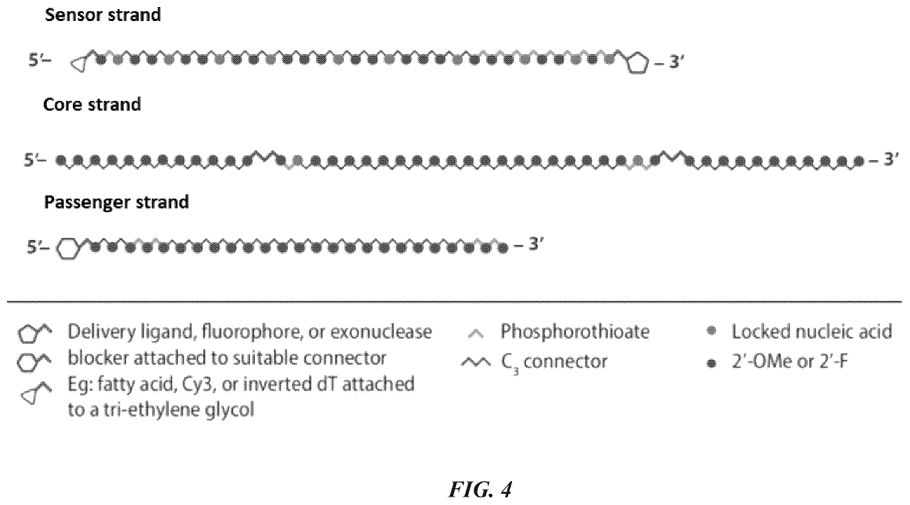

In some embodiments, the nucleic acid complexes described herein comprise a core nucleic acid strand, a passenger nucleic acid strand, and a sensor nucleic acid strand as shown in a non-limiting embodiment of FIG. 4. These three strands can base-pair with one another to form, for example, a RNAi duplex and a sensor duplex. One or more of the core nucleic acid strand, the passenger nucleic acid strand, and the sensor nucleic acid strand can be RNA analogs comprising modified nucleotides.

The term “nucleic acid duplex” as used herein refers to two single-stranded polynucleotides bound to each other through complementarily binding. The nucleic acid duplex can form a helical structure, such as a double-stranded RNA molecule, which is maintained largely by non-covalent bonding of base pairs between the two single-stranded polynucleotides and by base stacking interactions.

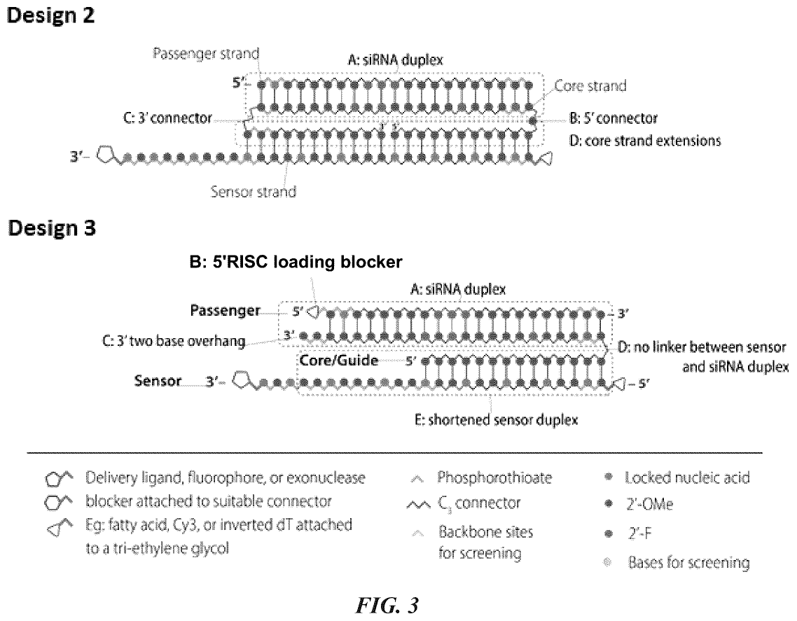

In some embodiments, the core nucleic acid strand of a nucleic acid complex herein described can comprise a 5′ region, a 3′ region, and a central region between the 5′ region and the 3′ region (see e.g., in FIG. 1 and FIG. 3). The central region of the core nucleic acid strand can be linked to the 5′ region and/or the 3′ region of the core nucleic acid strand via a connector. In some embodiments, the central region of the core nucleic acid strand is linked the 5′ region of the core nucleic acid strand via a 5′ connector. In some embodiments, the central region of the core nucleic acid strand is linked to the 3′ region of the core nucleic acid strand via a 3′ connector. The central region of the core nucleic acids strand is complementarily bound to the passenger nucleic acid strand to form a RNAi duplex. Not the entire sequence of the core nucleic acid strand is complementarily bound to the passenger nucleic acid strand. For example, the 5′ region and the 3′ region of the core nucleic acid strand is not complementarily bound to the passenger nucleic acid strand.

The core nucleic acid strand can comprise a first region and a second region and the first region is at the 3′ direction of the second region (see e.g., in FIGS. 2-3). In other words, the first region is at the 3′ end of the core nucleic acid strand and the second region is at the 5′ end of the core nucleic acid strand. The first region of the core nucleic acid strand can be linked to the second region of the core nucleic acid strand via a connector, which can also be referred to as a 5′ connector. The 5′ connector can be a normal phosphodiester internucleoside linkage connecting two adjacent nucleotides. In some embodiments, the core nucleic acid strand only comprises one connector (e.g., 5′ connector) and does not comprise a 3′ connector. The first region of the core nucleic acids strand is complementarily bound to the passenger nucleic acid strand to form a RNAi duplex. Not the entire sequence of the core nucleic acid strand is complementarily bound to the passenger nucleic acid strand. For example, the second region of the core nucleic acid strand is not complementarily bound to the passenger nucleic acid strand. In some embodiments, the first region of the core nucleic acid strand is fully complementary to the passenger nucleic acid strand, thereby forming a RNAi duplex having a blunt end with no overhang at the 5′ and 3′ termini of the first region of the core nucleic acid strand. In some embodiments, the core nucleic acid strand of the RNAi duplex has a short overhang at the 3′ terminus (e.g., one, two, or three nucleosides), but the 3′ overhang does not extend back into the middle of the sensor duplex to bind with the sensor nucleic acid strand (see e.g., in FIGS. 2-3). In some embodiments, the core nucleic acid strand does not have any region at the 3′ of the first region of the core nucleic acid strand.

The core nucleic acid strand (e.g., the central region in Design 1 and Design 2 or the first region in Design 3) can comprise a sequence complementary to a target nucleic acid (e.g., a RNA to be silenced). The core nucleic acid strand of the nucleic acid complex therefore acts as a guide strand (antisense strand) and is used to base pair with a target RNA. The passenger nucleic acid strand can therefore comprise a sequence homologous to the same target nucleic acid.

Upon activation of the nucleic acid complex (e.g., binding to an input nucleic acid strand), the released RNAi duplex can complementarily bind a target nucleic acid through the binding between the target nucleic acid and the core nucleic acid strand. In some embodiments, the sequence complementary to a target RNA in the core nucleic acid strand can be about 10-35 nucleosides in length. In some embodiments, the core nucleic acid strand comprises 20-70 linked nucleosides. In some embodiments, the core nucleic acid strand comprises 20-60 linked nucleosides.

In some embodiments wherein the core nucleic acid strand comprises a 5′ region, a central region and a 3′ region (e.g., in FIG. 1 and FIG. 2), the sensor nucleic acid strand is complementarily bound to the 5′ region and the 3′ region of the core nucleic acid strand to form a sensor duplex. The sensor nucleic acid strand does not bind to the central region of the core nucleic acid strand nor the passenger nucleic acid strand.

In some embodiments wherein the core nucleic acid strand comprises a first region and a second region (e.g., in FIGS. 2-3), the sensor nucleic acid strand is complementarily bound to the second region of the core nucleic acid strand to form a sensor duplex. The sensor nucleic acid strand does not bind to the first region of the core nucleic acid strand nor any region of the core nucleic acid strand that is 3′ of the first region of the core nucleic acid strand. The sensor nucleic acid strand also does not bind to the passenger nucleic acid strand.

The sensor nucleic acid strand can comprise an overhang. The term “overhang” as used herein refers to a stretch of unpaired nucleotides that protrudes at one of the ends of a double-stranded polynucleotide (e.g., a duplex). An overhang can be on either strand of the polynucleotide and can be included at either the 3′ terminus of the strand (3′ overhang) or at the 5′ terminus of the strand (5′ overhang). The overhang can be at the 3′ terminus of the sensor nucleic acid strand. The overhang of the sensor nucleic acid strand does not bind to any region of the core nucleic acid strand.

The sensor nucleic acid strand can comprise a sequence capable of binding to an input nucleic acid strand (e.g., a mRNA of a disease biomarker gene specific to a target cell, including a disease-related cell). Upon activation, the binding of the sensor nucleic acid strand to the input nucleic acid strand can cause displacement and subsequent release of the sensor nucleic acid strand from the core nucleic acid strand, thereby releasing the potent RNAi duplex and switching on the RNA interfering activity of the RNAi duplex. In the absence of an input nucleic acid strand or a detectable amount of the input nucleic acid strand, the nucleic acid complex herein described remains in an inactivated state (switched off) and the displacement of the sensor nucleic acid strand from the core nucleic acid strand does not take place. Therefore, the input nucleic acid strand can act as a trigger to activate (switch on) the RNA interfering activity of the nucleic acid complex (e.g., RNAi duplex).

The length of the RNAi duplex of the nucleic acid complex herein described can vary in different embodiments. In some embodiments, the length of the RNAi duplex can be 10-35 nucleotides, optionally 10-30 nucleotides. For example, the length of the RNAi duplex can be, 10, 11, 12, 13, 14, 15, 16, 17, 18, 19, 20, 21, 22, 23, 24, 25, 26, 27, 28, 29, 30, 31, 32, 33, 34, 35, a range of any two of these values, nucleotides. In some embodiments, the length of the RNAi duplex can be 19-25 nucleotides. In some embodiments, the length of the RNAi duplex can be 17-22 nucleotides.

The length of the sensor duplex of the nucleic acid complex herein described can vary in different embodiments. In some embodiments, the length of the sensor duplex can be 10-35 nucleotides, optionally 10-30 nucleotides. For example, the length of the sensor duplex can be, 10, 11, 12, 13, 14, 15, 16, 17, 18, 19, 20, 21, 22, 23, 24, 25, 26, 27, 28, 29, 30, 31, 32, 33, 34, 35, a range of any two of these values, nucleotides. In some embodiments, the length of the sensor duplex is about 14 nucleotides. In some embodiments, the sensor duplex has a relatively short length with respect to the RNAi duplex.

In some embodiments, there is no linker molecule between a sensor duplex and a RNAi duplex of a nucleic acid complex except for the normal phosphodiester linkage connecting two adjacent nucleosides each located at a terminus of one of the two duplexes.

In some embodiments, the sensor duplex formed by a portion of a core nucleic acid strand and a portion of a sensor nucleic acid strand can comprise one or more wobble base pair or mismatch. The term “wobble base pair” as used herein refers to a base pairing between two nucleotides in a nucleic acid duplex that does not follow Watson-Crick base pair rules. Wobble base pairs can include guanine-uracil (G-U), hypoxanthine-uracil (I-U), hypoxanthine-adenine (I-A), and hypoxanthine-cytosine (I-C). The denotation “I” refers to hypoxanthine which forms an inosine when attached to a ribose ring. Details on the wobble base pairings and their possible locations in the sensor duplex can be found in U.S. Provisional Application No. 63/218,850 filed on Jul. 6, 2021, which is incorporated herein by reference. In some embodiments, the bond strength or base pair strength in a portion of the sensor duplex is reduced due to the presence of the wobble base pairs (e.g., G-U, I-U, I-A, and/or I-C). Therefore, in some embodiments, the wobble base pairs in the sensor duplex can decrease the thermodynamic stability of the sensor duplex, such as to lower the melting temperature of the sensor duplex, thereby promoting the toehold-mediated displacement of the sensor nucleic acid strand from the core nucleic acid strand triggered by an input nucleic acid strand.

The nucleic acid complexes herein described can be synthesized using standard methods for oligonucleotide synthesis well-known in the art including, for example, Oligonucleotide Synthesis by Herdewijin, Piet (2005) and Modified oligonucleotides: Synthesis and Strategy for Users, by Verma and Eckstein, Annul Rev. Biochem. (1998): 67:99-134, the contents of which are incorporated herein by reference in their entirety. The synthesized nucleic acid complexes can be allowed to form its secondary structure under a desirable physiological condition as will be apparent to a skilled artisan. The formed secondary structure can be tested using standard methods known in the art such as chemical mapping, NMR, or computational simulations. The nucleic acid complex construct can be further modified, according to the test result, by introducing or removing chemical modifications or mismatches, as necessary, until the desired structure is obtained.

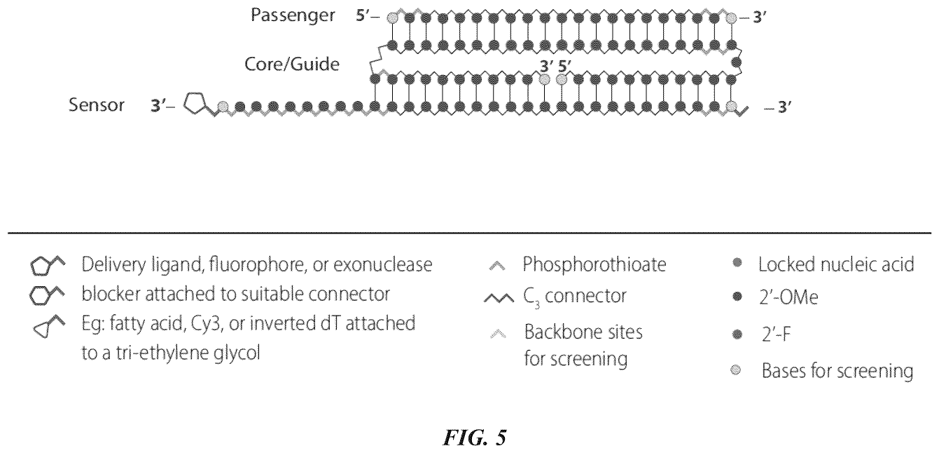

Suitable software suites can be used to aid in the design and analysis of nucleic acid structures. For example, Nupack can be used to check the formation of the duplexes and to rank the thermodynamic stability of the duplexes. Oligonucleotide design tools can be used to optimize the placement of LNA modifications. Any of the regions of one or more of the strands in a nucleic acid complex herein described can be screened for an input nucleic acid sequence, a target nucleic acid sequence and/or chemical modifications herein described. For example, FIG. 5 illustrates a schematic representation of a non-limiting exemplary nucleic acid complex construct, highlighting in yellow the terminal bases that can be screened for chemical modifications such as LNA placements and other nucleotide analogs herein described.

### RNA Interference (RNAi)

Described herein include nucleic acid complexes that can be conditionally activated (e.g., via a signal for the presence of a mRNA of a gene specific for a target cell) to switch from an assembled, inactivated state to an activated state to act on (e.g., degrade or inhibit) a specific target nucleic acid in response to the detection of an input nucleic acid (e.g., a nucleic acid sequence specific to a target cell, including a disease-related cell) having a sequence complementary to a sequence in the sensor nucleic acid strand of a nucleic acid complex.

In the assembled, inactivated configuration, the sensor nucleic acid strand of the nucleic acid complex inhibits enzymatic processing of the RNAi duplex, thereby keeping RNAi activity switched off.

In the event that an input nucleic acid strand complementary to the sensor nucleic acid strand of a nucleic acid complex is present, the input nucleic acid strand can activate the nucleic acid complex by inducing separation of the sensor nucleic acid strand from the core nucleic acid strand via toehold mediated strand displacement. Displacement can start from a toehold formed at the 3′ or 5′ terminus of the sensor nucleic acid strand (e.g., a 5′ toehold or a 3′ toehold) through a complementary binding between the input nucleic acid strand and an overhang of the sensor nucleic acid strand.

After removal of the sensor nucleic acid strand, the region of the core nucleic acid strand that is not complementary bound to the passenger nucleic acid strand become an overhang region that can be degraded by nucleases (e.g., exonuclease). For example, in some embodiments (e.g., in FIG. 1), the 3′ and 5′ region of the core nucleic acid strand become 3′ and 5′ overhangs. In some other embodiments (e.g., in FIGS. 2-3), the second region of the core nucleic acid strand becomes a 5′ overhang.

This degradation stops at the 3′ end and/or 5′ end of the RNAi duplex due to the presence of chemically modified nucleotides and/or exonuclease cleavage-resistance moieties, thus rendering an active RNAi duplex for further endonuclease processing if needed and RNA-induced silencing complex (RISC) loading.

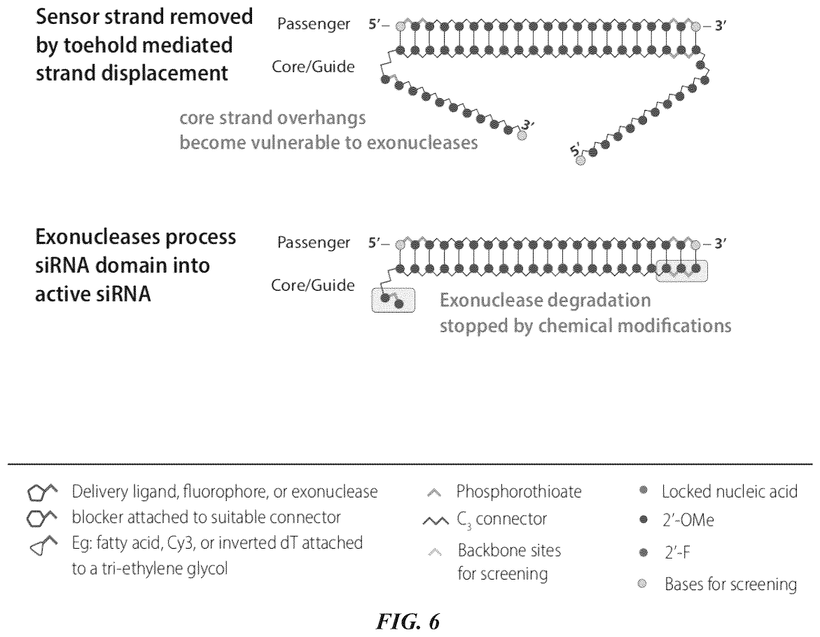

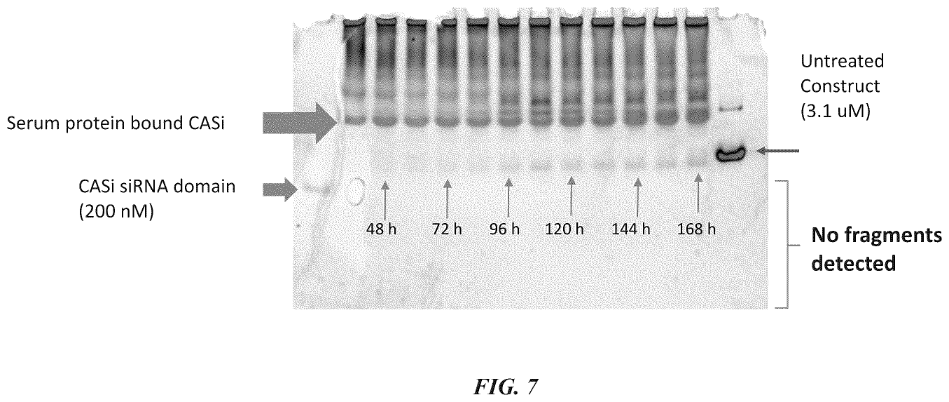

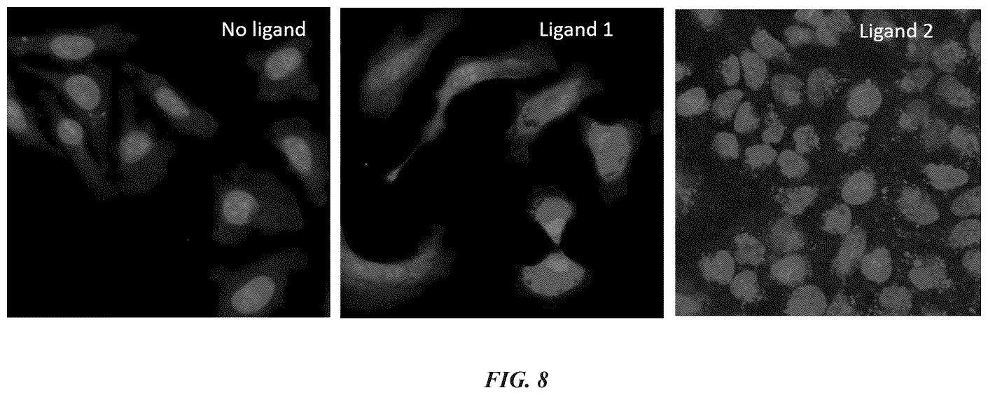

FIG. 6 is a non-limiting schematic diagram showing the formation of an active RNAi duplex following the displacement of a sensor nucleic acid strand from a core nucleic acid strand and the degradation of the core nucleic acid strand overhangs.

RISC is a multiprotein complex that incorporates one strand of a siRNA or miRNA and uses the siRNA or miRNA as a template for recognizing complementary target nucleic acid. Once a target nucleic acid is identified, RISC activates RNase (e.g., Argonaute) and inhibits the target nucleic acid by cleavage. In some embodiments, Dicer is not required for loading the RNAi duplex into RISC.

The passenger nucleic acid strand is then discarded, while the core nucleic acid strand is incorporated into RICS. The core nucleic acid strand of the nucleic acid complex disclosed herein acts as a guide strand (antisense strand) and is used to base pair with a target RNA. The passenger nucleic acid strand acts as a protecting strand prior to the loading of the core nucleic acid strand into RICS. RICS uses the incorporated core nucleic acid strand as a template for recognizing a target RNA that has complementary sequence to the core nucleic acid strand, particularly the central region of the core nucleic acid strand. Upon binding to the target RNA, the catalytic component of RICS, Argonaute, is activated which can degrade the bound target RNA. The target RNA can be degraded or the translation of the target RNA can be inhibited.

In some embodiments, the nucleic acid complexes generated herein do not have a dicer cleavage site, and therefore the RNAi interference mediated by the nucleic acid complexes can bypass Dicer-mediated cleavage. Dicer is an endoribonuclease in the RNAse III family that can initiate the RNAi pathway by cleaving double-stranded RNA (dsRNA) molecule into short fragments of dsRNAs about 20-25 nucleotides in length. In some embodiments, the nucleic acid complexes generated herein differentiate from the conditionally activated small interfering RNAs (Cond-siRNAs) disclosed in WO2020/033938 in that the nucleic acid complexes generated herein can bypass the Dicer processing.

In some embodiments, the nucleic acid complexes generated herein have structural features that discourage the Dicer binding. In some embodiments, the RNAi duplex does not create a Dicer substrate. For example, the RNAi duplex formed by the passenger nucleic acid strand and the core nucleic acid strand do not have a 3′ and/or 5′ overhang, but instead forming a blunt end that can render the passenger nucleic acid strand unfavorable for Dicer binding. In some embodiments, the passenger nucleic acid strand has about 17-22 nucleotides in length, making it short enough to bypass Dicer cleavage. In some embodiments, the passenger nucleic acid strand does not have G/C rich bases to the 3′ and/or 5′ end of the passenger nucleic acid strand. In some embodiments, the passenger nucleic acid strand are attached to a terminal moiety to avoid Dicer binding.

Upon activation, the nucleic acid complex generated herein can inhibit a target nucleic acid in target cells, therefore resulting in a reduction or loss of expression of the target nucleic acid in the target cells. The target cells are cells associated or related to a disease or disorder. The term “associated to” “related to” as used herein refers to a relation between the cells and the disease or condition such that the occurrence of a disease or condition is accompanied by the occurrence of the target cells, which includes but is not limited to a cause-effect relation and sign/symptoms-disease relation. The target cells used herein typically have a detectable expression of an input nucleic acid.

In some embodiments, the expression of a target nucleic acid in target cells is inhibited about, at least, at least about, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any of these values.

As used herein, inhibition of gene expression refers to the absence or observable decrease in the level of protein and/or mRNA product from a target gene in target cells. The degree of inhibition can be evaluated by examination of the expression level of the target gene as demonstrated in the examples.

Gene expression and/or the inhibition of target gene expression can be determined by use of a reporter or drug resistance gene whose protein product is easily assayed. Exemplary reporter genes include, but no limited to, acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentarnycin, hygromycin, kanamycin, lincomycin, methotrexate, phosphinothricin, puromycin, and tetracyclin. Quantitation of the amount of gene expression allows one to determine a degree of inhibition as compared to cells not treated with the nucleic acid complexes or treated with a negative or positive control. Various biochemical techniques may be employed as will be apparent to a skilled artisan such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

In some embodiments, the nucleic acid complexes generated herein exhibit improved switching performance and reduced off-target effects. The nucleic acid complexes generated herein can have a reduced unwanted RNAi activity when the nucleic acid complexes are in an inactivated state (switched off) and an enhanced RNAi activity when the nucleic acid complexes are activated upon detection of an input nucleic acid strand.

In some embodiments, the expression of a target nucleic acid in non-target cells (e.g., cells not having an input nucleic acid strand) is inhibited about, at most, or at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any of these values. Non-target cells can comprise cells of the subject other than target cells.

In some embodiments, the nucleic acid complexes generated herein have an enhanced potency, thus capable of evoking an RNAi activity at low concentrations. Nonspecific, off-target effects and toxicity (e.g., undesired proinflammatory responses) can be minimized by using low concentrations of the nucleic acid complexes.

The concentration of the nucleic acid complexes generated herein can vary in different embodiments. In some embodiments, the nucleic acid complexes generated herein can be provided at a concentration of, about, at most, or at most about, 0.001 nM, 0.01 nM, 0.02 nM, 0.03 nM, 0.04 nM, 0.05 nM, 0.06 nM, 0.07 nM, 0.08 nM, 0.09 nM, 0.1 nM, 0.2 nM, 0.3 nM, 0.4 nM, 0.5 nM, 0.6 nM, 0.7 nM, 0.8 nM, 0.9 nM, 1.0 nM, 1.5 nM, 2.0 nM, 2.5 nM, 3.0 nM, 3.5 nM, 4.0 nM, 4.5 nM, 5.0 nM, 5.5 nM, 6.0 nM, 6.5 nM, 7.0 nM, 7.5 nM, 8.0 nM, 8.5 nM, 9.0 nM, 9.5 nM, 10 nM, 11 nM, 12 nM, 13 nM, 14 nM, 15 nM, 16 nM, 17 nM, 18 nM, 19 nM, 20 nM, 30 nM, 40 nM, 50 nM, or a number or a range between any two of these values. For example, the nucleic acid complexes generated herein can be provided at a concentration between about 0.1-10 nM, preferably between about 0.1-1.0 nM. In some embodiments, the nucleic acid complex generated herein has a transfection concentration at about 0.1 nM or lower.

The nucleic acid complex herein described can allow lasting and consistently potent inhibition effects at low concentrations. For example, the nucleic acid complex can remain active for an extended period of time such as 1 hour, 2 hours, 6 hours, 12 hours, 24 hours, 36 hours, 48 hours, 60 hours, 72 hours, 84 hours, 96 hours, 5 days, 6 days, 7 days, two weeks, or a number or a range between any of these values, or more. In some embodiments, the nucleic acid complex can remain active for at least 12 hours, at least 24 hours, at least 36 hours, at least 48 hours, at least 60 hours, at least 72 hours, at least 84 hours, or at least 96 hours. In some embodiments, the nucleic acid complex can remain active for up to 30 days, up to 60 days, or up to 90 days.

### Core Strand

The core nucleic acid strand of the nucleic acid complex described herein can comprise a 5′ region, a 3′ region, and a central region between the 5′ region and the 3′ region. Each of the 5′ region, the 3′ region, and the central region can be directly adjacent to each other, that is no nucleotide between the two adjacent regions. In some embodiments, the 3′ end of the 5′ region can be 1, 2, 3, 4, 5, 8, 9, 10, 11, 12, 13, 14, 15, 20, or a number or a range between any two of these values, nucleotides away from the 5′ end of the central region. In some embodiments, the 5′ end of the 3′ region can be 1, 2, 3, 4, 5, 8, 9, 10, 11, 12, 13, 14, 15, 20, or a number or a range between any two of these values, nucleotides away from 3′ of the central region.

In some embodiments, the core nucleic acid strand of the nucleic acid complex described herein can comprise a first region and a second region. The first region is at the 3′ direction of the second region. The length of the core nucleic acid strand can vary in different embodiments. In some embodiments, the core nucleic acid strand comprises 20-70 linked nucleosides. For example, the core nucleic acid strand can comprise 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 linked nucleosides. In some embodiments, the core nucleic acid strand comprises 20-60 linked nucleosides. For example, the core nucleic acid strand can comprise 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60, linked nucleosides.

In some embodiments wherein a core nucleic acid strand comprises a 5′ region, a central region and a 3′ region (e.g., Design 2 in FIGS. 1 and 3), the length of the central region of the core nucleic acid strand can vary in different embodiments. In some embodiments, the central region of the core nucleic acid strand comprises 10-35 linked nucleosides. For example, the central region of the core nucleic acid strand can comprise 10, 11, 12, 13, 14, 15, 16, 17, 18, 19, 20, 21, 22, 23, 24, 25, 26, 27, 28, 29, 30, 31, 32, 33, 34, or 35 linked nucleosides.

The 3′ region and the 5′ region of the core nucleic acid strand can have a same length or a different length. The length of the 3′ region and the 5′region of the core nucleic acid strand can vary in different embodiments. In some embodiments, the length of the 3′ region and the 5′region of the core nucleic acid strand comprises 2-33 linked nucleosides. For example, the 3′ region and the 5′region of the core nucleic acid strand can comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 linked nucleosides.

The central region of the core nucleic acid strand comprises a sequence complementary to a target RNA. The length of the sequence complementary to a target RNA can vary in different embodiments. In some embodiments, the sequence complementary to a target RNA is 10-21 nucleotides in length. For example, the sequence complementary to a target RNA is 10, 11, 12, 13, 14, 15, 16, 17, 18, 19, 20, or 21 nucleotides in length.

The central region of the core nucleic acid strand comprises a sequence complementary to a passenger nucleic acid strand. The length of the sequence complementary to a passenger nucleic acid strand can vary in different embodiments. In some embodiments, the sequence complementary to a passenger nucleic acid strand is 19-25 nucleotides in length. For example, the sequence complementary to a passenger nucleic acid strand is 19, 20, 21, 22, 23, 24, or 25 nucleotides in length.

In some embodiments wherein a core nucleic acid strand comprises a first region and a second region (e.g., Design 3 in FIGS. 2-3), the length of the first region of the core nucleic acid strand can vary in different embodiments. In some embodiments, the first region of the core nucleic acid strand comprises 10-30 linked nucleosides. For example, the first region of the core nucleic acid strand can comprise 10, 11, 12, 13, 14, 15, 16, 17, 18, 19, 20, 21, 22, 23, 24, 25, 26, 27, 28, 29, or 30, linked nucleosides. In some embodiments, the first region of the core nucleic acid strand comprises 17-22 linked nucleosides.

The length of the second region of the core nucleic acid strand can vary in different embodiments. In some embodiments, the length of the second region of the core nucleic acid strand comprises 10-30 linked nucleosides. For example, the second region of the core nucleic acid strand can comprise 10, 11, 12, 13, 14, 15, 16, 17, 18, 19, 20, 21, 22, 23, 24, 25, 26, 27, 28, 29, or 30, linked nucleosides. The first region and the second region of the core nucleic acid strand can have a same length or a different length. In some embodiments, the second region of the core nucleic acid strand has a relatively short length with respect to the first region of the core nucleic acid strand. In some embodiments, the second region of the core nucleic acid strand has about 14 linked nucleosides.

The first region of the core nucleic acid strand comprises a sequence complementary to a target RNA. The length of the sequence complementary to a target RNA can vary in different embodiments. In some embodiments, the sequence complementary to a target RNA is 10-35 nucleotides in length. For example, the sequence complementary to a target RNA is 10, 11, 12, 13, 14, 15, 16, 17, 18, 19, 20, 21, 22, 23, 24, 25, 26, 27, 28, 29, 30, 31, 32, 33, 34, or 35, nucleotides in length. In some embodiments, the sequence complementary to a target RNA is 10-21 nucleotides in length.

The first region of the core nucleic acid strand comprises a sequence complementary to a passenger nucleic acid strand. The length of the sequence complementary to a passenger nucleic acid strand can vary in different embodiments. In some embodiments, the sequence complementary to a passenger nucleic acid strand is 17-22 nucleotides in length. For example, the sequence complementary to a passenger nucleic acid strand is 17, 18, 19, 20, 21, or 22 nucleotides in length. In some embodiments, the sequence of the core nucleic acid strand complementary to a passenger nucleic acid strand is about 21 nucleotides in length.

In some embodiments, the regions of a core nucleic acid strand are connected to its adjacent regions via a connector. For example, in some embodiments wherein a core nucleic acid strand comprises a 5′ region, a central region, and a 3′ region (e.g., Design 2 in FIGS. 1 and 3), the central region of the core nucleic acid strand is linked to the 5′ region and the 3′ region of the core nucleic acid strand via a connector. For example, the central region of the core nucleic acid strand is linked the 5′ region of the core nucleic acid strand via a 5′ connector. In some embodiments, the central region of the core nucleic acid strand is linked to the 3′ region of the core nucleic acid strand via a 3′ connector.

In some embodiments wherein a core nucleic acid strand comprises a first region and a second region (e.g., Design 3 in FIGS. 2-3), the first region of the core nucleic acid strand is linked to the second region of the core nucleic acid strand via a connector. For example, the first region of the core nucleic acid strand is linked the second region of the core nucleic acid strand via a 5′ connector. In some embodiments, the core nucleic acid strand only comprises one connector (e.g., 5′ connector) and does not comprise a 3′ connector.

The 5′ connector and/or 3′ connector can comprise a three-carbon linker (C3 linker), a nucleotide, any modified nucleotide described herein, or any moiety that can resist exonuclease cleavage when the core nucleic acid strand is single-stranded (e.g., after displacement of the sensor nucleic acid strand from the core nucleic acid strand). For example, the 5′ connector and/or the 3′ connector can comprise a 2′-F nucleotide such as 2′-F-adenosine, 2′-F-guanosine, 2′-F-uridine, or 2′-F-cytidine. The 5′ connector and/or the 3′ connector can comprise a 2′-O-methyl nucleotide such as 2′-O-methyladenosine, 2′-O-methylguanosine, 2′-O-methyluridine, or 2′-O-methylcytidine. The 5′ connector and/or the 3′ connector can comprise a naturally occurring nucleotide such as cytidine, uridine, adenosine, or guanosine. The 5′ connector and/or the 3′ connector of the core nucleic acid strand can comprise a phosphodiester linkage (phosphodiester 5′ and 3′ connection) cleavable by an exonuclease when in a single-stranded form. The 5′ connector and/or the 3′ connector of the core nucleic acid strand can comprise any suitable moiety that can resist exonuclease cleavage when in single-stranded form. In some embodiments, the 5′ connector of the core nucleic acid strand comprises no linker molecule except for the normal phosphodiester linkage connecting two adjacent nucleosides (see e.g., Design 3 in FIGS. 2-3).

In some embodiments, the 5′ connector can comprise or is, a C3 3-carbon linker, a nucleotide, a modified nucleotide (2′-O-methyl nucleotide, 2′-F nucleotide), a nucleotide with a phosphodiester 5′ and 3′ connection cleavable by an exonuclease when in a single stranded form, or a combination thereof. In some embodiments, the 5′ connector can comprise or is a 2′-O-methyl nucleotide such as 2′-O-methyladenosine, 2′-O-methylguanosine, 2′-O-methyluridine, or 2′-O-methylcytidine. In some embodiments, the 5′ connector can comprise or is 2′-F nucleotide such as 2′-F-adenosine, 2′-F-guanosine, 2′-F-uridine, or 2′-F-cytidine.

In some embodiments, the 3′ connector comprises or is, a C3 3-carbon linker, a nucleotide, a modified nucleotide, an exonuclease cleavage-resistant moiety when in a single stranded form, or a combination thereof. In some embodiments, the 3′ connector can comprise or is a 2′-O-methyl nucleotide such as 2′-O-methyladenosine, 2′-O-methylguanosine, 2′-O-methyluridine, or 2′-O-methylcytidine. In some embodiments, the 3′ connector comprises or is a 2′-O-methyl nucleotide such as 2′-O-methyladenosine, 2′-O-methylguanosine, 2′-O-methyluridine, or 2′-O-methylcytidine and the 5′ connector comprises or is a 2′-O-methyl nucleotide such as 2′-O-methyladenosine, 2′-O-methylguanosine, 2′-O-methyluridine, or 2′-O-methylcytidine

In some embodiments, the 5′ connector of the core nucleic acid strand does not comprise or is not a C3 3-carbon linker. In some embodiments, the 3′ connector of the core nucleic acid strand comprises or is a C3 3-carbon linker. In some embodiments, it is advantageous to not have a C3 3-carbon linker as the 5′ connector. In some embodiments, it is advantageous to have a C3 3-carbon linker as the 3′ connector. In some embodiments, the 5′ connector of the core nucleic acid strand does not comprise or is not a C3 3-carbon linker, while the 3′ connector of the core nucleic acid strand comprises or is a C3 3-carbon linker.

In some embodiments, a nucleic acid complex not having a C3 3-carbon linker as the 5′ connector exhibit higher RNA interfering activity. The nucleic acid complex can have a modified nucleotide or a nucleotide as the 5′ connector. The nucleic acid complex can have no 5′ connector. The nucleic acid complex can have a C3 3-carbon linker, a modified nucleotide, or a nucleotide as the 3′ connector. The nucleic acid complex can have no 3′ connector. In some embodiments, not having a C3 3-carbon linker as the 5′ connector increases RNA interfering activity of the nucleic acid complex by at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these value, greater than nucleic acid complexes having a C3 3-carbon linker as the 5′ connector.

In some embodiments, a nucleic acid complex having a C3 3-carbon linker as the 3′ connector exhibit higher RNA interfering activity. The nucleic acid complex can have a modified nucleotide or a nucleotide as the 5′ connector. The nucleic acid complex can have no 5′ connector. The nucleic acid complex does not have a C3 3-carbon linker as the 5′ connector. In some embodiments, having a C3 3-carbon linker as the 3′ connector increases RNA interfering activity of the nucleic acid complex by at least about 2-fold, 3-fold, 4-fold, 5-fold, 6-fold, 7-fold, 8-fold, 9-fold, 10-fold, 15-fold, 20-fold, or a number or a range between any of these value, greater than nucleic acid complexes having a modified nucleotide (e.g., 2′-O-methyl nucleotide) as the 3′ connector. In some embodiments, having a C3 3-carbon linker as the 3′ connector increases RNA interfering activity of the nucleic acid complex by at least about 2-fold, 3-fold, 4-fold, 5-fold, 6-fold, 7-fold, 8-fold, 9-fold, 10-fold, 15-fold, 20-fold, or a number or a range between any of these value, greater than nucleic acid complexes having no 3′ connector.

In some embodiments, the core nucleic acid strand do not comprise a 5′ connector and/or a 3′ connector. Instead, different regions of the core nucleic acid strand are linked to their adjacent regions via a standard phosphodiester linkage.

For example, in some embodiments wherein a core nucleic acid strand comprises a 5′ region, a central region and a 3′ region (e.g., Design 2 in FIGS. 1 and 3), the central region of the core nucleic acid strand is linked the 3′ region and/or the 5′ region via a standard phosphodiester linkage. In some embodiments, the central region of the core nucleic acid strand is linked to the 5′ region of the core nucleic acid strand via a phosphodiester linkage. In some embodiments, the central region of the core nucleic acid strand is linked to the 3′ region of the core nucleic acid strand via a phosphodiester linkage. In some embodiments, the central region of the core nucleic acid strand is linked to the 3′ region of the core nucleic acid strand via a phosphodiester linkage, while the central region of the core nucleic acid strand is linked to the 5′ region of the core nucleic acid strand via a 2′-O-methyl nucleotide such as 2′-O-methyladenosine, 2′-O-methylguanosine, 2′-O-methyluridine, or 2′-O-methylcytidine. In some embodiments, the central region of the core nucleic acid strand is linked to the 5′ region of the core nucleic acid strand via a phosphodiester linkage, while the central region of the core nucleic acid strand is linked to the 3′ region of the core nucleic acid strand via a 2′-O-methyl nucleotide such as 2′-O-methyladenosine, 2′-O-methylguanosine, 2′-O-methyluridine, or 2′-O-methylcytidine. In some embodiments, the central region of the core nucleic acid strand is linked to the 3′ region and the 5′ region of the core nucleic acid strand both via a phosphodiester linkage.

In some embodiments wherein a core nucleic acid strand comprises a first region and a second region (e.g., Design 3 in FIGS. 2-3), the first region of the core nucleic acid strand is linked to the second region via a standard phosphodiester linkage connecting two adjacent nucleosides.

In some embodiments, not having a 5′ connector and/or a 3′ connector increases RNA interfering activity of the nucleic acid complex by at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or a number or a range between any of these value, greater than nucleic acid complexes having a C3 3-carbon linker as the 5′ connector.

In some embodiments, a core nucleic acid strand can have an overhang. The overhang can be at the 3′ terminus of the core nucleic acid strand (3′ overhang). In some embodiments, the core nucleic acid strand can have a short overhang at the 3′ terminus (e.g., 1-3 nucleosides), but the 3′ overhang does not extend back into the middle of the sensor duplex to bind with the sensor nucleic acid strand (see e.g., Design 3 in FIGS. 2-3). The length of the overhang can vary in different embodiments. In some embodiments, the 3′ overhang is about one to three nucleotides in length. For example, the 3′ overhang can be one, two or three nucleotides in length. The overhang can comprise one or more modified nucleotides, such as 2′-O-methyl nucleotides. For example, the 3′ overhang can comprise two 2′-O-methyl nucleotides (see e.g., Design 3 in FIGS. 2-3). The overhang can comprise modified internucleoside linkages, such as phosphorothioate internucleoside linkages. In some embodiments, all of the nucleotides in the overhang are chemically modified. In some embodiments, all of internucleoside linkages in the 3′ overhang of the core nucleic acid strand are phosphorothioate internucleoside linkages.

### Passenger Nucleic Acid Strand

The passenger nucleic acid strand of the nucleic acid complex described herein is complementary bound to a region of the core nucleic acid strand to form a RNAi duplex (e.g., a first nucleic acid duplex). In some embodiments, the core nucleic acid strand comprises a sequence complementary to a target nucleic acid strand. In some embodiments, the passenger nucleic acid strand of the nucleic acid complex can comprise a sequence homologous to the target nucleic acid strand.

As used herein, the term “homologous” or “homology” refers to sequence identity between at least two sequences. The term “sequence identity” or “identity” in the context of two nucleic acid or polypeptide sequences makes reference to the nucleotide bases or residues in the two sequences that are the same when aligned for maximum correspondence over a specified comparison window.

In some embodiments, the sequence identity between a passenger nucleic acid strand and a target nucleic acid or a portion there of can be, be about, be at least, or be at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values. The passenger nucleic acid strand of a nucleic acid complex can have a sequence substantially identical, e.g., at least 80%, 90%, or 100%, to a target nucleic acid or a portion thereof.

The length of the passenger nucleic acid strand can vary in different embodiments. In some embodiments, the passenger nucleic acid strand comprises 10-35 linked nucleosides. For example, the core nucleic acid strand can comprise 10, 11, 12, 13, 14, 15, 16, 17, 18, 19, 20, 21, 22, 23, 24, 25, 26, 27, 28, 29, 30, 31, 32, 33, 34, or 35 linked nucleosides. In some embodiments, the passenger nucleic acid strand comprises 17-21 linked nucleosides.

In some embodiments, the passenger nucleic acid strand has a 3′ overhang, a 5′ overhang, or both in the RNAi duplex. In some embodiments, the passenger nucleic acid strand has a 3′ overhang, and the 3′ overhang is one to five nucleosides in length.

In some embodiments, the overhang of the passenger nucleic acid strand is capable of binding to the input nucleic acid strand to form a toehold, thereby initiating a toehold mediated strand displacement and causing the displacement of the passenger nucleic acid strand from the core nucleic acid strand.

In some embodiments, the overhang of the passenger nucleic acid strand is 5 to 20 nucleosides in length. For example, the overhang of the passenger nucleic acid strand can be 5, 6, 7, 8, 9, 10, 11, 12, 13, 14, 15, 16, 17, 18, 19, or 20 nucleosides in length. In some embodiments, the overhang of the passenger nucleic acid strand is 9 nucleosides in length.

In some embodiments, one or more internucleoside linages of the overhang of the passenger nucleic acid strand are phosphorothioate internucleoside linkage which can protect the overhang from degradation. In some embodiments, all internucleoside linages of the overhang of the passenger nucleic acid strand can be phosphorothioate internucleoside linkage.

In some embodiments, the passenger nucleic acid strand is fully complementary o the first region of the core nucleic acid strand, thereby forming no overhang at the 5′ and 3′ termini of the passenger nucleic acid strand in the RNAi duplex. Therefore, in some embodiments, the passenger nucleic acid strand does not have a 3′ overhang, a 5′ overhang, or both in the RNAi duplex. In some embodiments, having a blunt end with no overhang can render the passenger nucleic acid strand unfavorable for Dicer binding, thereby bypassing the Dicer-mediated cleavage.

In some embodiments, the passenger nucleic acid strand is attached to a terminal moiety and/or a blocking moiety. Any suitable terminal moiety described herein that is capable of blocking the passenger nucleic acid strand from interacting with a RNAi pathway enzyme (e.g., Dicer, RISC) can be used. The blocking moiety can include one or more suitable terminal linkers or modifications such as a blocker that can protect a single-stranded nucleic acid from nuclease degradation such as an exonuclease blocking moiety. Examples of suitable blocking moieties include, but are not limited to, a dye (e.g., fluorophore, Cy3, a dark quencher), inverted dT, a linker to link the oligonucleotide with another molecule or a particular surface (biotins, amino-modifiers, alkynes, thiol modifiers, azide, N-Hydroxysuccinimide, and cholesterol), a space (e.g., C3 spacer, Spacer 9, Spacer 18, dSpacer, tri-ethylene glycol spacer, hexa-ethylene glycol spacer), a fatty acid, one or more modified nucleotides (e.g., 2′-O-methyl, 2′-F, PS backbone connection, LNA, and/or 2′-4′ bridged base) or a combination thereof. In some embodiments, the 5′ terminus of the passenger nucleic acid is attached to an inverted-dT, a tri-ethylene-glycol, or a fluorophore. For example, a fluorophore can be attached to the 5′ terminus of the passenger nucleic acid strand via a phosphorothioate linkage.

### Sensor Nucleic Acid Strand

The sensor nucleic acid strand of the nucleic acid complex described herein comprises a region complementarily bound to at least a region of the core nucleic acid strand to form a sensor duplex (e.g., a second nucleic acid duplex). For example, the sensor nucleic acid strand can comprise a region complementarily bound to the 5′ region and the 3′ region of a core nucleic acid strand (e.g., in Design 2 of FIGS. 1 and 3). In another example, the sensor nucleic acid strand can comprise a region complementarily bound to the second region of a core nucleic acid strand (e.g., in Design 3 of FIGS. 2-3).

The length of the region complementarily bound to a core nucleic acid strand can vary in different embodiments. In some embodiments, the region complementarily bound to a core nucleic acid strand comprises 10-35 linked nucleosides. For example, the region in the sensor nucleic acid strand complementarily bound to a core nucleic acid strand can comprise 10, 11, 12, 13, 14, 15, 16, 17, 18, 19, 20, 21, 22, 23, 24, 25, 26, 27, 28, 29, 30, 31, 32, 33, 34, or 35 linked nucleosides. In some embodiments, the region complementarily bound to a core nucleic acid strand comprises 10-30 linked nucleosides. In some embodiments, the region in the sensor nucleic acid strand complementarily bound to a core nucleic acid strand comprise about 14 linked nucleosides.

The sensor nucleic acid strand can comprise a toehold (overhang). The overhang can be at the 3′ end or 5′ end, or both, of the sensor nucleic acid strand. For example, the overhang can be at the 3′ of the sensor region complementary to the core nucleic acid strand. The overhang is not complementary to the core nucleic acid strand and is capable of binding to an input nucleic acid strand, thereby initiating a toehold mediated strand displacement and causing the displacement of the passenger nucleic acid strand from the core nucleic acid strand.

The length of the overhang in the sensor nucleic acid strand can vary in different embodiments. In some embodiments, the length of the overhang can be 5-20 linked nucleotides. For example, the length of the overhang in the sensor nucleic acid strand can comprise 5, 6, 7, 8, 9, 10, 11, 12, 13, 14, 15, 16, 17, 18, 19, or 20 nucleotides in length. In some embodiments, the overhang of the sensor nucleic acid strand is 12 nucleotides in length.

The overhang of the sensor nucleic acid strand can comprise nucleotide modification introduced to improve the base-pairing affinity, nuclease resistance of the singled-stranded overhang, and thermodynamic stability to avoid spurious exonuclease induced activation of the strand. Exemplary modifications include, but not limited to, 2′-O-methyl modification, 2′-Fluoro modifications, phosphorothioate internucleoside linkages, inclusions of LNA, and the like that are identifiable by a skilled person. In some embodiments, at least 50% of the internucleoside linkages in the overhang of the sensor nucleic acid strand are phosphorothioate internucleoside linkages. For example, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two values, of the internucleoside linkages in the overhang of the sensor nucleic acid strand are phosphorothioate internucleoside linkages. In some embodiments, all internucleoside linkages in the overhang of the sensor nucleic acid strand are phosphorothioate internucleoside linkages.

In some embodiments, the 5′ terminus and/or the 3′ terminus of the sensor nucleic acid strand can comprise a terminal moiety. Any suitable terminal moiety described herein can be used. In some embodiments, the terminal moiety can include a tri- or hexa-ethylene glycol spacer, a C3 spacer, an inverted dT, an amine linker, a ligand (e.g., a targeting ligand), a fluorophore, an exonuclease, a fatty acid, a Cy3, an inverted dT attached to a tri-ethylene glycol, or a combination thereof. In some embodiments, the 3′ terminus of the sensor nucleic acid strand can be attached to a delivery ligand, a dye (e.g., fluorophore), or exonuclease. The 5′ terminus can be attached to a fatty acid, a dye (e.g., Cy3), an inverted dT, a tri-ethylene glycol, or an inverted dT attached to a tri-ethylene glycol. The delivery ligand attached to the 3′ terminus can be any suitable ligand for use in targeting the nucleic acid complex to specific cell types described elsewhere in the present disclosure. In some embodiments, the delivery ligand is a palmitic acid. In some embodiments, the palmitic acid is attached to the 3′ terminus of the sensor nucleic acid strand. In some embodiments, a nucleic acid complex construct comprising a sensor nucleic acid strand with a palmitic acid attached to the 3′ terminus of the sensor nucleic acid can achieve a higher degree of inhibition of a target nucleic acid in comparison to corresponding constructs without a 3′ terminal palmitic acid.

The sequence of the sensor nucleic acid strand can be designed to sense an input nucleic acid strand or a portion thereof. For example, from the sequence of an input biomarker, a list of all possible sensor segments which are antisense to the input strand can be generated. The sensor sequences for uniqueness in the transcriptome of the target animal can be ranked using NCBI BLAST. For human cancer cell lines, sequences can be checked against human transcript and genomic collection using the BLASTn algorithm. In some embodiments, sensor segments that have more than 17 bases of sequence complementarity and complete overhang complementarity to known or predicted RNA transcripts may be eliminated. Examples of design features of the sensor nucleic acid strand that can be used in the nucleic acid complexes described herein are described, for example, in the internatioanl patent application published as WO/2020/033938, the content of which is incorporated herein by reference.

### Input Nucleic Acid Strand

The input nucleic acid strand described herein acts as a trigger to activate (switch on) the RNA interfering activity of the nucleic acid complex (e.g., RNAi duplex) upon binding to a sequence of the sensor nucleic acid in the nucleic acid complex.

The input nucleic acid strand comprises a sequence complementary to a sequence in the sensor nucleic acid of the nucleic acid complex. The complementary binding between the input nucleic acid strand and the sensor nucleic acid strand (e.g., an overhang) causes displacement of the sensor nucleic acid strand from the core nucleic acid strand, thereby activating the RNA interfering activity of the RNAi duplex formed by the passenger nucleic acid strand and the central region of the core nucleic acid strand.

The input nucleic acid strand can be cellular RNA transcripts that are present at relatively high expression levels in a set of target cells (e.g., CNS cells) and at a relatively low level of expression in a set of non-target cells. In some embodiments, the nucleic acid complex herein described is activated (switched on) in target cells. While in the non-target cells, the nucleic acid complex remains inactivated (switched off). In some embodiments, the target cells are central nervous system cells.

In some embodiments, in the target cells, the input nucleic acid strand is expressed at a level of, about, at least, or at least about 2-fold, 5-fold, 10-fold, 20-fold, 30-fold, 40-fold, 50-fold, 60-fold, 70-fold, 80-fold, 90-fold, or 100-fold higher than in the non-target cells. In some embodiments, in the target cells, the input nucleic acid strand is expressed at a level of, about, at least, at least about 50, 100, 200, 300, 400, 500, 600, 700, 800, 900, 1000 transcripts. In some embodiments, in the non-target cells, the input nucleic acid strand is expressed at a level of less than 50, less than 40, less than 30, less than 20, or less than 10 transcripts. Preferably, the non-target cells have no detectable expression of the input nucleic acid strand.

The input nucleic acid strand can comprise an mRNA, an miRNA, or a non-coding RNA such as a long non-coding RNA, an RNA fragment, or an RNA transcript of a virus. In some embodiments, the input nucleic acid strand is an RNA transcript that is expressed in a set of cells that are causing the progression of a disease and are therefore targeted for RNAi therapy. The non-target cells are usually a set of cells where silencing of a target RNA can cause side effects that are not beneficial for therapy. For treating a disease or a condition where the input RNA is overexpressed in target cells, the nucleic acid complex can be designed such that the sensor nucleic acid strand comprises a sequence complementary to the input RNA sequence. Upon administration of the nucleic acid complex, the binding of sensor nucleic acid strand to the input RNA induces the dissociation of the RNAi duplex from the sensor duplex in target cells thereby to activate the RNAi targeting the disease or condition.

In some embodiments, the input nucleic acid strand comprises a biomarker. The term “biomarker” used herein refers to a nucleic acid sequence (DNA or RNA) that is an indicator of a disease or disorder, a susceptibility to a disease or disorder, and/or of response to therapeutic or other intervention. A biomarker can reflect an expression, function or regulation of a gene. The input nucleic acid strand can comprise any disease biomarker known in the art. In some embodiments, the input nucleic acid strand can comprise any disease biomarker related to a neurological disease or disorder such as a disease or disorder of the central nervous system (CNS).

In some embodiments, the input nucleic acid strand is a mRNA. The input nucleic acid strand can be a cell type or cell-state specific or selective mRNA such as mRNAs specific for cells of the central nervous system, including but not limited to C3, GFAP, IBA-1, NPPA, CSF1R, SLC1A2, PLP1, MBP, amiloride sensitive cation channel 3 (ACCN3), SCN10A (Nav1.8 sodium ion channel), Edg7 (endothelial differentiation, GPCR, lysophosphatidic acid receptor 3), HTR3B (serotonin receptor 3b), HTR3A (serotonin receptor 3a), GPR64 (GPCR), NTRK1 (NGF receptor, receptor tyrosine kinase), CHRNA6 (component of AChR), P2RX3 (ATP gated ion channel), KCNK18 (potassium ion channel), GAL (galanin and GMAP prepropeptide), PRPH (peripherin, cytoskeletal protein), CALCA (calcitonin hormone), CALCB (calcitonin peptide B), P2RX3 (ATP gated ion channel), and NAV1.7 (sodium ion channel).

In some embodiments, the input nucleic acid strand is a universal mRNA that is not cell type specific or selective. Examples of universal mRNA include, but are not limited to, miR-133b, miR218-2, miR-15b, miR101-1, miR107, miR-335, miR-345, miR-146a, miR-197, miR-320, miR-423, miR-511, miR-1, miR-22, miR-29a, miR-330, miR-132, miR-196, miR-486, miR-100, miR-151-3p, miR-16, miR-219-2-3p, miR-27b, miR-451, miR-92a, miR-34b, miR-338-3p, miR-27a, miR-155, miR-146a, miR-32-3p, miR-146, miR-524-5p miR-582-3p, miR-24-2, miR-142-3p, miR-142-5p, miR-1461, miR-146b, mir-21-5p, mir-23a-3p, mir-29c-3p, mir-29b-3p, mir-124-3p, and 5.8s ribosomal RNA. In some embodiments, the input nucleic acid strand comprises mir-21-5p, mir-23a-3p, mir-29c-3p, mir-29b-3p, mir-124-3p, and 5.8s ribosomal RNA. In some embodiments, the input nucleic acid strand is a non-coding RNA, for example MALAT1 (metastasis associated lung adenocarcinoma transcript 1, also known as NEAT2 (noncoding nuclear-enriched abundant transcript 2).

In some embodiments, nucleic acid complexes herein described that are designed to bind a universal input nucleic acid can result in improved distribution, deeper knockdown, potent and long-duration inhibition of difficult CNS targets. In some embodiments, nucleic acid complexes herein described that are designed to bind a cell-selective input nucleic acid can result in region (striatum) or cell-type restricted (e.g., motor neuron) inhibition to reduce side effect and can target specific cell types (e.g., astroglia) or states (e.g., reactive states) to disentangle pleiotropic effects.

### Target RNA

The core nucleic acid strand comprises a sequence complementary to a target RNA in order to direct target-specific RNA interference. In some embodiments, the target RNA is a cellular RNA transcript. The target RNA can be an mRNA, an miRNA, a non-coding RNA, a viral RNA transcript, or a combination thereof.

As used herein, a “target RNA” refers to a RNA whose expression is to be selectively inhibited or silenced through RNA interference. A target RNA can be a target gene comprising any cellular gene or gene fragment whose expression or activity is associated with a disease, a disorder or a condition. In some embodiments, the target RNA is a target gene whose expression or activity is associated with a neurological disease or disorder such as a disease or disorder of the central nervous system. A target RNA can also be a foreign or exogenous RNA or RNA fragment whose expression or activity is associated with a disease, a disorder or a certain condition (e.g., a viral RNA transcript or a pro-viral gene).

Exemplary genes associated with a neurological disease or disorder (e.g., a CNS disease or a condition) include, but are not limited to, HTT (Huntingtin gene), APP, ALS2, SETX, SMN, Rab7, α-COP, TDP-43, VAPB, IT15, DRPLA, SCA3/MJD, SNCA, SPR, DJ1, MAPT, SOD1, PSEN1, PSEN2, C9orf72, GRN, SNCA, PRKN, PINK1, PARK7, LRRK2, VPS35, FUS, TARDBP, UBQLN2, BACE1, CASP3, TGM2, NFE2L3, ADRB1, CAMK2A, CBLN1, CDK5R1, GABRA1, MAPK10, NOS1, NPTX2, NRGN, NTS, PDCD2, PDE4D, PENK, SYT1, TTR, LRDD, CYBA, ATF3, ATF6, CASP2, CASP1, CASP7, CASP8, CASP9, HRK, C1QBP, BNIP3, MAPK8, MAPK14, Rac1, GSK3B, P2RX7, TRPM2, PARG, CD38, STEAP4, BMP2, GJA1, TYROBP, CTGF, ANXA2, RHOA, DUOX1, RTP801, RTP801L, NOX4, NOX1, NOX2 (gp91pho, CYBB), NOX5, DUOX2, NOXO1, NOXO2 (p47phox, NCF1), NOXA1, NOXA2 (p67phox, NCF2), p53 (TP53), HTRA2, KEAP1, SHC1, ZNHIT1, LGALS3, HI95, SOX9, ASPP1, ASPP2, CTSD, CAPNS1, FAS and FASLG, NOGO and NOGO-R; TLR1, TLR2, TLR3, TLR4, TLR6, TLR7, TLR8, TLR9, IL1bR, MYD88, TICAM, TIRAP, HSP47, and others apparent to a person skilled in the art.

In some embodiments, the exemplary genes associated with a CNS disease or a condition include HTT, C3, GFAP, IBA-1, NPPA, CSF1R, SLC1A2, PLP1, MBP, amiloride sensitive cation channel 3 (ACCN3), SCN10A (Nav1.8 sodium ion channel), Edg7 (endothelial differentiation, GPCR, lysophosphatidic acid receptor 3), HTR3B (serotonin receptor 3b), HTR3A (serotonin receptor 3a), GPR64 (GPCR), NTRK1 (NGF receptor, receptor tyrosine kinase), CHRNA6 (component of AChR), P2RX3 (ATP gated ion channel), KCNK18 (potassium ion channel), GAL (galanin and GMAP prepropeptide), PRPH (peripherin, cytoskeletal protein), CALCA (calcitonin hormone), CALCB (calcitonin peptide B), P2RX3 (ATP gated ion channel), and NAV1.7 (sodium ion channel).

### Chemical Modification

The nucleic acid strands (the core nucleic acid strand, the passenger nucleic acid strand, and/or the sensor nucleic acid strand) comprised in the nucleic acid complexes herein described can be a non-standard, modified nucleic acid strand comprising non-standard, modified nucleotides (nucleotide analog) or non-standard, modified nucleosides (nucleoside analog). The term “nucleotide analog” or “modified nucleotide” refers to a non-standard nucleotide comprising one or more modifications (e.g., chemical modifications), including non-naturally occurring ribonucleotides or deoxyribonucleotides. The term “nucleoside analog” or “modified nucleoside” refers to a non-standard nucleoside comprising one or more modification (e.g., chemical modification), including non-naturally occurring nucleosides other than cytidine, uridine, adenosine, guanosine, and thymidine. The modified nucleoside can be a modified nucleotide without a phosphate group. The chemical modifications can include replacement of one or more atoms or moieties with a different atom or a different moiety or functional group (e.g., methyl group, and hydroxyl group).

The modifications are introduced to alter certain chemical properties of the nucleotide/nucleoside such as to increase or decrease thermodynamic stability, to increase resistance to nuclease degradation (e.g., exonuclease resistant), and/or to increase binding specificity and minimize off-target effects. For example, thermodynamic stability can be determined based on measurement of melting temperature Tₘ. A higher Tₘ can be associated with a more thermodynamically stable chemical entity.

In some embodiments, the modification can render one or more of the nucleic acid strands in the nucleic acid complex to resist exonuclease degradation/cleavage. The term “exonuclease” as used herein, indicates a type of enzyme that works by cleaving nucleotides one at a time from the end (exo) of a polynucleotide chain. A hydrolyzing reaction that breaks phosphodiester bonds at either the 3′ or the 5′ end occurs. A 3′ and 5′ exonuclease can degrade RNA and DNA in cells, and can degrade RNA and DNA in the interstitial space between cells and in plasma, with a high efficiency and a fast kinetic rate. A close relative is the endonuclease, which cleaves phosphodiester bonds in the middle (endo) of a polynucleotide chain. 3′ and 5′ exonuclease and exonucleolytic complexes can degrade RNA and DNA in cells, and can degrade RNA and DNA in the interstitial space between cells and in plasma. The term “exoribonuclease” as used herein, refers to exonuclease ribonucleases, which are enzymes that degrade RNA by removing terminal nucleotides from either the 5′ end or the 3′ end of the RNA molecule. Enzymes that remove nucleotides from the 5′ end are called 5′-3′ exoribonucleases, and enzymes that remove nucleotides from the 3′ end are called 3′-5′ exoribonucleases.

The modification can comprise phosphonate modification, ribose modification (in the sugar portion), and/or base modification.

In some embodiments, the modified nucleotide can comprise modifications to the sugar portion of the nucleotides. For example, the 2′ OH-group of a nucleotide can be replaced by a group selected from H, OR, R, F, Cl, Br, I, SH, SR, NH₂, NHR, NR₂, COOR, or OR, wherein R is substituted or unsubstituted C1-C6 alkyl, alkenyl, alkynyl, aryl, etc. In some embodiments, the 2′ OH-group of a nucleotide or nucleoside is replaced by 2′ O-methyl group and the modified nucleotide or nucleoside is a 2′-O-methyl nucleotide or 2′-O-methyl nucleoside (2′-OMe). The 2′-O-methyl nucleotide or 2′-O-methyl nucleoside can be 2′-O-methyladenosine, 2′-O-methylguanosine, 2′-O-methyluridine, or 2′-O-methylcytidine. In some embodiments, the 2′ OH-group of a nucleotide is replaced by fluorine (F), and the modified nucleotide or nucleoside is a 2′-F nucleotide or 2′-F nucleoside (2′-deoxy-2′-fluoro or 2′-F). The 2′-F nucleotide or 2′-F nucleoside can be 2′-F-adenosine, 2′-F-guanosine, 2′-F-uridine, or 2′-F-cytidine. The modifications can also include other modifications such as nucleoside analog phosphoramidites. In some embodiments, glycol nucleic acids can be used.

In some embodiments, the modified nucleotide can comprise a modification in the phosphate group of the nucleotide, e.g., by substituting one or more of the oxygens of the phosphate group with sulfur or a methyl group. In some embodiments, one or more of the nonbridging oxygens of the phosphate group of a nucleotide is replaced by a sulfur.

In some embodiments, the nucleic acid strands herein described comprise one or more non-standard internucleoside linkage that is not a phosphodiester linkage. In some embodiments, the nucleic acid strands herein described comprise one or more phosphorothioate internucleoside linkages. The term “phosphorothioate linkage” (PS) as used herein, indicates a bond between nucleotides in which one of the nonbridging oxygens is replaced by a sulfur. In some embodiments, both nonbridging oxygens may be replaced by a sulfur (PS2). In some embodiments, one of the nonbridging oxygens may be replaced by a methyl group. The term “phosphodiester linkage” as described herein indicates the normal sugar phosphate backbone linkage in DNA and RNA wherein a phosphate bridges the two sugars. In some embodiments, the introduction of one or more phosphorothioate linkage in the core nucleic acid strand, the passenger nucleic acid strand, and/or the sensor nucleic acid strand can endow the modified nucleotides with increased resistance to nucleases (e.g., endonucleases and/or exonucleases).

In some embodiments, the modified nucleotide can comprise modifications to or substitution of the base portion of a nucleotide. For example, uridine and cytidine residues can be substituted with pseudouridine, 2-thiouridine, N6-methyladenosine, 5-methycytidine or other base analogs of uridine and cytidine residues. Adenosine can comprise modifications to Hoogsteen (e.g., 7-triazolo-8-aza-7-deazaadenosines) and/or Watson-Crick face of adenosine (e.g., N2-alkyl-2-aminopurines). Examples of adenosine analogs also include Hoogsteen or Watson-Crick face-localized N-ethylpiperidine triazole-modified adenosine analogs, N-ethylpiperidine 7-EAA triazole (e.g., 7-EAA, 7-ethynyl-8-aza-7-deazaadenosine) and other adenosine analogs identifiable to a person skilled in the art. Cytosine may be substituted with any suitable cytosine analogs identifiable to a person skilled in the art. For example, cytosine can be substituted with 6′-phenylpyrrolocytosine (PhpC) which has shown comparable base pairing fidelity, thermal stability and high fluorescence.

In some embodiments, one or more nucleotides in the nucleic acid complex disclosed herein can be substituted with a universal base. The term “universal base” refers to nucleotide analogs that form base pairs with each of the natural nucleotides with little discrimination between them. Examples of universal bases include, but are not limited to, hypoxanthine and derivatives thereof, inosine and derivatives thereof, azole carboxamide and derivatives thereof, nitroazole and derivatives thereof (e.g., 3-nitropyrrole, 4-nitroindole, 5-nitroindole, 6-nitroindole, nitroimidazole, and 4-nitropyrazole), phenyl C-ribonucleoside and derivatives thereof, naphthyl C-ribonucleoside and derivatives thereof, and other aromatic derivatives, or a combination thereof. In some embodiments, the universal bases comprised in the nucleic acid complex herein described comprise inosine or analogues thereof. Analogues of inosine include, for example, 2′-deoxyisoinosine, 7-deaza-2′-deoxyinosine, and 2-aza-2′-deoxyinosine. Examples of universal base and analogues thereof are described, for example, in Loakes, 2001, Nucleic Acids Research, 29, 2437-2447, the content of which is incorporated by reference in its entirety.

In some embodiments, base modification disclosed herein can reduce innate immune recognition while making the nucleic acid complex more resistant to nucleases. Examples of base modifications that can be used in the nucleic acid complex disclosed herein are also described, for example, in Hu et al. (Signal Transduction and targeted Therapy 5:101 (2020)), the content of which is incorporated by reference in its entirety. In some embodiments, the base modification disclosed herein (e.g., universal base) can result in reduced base pairing strength, thus decreasing the thermodynamics stability and the melting temperature of a formed duplex (e.g., sensor duplex).

In some embodiments, the nucleic acid strands (the core nucleic acid strand, the passenger nucleic acid strand, and/or the sensor nucleic acid strand) comprised in the nucleic acid complexes herein described can comprise one or more locked nucleic acids or analogs thereof. Exemplary locked nucleic acid analogs include, for example, their corresponding locked analog phosphoramidites and other derivatives apparent to a skilled artisan.

As used herein, the term “locked nucleic acids” (LNA) indicates a modified RNA nucleotide. The ribose moiety of an LNA nucleotide is modified with an extra bridge connecting the 2′ and 4′ carbons (a 2′-O, 4′-C methylene bridge). The bridge “locks” the ribose in the 3′-endo structural conformation and restricts the flexibility of the ribofuranose ring, thereby locking the structure into a rigid bicyclic formation. LNA nucleotides can be mixed with DNA or RNA bases in the oligonucleotide whenever desired. The incorporation of LNA into the nucleic acid complexes disclosed herein can increase the thermal stability (e.g., melting temperature), hybridization specificity of oligonucleotides as well as accuracies in allelic discrimination. LNA oligonucleotides display hybridization affinity toward complementary single-stranded RNA and complementary single- or double-stranded DNA. Additional information about LNA can be found, for example, at www.sigmaaldrich.com/technical-documents/articles/biology/locked-nucleic-acids-faq.html. In some embodiments, glycol nucleic acids can be used.

In some embodiments, the nucleic acid strands (the core nucleic acid strand, the passenger nucleic acid strand, and/or the sensor nucleic acid strand) comprised in the nucleic acid complexes herein described can comprise other chemically modified nucleotide or nucleoside with 2′-4′ bridging modifications. A 2′-4′ bridging modification refers to the introduction of a bridge connecting the 2′ and 4′ carbons of a nucleotide. The bridge can be a 2′-O, 4′-C methylene bridge (e.g., in LNA). The bridge can also be a 2′-O, 4′-C ethylene bridge (e.g., in ethylen-bridged nucleic acids (ENA)) or any other chemical linkage identifiable to a person skilled in the art.

In some embodiments, the introduction of LNA, analogues thereof, or other chemically modified nucleotides with 2′-4′ bridging modifications in the nucleic acid complex herein described can enhance hybridization stability as well as mismatch discrimination. For example, a nucleic acid complex comprising a sensor nucleic acid strand with LNA, analogues thereof, or other chemically modified nucleotides with 2′-4′ bridging modifications can have an enhanced sensitivity to distinguish between matched and mismatched input nucleic acid strand (e.g., in the complementary binding between an input nucleic acid strand and a sensor nucleic acid strand).

In some embodiments, one or more of the nucleic acid strands of the nucleic acid complex can comprise a chemical moiety linked to the 3′ and/or 5′ terminus of the strand. The terminal moiety can include one or more any suitable terminal linkers or modifications. For example, the terminal moiety can include a linker to link the oligonucleotide with another molecule or a particular surface (biotins, amino-modifiers, alkynes, thiol modifiers, azide, N-Hydroxysuccinimide, and cholesterol), a dye (e.g., fluorophore or a dark quencher), a fluorine modified ribose, a space (e.g., C3 spacer, Spacer 9, Spacer 18, dSpacer, tri-ethylene glycol spacer, hexa-ethylene glycol spacer), moieties and chemical modification involved in click chemistry (e.g., alkyne and azide moieties), and any linkers or terminal modifications that can be used to attach the 3′ and 5′ end to other chemical moieties such as antibodies, gold or other metallic nanoparticles, polymeric nanoparticles, dendrimer nanoparticles, small molecules, single chain or branched fatty acids, peptides, proteins, aptamers, and other nucleic acid strands and nucleic acid nanostructures. The terminal moiety can serve as a label capable of detection or a blocker to protect a single-stranded nucleic acid from nuclease degradation. Additional linkers and terminal modification that can be attached to the terminus of the sensor nucleic acid strand are described in www.idtdna.com/pages/products/custom-dna-rna/oligo-modifications and www.glenresearch.com/browse/labels-and-modifiers, the contents of which are incorporated herein by reference in their entirety.

Additional modifications to the nucleotides and/or nucleosides can also be introduced to one or more strands of the nucleic acid complex herein described, such as modifications described in Hu et al. (Signal Transduction and targeted Therapy 5:101 (2020)), the content of which is incorporated by reference in its entirety.

### Ribose Modification

The percentage of the modified nucleosides of the nucleic acid complex can vary in different embodiments. In some embodiments, the percentage of the modified nucleosides of the nucleic acid complex herein described can be, be about, be at least, or be at least about 80%, 85%, 90%, 95%, 99%, 100%, or a number or a range between any two of these values. In some embodiments, at least 90%, 91%, 92%, 93%, 94%, 95%, or a number or a range between any two of these values of the nucleotides of the nucleic acid complex are modified (e.g., non-DNA and non-RNA). In some embodiments, all of the nucleotides of the nucleic acid complex are modified (e.g., non-DNA and non-RNA).

The percentage of the modified nucleosides in one or more strands of the nucleic acid complex can vary in different embodiments. In some embodiments, the percentage of the modified nucleosides in a core nucleic acid strand herein described can be, be about, be at least, or be at least about 80%, 85%, 90%, 95%, 99%, 100%, or a number or a range between any two of these values. In some embodiments, all of the nucleosides of a core nucleic acid strand are chemically modified.

In some embodiments, the percentage of the modified nucleosides in the region of a core nucleic acid strand complementarily bound to a passenger nucleic acid strand (e.g., the central region in Design 1 and Design 2 of FIGS. 1 and 3 or the first region in Design 3 of FIGS. 2-3) can be, be about, be at least, or be at least about 80%, 85%, 90%, 95%, 99%, 100%, or a number or a range between any two of these values. In some embodiments, all of the nucleosides of the first region of a core nucleic acid strand are chemically modified.

In some embodiments, the percentage of the modified nucleosides in the region of a core nucleic acid strand complementarily bound to a sensor nucleic acid strand can be, be about, be at least, or be at least about 80%, 85%, 90%, 95%, or a number or a range between any two of these values.

In some embodiments wherein a core nucleic acid strand comprises a 5′ region, a central region and a 3′ region, the percentage of the modified nucleosides in the 5′ region of a core nucleic acid strand herein described can be, be about, be at least, or be at least about 80%, 85%, 90%, 95%, 99%, 100%, or a number or a range between any two of these values. In some embodiments, all of the nucleosides of the 5′ region of a core nucleic acid strand are chemically modified.

In some embodiments, the percentage of the modified nucleosides in the 3′ region of a core nucleic acid strand herein described can be, be about, be at least, or be at least about 80%, 85%, 90%, 95%, or a number or a range between any two of these values. In some embodiments, all of the nucleosides of the 3′ region of a core nucleic acid strand are chemically modified.

In some embodiments wherein a core nucleic acid strand comprises a first region and a second region, the percentage of the modified nucleosides in the first region of a core nucleic acid strand herein described can be, be about, be at least, or be at least about 50%, 60%, 70%, 80%, 85%, 90%, 95%, or a number or a range between any two of these values. In some embodiments, all of the nucleosides of the first region of a core nucleic acid strand are chemically modified.

In some embodiments, the percentage of the modified nucleosides in the second region of a core nucleic acid strand herein described can be, be about, be at least, or be at least about 50%, 60%, 70%, 80%, 85%, 90%, 95%, or a number or a range between any two of these values. In some embodiments, all of the nucleosides of the second region of a core nucleic acid strand are chemically modified. In some embodiments, the percentage of the modified nucleosides in a passenger nucleic acid strand herein described can be, be about, be at least, or be at least about 50%, 60%, 70%, 80%, 85%, 90%, 95%, or a number or a range between any two of these values. In some embodiments, all of the nucleosides of a passenger nucleic acid strand are chemically modified.

In some embodiments, the percentage of the modified nucleosides in a sensor nucleic acid strand herein described can be, be about, be at least, or be at least about 50%, 60%, 70%, 80%, 85%, 90%, 95%, or a number or a range between any two of these values. In some embodiments, all of the nucleosides of a sensor nucleic acid strand are chemically modified.

The modified nucleosides in one or more of the core nucleic acid strand, the passenger nucleic acid strand, and the sensor nucleic acid strand can comprise 2′-O-methyl nucleoside and/or 2′-F nucleoside. In some embodiments, the percentage of 2′-O-methyl nucleoside and/or 2′-F nucleoside in the nucleic acid complex herein described can be, be about, be at least, be at least about, be at most, or be at most about 10%-50%. In some embodiments, the percentage of 2′-O-methyl nucleoside and/or 2′-F nucleoside in a core nucleic acid strand herein described can be, be about, be at least, be at least about, be at most, or be at most about 10%-50%. In some embodiments, the percentage of 2′-O-methyl nucleoside and/or 2′-F nucleoside in a passenger nucleic acid strand herein described can be, be about, be at least, be at least about, be at most, or be at most about 10%-50%. In some embodiments, the percentage of 2′-O-methyl nucleoside and/or 2′-F nucleoside in a sensor nucleic acid strand herein described can be, be about, be at least, be at least about, be at most, or be at most about 10%-50%.

### Phosphate Modification

The percentage of phosphate modification to the nucleotides in the nucleic acid complex described herein can vary in different embodiments. In some embodiments, the phosphate modification comprises or is a phosphorothioate internucleoside linkage. In some embodiments, the percentage of phosphorothioate internucleoside linkages in a core nucleic acid strand is less than 5%, less than 10%, less than 25%, less than 50%, or a number or a range between any two of these values. In some embodiments, the core nucleic acid strand does not comprise a phosphorothioate internucleoside linkage modification.

In some embodiments, the percentage of phosphodiester internucleoside linkages in a core nucleic acid strand is about, at least, or at least about 50%, 80% or 95%, or a number or a range between any two of these values. In some embodiments, all the internucleoside linkages in the core nucleic acid strand are phosphodiester internucleoside linkage.

In some embodiments where a core nucleic acid strand comprises a 5′ region, a central region, and a 3′ region (e.g., Design 2 in FIGS. 1 and 3), the 5′ terminus of the central region of the core nucleic acid strand comprises at least one phosphorothioate internucleoside linkage (e.g., one, two or three phosphorothioate internucleoside linkage). In some embodiments, the 3′ terminus of the central region of the core nucleic acid strand comprises at least one phosphorothioate internucleoside linkage (e.g., one, two or three phosphorothioate internucleoside linkage). In some embodiments, each of the 5′ terminus of the central region of the core nucleic acid strand and the 3′ terminus of the central region of the core nucleic acid strand independently comprises one or more phosphorothioate internucleoside linkages (e.g., one, two or three phosphorothioate internucleoside linkage). In some embodiments, the central region of the core nucleic acid strand does not comprise phosphorothioate internucleoside linkages except for the phosphorothioate internucleoside linkage(s) between two or three nucleosides at the 5′ terminus, 3′ terminus, or both, of the central region.

In some embodiments, the internucleoside linkages between the one to three nucleotides (e.g., one, two, or three nucleotides) adjacent to the 3′ of the 5′ connector of the core nucleic acid strand are phosphorothioate internucleoside linkages. In some embodiments, the internucleoside linkages between the one or two nucleotides adjacent to the 5′ of the 3′ connector of the core nucleic acid strand are phosphorothioate internucleoside linkages. In some embodiments, the internucleoside linkages between the one to three nucleotides (e.g., one, two, or three nucleotides) adjacent to the 3′ of the 3′ connector of the core nucleic acid strand are phosphorothioate internucleoside linkages. In some embodiments, the 3′ region of the core nucleic acid strand does not comprise phosphorothioate internucleoside linkages except for the phosphorothioate internucleoside linkage(s) between the one to three nucleotides (e.g., one, two, or three nucleotides) adjacent to the 3′ of the 3′ connector of the core nucleic acid strand. In some embodiments, the 5′ region of the core nucleic acid strand does not comprise phosphorothioate internucleoside linkages.

In some embodiments, where a core nucleic acid strand comprises a first region and a second region (e.g., Design 3 in FIGS. 2-3), the 3′ terminus of the first region of the core nucleic acid strand comprises at least one phosphorothioate internucleoside linkage (e.g., one, two or three phosphorothioate internucleoside linkage). The phosphorothioate internucleoside linkage can be between the last two, three, or four nucleosides at the 3′ terminus of the first region of the core nucleic acid strand. In some embodiments, the 5′ terminus of the first region of the core nucleic acid strand comprises at least one phosphorothioate internucleoside linkage (e.g., one, two or three phosphorothioate internucleoside linkage). The phosphorothioate internucleoside linkage can be between the last two, three, or four nucleosides at the 5′ terminus of the first region of the core nucleic acid strand. In some embodiments, each of the 5′ terminus of the first region of the core nucleic acid strand and the 3′ terminus of the first region of the core nucleic acid strand independently comprises one or more phosphorothioate internucleoside linkages (e.g., one, two or three phosphorothioate internucleoside linkage). In some embodiments, the first region of the core nucleic acid strand does not comprise phosphorothioate internucleoside linkages except for the phosphorothioate internucleoside linkage(s) between the last two or three nucleosides at the 5′ terminus, 3′ terminus, or both, of the first region. For example, the first region of the core nucleic acid strand does not comprise phosphorothioate internucleoside linkages except for the phosphorothioate internucleoside linkage(s) between the last three nucleosides at the 5′ terminus and the last three nucleosides 3′ terminus of the first region.

In some embodiments, the percentage of phosphorothioate internucleoside linkages in the second region of a core nucleic acid strand is less than 5%, less than 10%, or a number or a range between any two of these values. In some embodiments, the second region of a core nucleic acid strand does not comprise phosphorothioate internucleoside linkages.

In some embodiments, the passenger nucleic acid strand comprises one or more phosphorothioate internucleoside linkage. The percentage of phosphorothioate internucleoside linkages in a passenger nucleic acid strand is less than 5%, less than 10%, less than 25%, less than 50%, or a number or a range between any two of these values.

In some embodiments, the 5′ terminus of the passenger nucleic acid strand comprises at least one phosphorothioate internucleoside linkage (e.g., one, two, or three phosphorothioate internucleoside linkage). In some embodiments, the 3′ terminus of the passenger nucleic acid strand comprises at least one phosphorothioate internucleoside linkage (e.g., one, two, or three phosphorothioate internucleoside linkage). In some embodiments, the passenger nucleic acid strand does not comprise phosphorothioate internucleoside linkages except for the phosphorothioate internucleoside linkage(s) between the last two, three, or four nucleosides at the 5′ terminus, 3′ terminus, or both, of the passenger nucleic acid strand. In some embodiments, the passenger nucleic acid strand does not comprise phosphorothioate internucleoside linkages except for the phosphorothioate internucleoside linkage(s) between the last two to three nucleosides at the 5′ terminus and the last two to three nucleosides at 3′ terminus of the passenger nucleic acid strand.

In some embodiments, the sensor nucleic acid strand comprises one or more phosphorothioate internucleoside linkage. The percentage of phosphorothioate internucleoside linkages in a sensor nucleic acid strand can be less than 5%, less than 10%, less than 25%, less than 50%, less than 60%, less than 70% or a number or a range between any two of these values.

In some embodiments, the 5′ terminus of the sensor nucleic acid strand comprises at least one phosphorothioate internucleoside linkage (e.g., one, two or three phosphorothioate internucleoside linkage). In some embodiments, the 3′ terminus of the sensor nucleic acid strand comprises at least one phosphorothioate internucleoside linkage (e.g., one to twenty phosphorothioate internucleoside linkage. In some embodiments, each of the 5′ terminus of the sensor nucleic acid strand and the 3′ terminus of the sensor nucleic acid strand independently comprises one or more phosphorothioate internucleoside linkages (e.g., one, two or three at the 5′ terminus or one to twenty at the 3′ terminus). In some embodiments, the sensor nucleic acid strand does not comprise phosphorothioate internucleoside linkages except for the phosphorothioate internucleoside linkage(s) at the 5′ terminus, 3′ terminus, or both, of the sensor nucleic acid strand. In some embodiments, the phosphorothioate internucleoside linkages at the 3′ terminus of the sensor nucleic acid strand are in the singled-stranded overhang of the sensor nucleic acid strand.

### LNA, Analogues Thereof and 2′-4′Bridging Modification

The percentage of the LNA or analogues thereof of the nucleic acid complex can vary in different embodiments. In some embodiments, the percentage of the LNA or analogues thereof of the nucleic acid complex herein described can be about 10%-50%.

The percentage of the LNA or analogues thereof in one or more strands of the nucleic acid complex can vary in different embodiments. In some embodiments, the percentage of the LNA or analogues thereof in a core nucleic acid strand herein described can be, be about, be at most, or be at most about 5%, 10%, or 15%. In some embodiments, the percentage of the LNA or analogues thereof in a passenger nucleic acid strand herein described can be, be about, be at most, or be at most about 5%, 10%, or 15%. In some embodiments, a percentage of the LNA or analogues thereof in a passenger nucleic acid strand herein described greater than 5%, greater than 10%, or greater than 15% can decrease the RNAi activity of the nucleic acid complex. In some embodiments, the percentage of the LNA or analogues thereof in a sensor nucleic acid strand herein described can be, be about, be at least, be at least about, be at most, or be at most about 10%-50%.

The percentage of 2′-4′ bridging modification of the nucleic acid complex can vary in different embodiments. In some embodiments, the percentage of the 2′-4′ bridging modification of the nucleic acid complex herein described can be about 10%-50%.

### Base Modification

In some embodiments wherein a core nucleic acid strand comprises a 5′ region, a central region, and a 3′ region (e.g., Design 2 in FIGS. 1 and 3), the 5′ region and/or the 3′ region of the core nucleic acid strand can comprise one or more universal base herein described. For example, the 5′ region and/or the 3′ region of the core nucleic acid strand can comprise one or more inosine.

In some embodiments wherein a core nucleic acid strand comprises a first region and a second region (e.g., Design 3 in FIGS. 2-3), the second region of the core nucleic acid strand can comprise one or more universal base herein described. For example, the second region of the core nucleic acid strand can comprise one or more inosine.

In some embodiments, a sensor nucleic acid, and particularly the region of the sensor nucleic acid that is complementarily bound to a core nucleic acid strand, can comprise one or more universal base herein described.

### Pharmaceutical Compositions and Methods of Administration

### Compositions

Also provided herein include pharmaceutical compositions comprising the nucleic acid complex as herein described, in combination with one or more compatible and pharmaceutically acceptable carriers.

The nucleic acid complex herein described can be suitably formulated and introduced into cell environment by any means that allows for a sufficient portion of the constructs to enter the cells to induce gene silencing, if it occurs.

The nucleic acid complex can be admixed, encapsulated, conjugated, or associated with other molecules, molecule structures, mixtures of compounds or agent, or other formulations for assistance in uptake, distribution, and/or absorption during delivery.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Pharmaceutically acceptable carrier can comprise a pharmaceutical acceptable salt. As used herein, a “pharmaceutical acceptable salt” includes a salt of an acid form of one of the components of the compositions herein described. These include organic or inorganic acid salts of the amines. Preferred acid salts are the hydrochlorides, acetates, salicylates, nitrates and phosphates. Other suitable pharmaceutically acceptable salts are well known to those skill in the art and include basic salts of a variety of inorganic and organic acids.

In some embodiments, pharmaceutically acceptable salts to be used with the nucleic acid complex herein described include but are not limited to (1) salts formed with cations such as sodium, potassium, ammonium, magnesium, calcium, polyamines such as spermine and spermidine; (2) acid addition salts formed with inorganic acids, for example hydrochloric acid, hydrobromic acid, sulfuric acid, phosphoric acid, nitric acid and the like; (3) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalene disulfonic acid, polygalacturonic acid, and the like; and (4) salts formed from elemental anions such as chlorine, bromine, and iodine.

### Delivery Vesicles

Various delivery systems can be employed for delivering the nucleic acid complex herein described such as antibody conjugates, micelles, natural polysaccharides, peptides, synthetic cationic polymers, microparticles, lipid-based nanovectors among others.

Delivery systems and the related excipients used for delivery of the nucleic acid complex herein described can vary in different embodiments. Delivery systems can be selected based on the mode of administration utilized, types of formulations, target sites, and types of diseases or disorders to be treated to facilitate tissue penetration, cellular uptake and to prevent extravasation and endosomal escape.

In some embodiments, the nucleic acid complex can be formulated with one or more polymers to form a supramolecular complex containing the nucleic acid complex and a multi-dimensional polymer network. The polymer can be linear or branched. The supramolecular complex can take any suitable form, and preferably, is in the form of particles.

The nucleic acid complex can be delivered via a lipid-mediated delivery system. In some embodiments, the nucleic acid complex can be encapsulated or associated with liposomes. For example, the nucleic acid complex can be condensed with a polycationic condensing agent, suspended in a low-ionic strength aqueous medium and cationic liposomes formed of a cationic vesicle-forming lipid.

As used herein, the term “liposomes” refers to lipid vesicles having an outer lipid shell, typically formed on one or more lipid bilayers, encapsulating an aqueous interior. In some embodiments, the liposomes are cationic liposomes composed of between about 20-80 mole percent of a cationic vesicle-forming lipid, with the remaining neutral vesicle-forming lipids and/or other components. As used herein, “vesicle-forming lipid” refers to any amphipathic lipid having hydrophobic and polar head group moieties and which by itself can form spontaneously into bilayer vesicles in water (e.g. phospholipids). A preferred vesicle-forming lipid is a diacyl-chain lipid, such as a phospholipid, whose acyl chains are typically between about 14-22 carbon atoms in length, and have varying degrees of unsaturation.

A cationic vesicle-forming lipid is a vesicle-forming lipid whose polar head group with a net positive charge, at the operational pH, e.g., pH 4-9. Examples include phospholipids (e.g., phosphatidylethanolamine), glycolipids (e.g., cerebrosides and gangliosides having a cationic polar head-group), cholesterol amine and related cationic sterols (e.g., 1,2-diolelyloxy-3-(trimethylanuno) propane (DOTAP), N-[1-(2,3,-ditetradecyloxy) propyl]-N,N-dimethyl-N-hydroxyethylammonium bromide (DMRIE), N-[1-(2,3,-dioleyloxy) propyl]-N,N-dimethyl-N-hydroxy ethylammonium bromide (DORIE), N-[1-(2,3-dioleyloxy) propyl]-N,N,N-trimethylammonium chloride (DOTMA), 3β [N—(N′,N′-dimethylaminoethane) carbamoyl]cholesterol (DC-Choi), and dimethyldioctadecylammonium (DDAB)).

A neutral vesicle-forming lipid is a vesicle-forming lipid having no net charge or including a small percentage of lipids having a negative charge in the polar head group. Examples of vesicle-forming lipids include phospholipids, such as phosphatidylcholine (PC), phosphatidyl ethanolamine (PE), phosphatidylinositol (PI), and sphingomyelin (SM), and cholesterol, cholesterol derivatives, and other uncharged sterols.

In some embodiments, the delivery systems used herein include, but are not limited to, nanoparticles (NPs), inorganic nanoparticles (e.g. silica NPs, gold NPs, Qdots, superparamagnetic iron oxide NPs, paramagnetic lanthanide ions) and other nanomaterials, nucleic acid lipid particles, polymeric nanoparticles, lipidoid nanoparticles (LNPs), chitosan and inulin nanoparticles, cyclodextrins nanoparticles, carbon nanotubes, liposomes, micellar structures, capsids, polymers (e.g. polyethylenimine, anionic polymers), polymer matrices, hydrogels, dendrimers (e.g. poly-propylenimine (PPI) and poly-amidoamine (PAMAM)), nucleic acid nanostructure, exosomes, and GalNAc-conjugated melittin-like peptides (NAG-MLPs). In some embodiments, the nucleic acid complex can be formulated in buffer solutions such as phosphate buffered saline solutions.

In some embodiments, the nucleic acid complex herein described is delivered via lipidoid nanoparticles (LNPs). LNPs can comprise ionizable LNPs, cationic LNPs, and/or neutral LNPs. Ionizable LNPs are nearly uncharged during circulation but become protonated in a low pH environment, e.g., in the endosomes and lysosomes. Cationic LNPs exhibit a constitutive positive charge in blood circulation and in endosomes or lysosomes. Neutral LNPs are neutral, uncharged during circulation and in endosomes or lysosomes.

The nucleic acid complex herein described can be provided naked or conjugated to a ligand. Naked siRNA refer to a system that contains no delivery system that is associated with the siRNA either covalently or noncovalently. When delivered in naked form, the naked siRNAs can be locally injected to a target site such as specific organs that are relatively closed off and contain few nucleases (e.g. eye).

In some embodiments, the nucleic acid complex herein described can be attached to (e.g. fused or conjugated) a ligand to form ligand-siRNA conjugates that can transport siRNA to desired tissues and cells by specific recognition and interactions between the ligand and the surface receptor of the cells or tissues. Common targeting ligands include carbohydrate, aptamers, antibodies or antibody fragments, peptides (e.g., cell-penetrating peptides, endosomolytic peptides), and small molecules (e.g., N-Acetylgalactosamine (GalNAc)), and others as will be apparent to a skilled artisan.

In some embodiments, the nucleic acid complex is conjugated to an aptamer. The term “aptamers” as used here refers to oligonucleotide or peptide molecules that bind a specific target with high affinity and specificity. In particular, nucleic acid aptamers can comprise, for example, nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. Peptide aptamers are peptides that are designed to specifically bind to and interfere with protein-protein interactions inside cells. In particular, peptide aptamers can be derived, for example, according to a selection strategy that is derived from the yeast two-hybrid (Y2H) system. Aptamers are useful in biotechnological and therapeutic applications as they offer molecular recognition properties that rival that of the antibodies.

In some embodiments, the nucleic acid complex is conjugated to a small molecule. The term “small molecule” as used herein indicates an organic compound that is of synthetic or biological origin and that, although may include monomers and/or primary metabolites, is not a polymer. In some embodiments, small molecules can comprise molecules that are not protein or nucleic acids, which play a biological role that is endogenous (e.g., inhibition or activation of a target) or exogenous (e.g., cell signaling), which are used as a tool in molecular biology, or which are suitable as drugs in medicine. Small molecules can also have no relationship to natural biological molecules. Typically, small molecules have a molar mass lower than 1 kg/mol. Exemplary small molecules include secondary metabolites (e.g., actinomycin-D), certain antiviral drugs (such as amantadine and rimantadine), teratogens and carcinogens (such as phorbol 12-myristate 13-acetate), natural products (such as penicillin, morphine and paclitaxel) and additional molecules identifiable by a skilled artisan. In some embodiments, the nucleic acid complex herein described is conjugated to GalNAc.

Examples of ligands suitable for use in targeting the nucleic acid complex to specific cell types include, but are not limited to, folate capable of binding to folate receptor of epithelial carcinomas and bone marrow stem cells, water soluble vitamins capable of binding to vitamin receptors of various cells, pyridoxyl phosphate capable of binding to CD4 of CD4 lymphocytes, apolipoproteins capable of binding to LDL of liver hepatocytes and vascular endothelial cells, insulin capable of binding to insulin receptor, transferrin capable of binding to transferrin receptor of endothelial cells, galactose capable of binding to asialoglycoprotein receptor of liver hepatocytes, sialyl-Lewisₓ capable of binding to E, P selectin of activated endothelial cells, Mac-1 capable of binding to L selectin of neutrophils and leukocytes, VEGF capable of binding to Flk-1,2 of tumor epithelial cells, basic FGF capable of binding to FGF receptor of tumor epithelial cells, EFG capable of binding to EFG receptor of epithelial cells, VCAM-1 capable of binding to a₄b₁ integrin of vascular endothelial cells, ICAM-1 capable of binding to a_{L}b₂ integrin of vascular endothelial cells, PECAM-1/CD31 capable of binding to aᵥb₃ integrin of vascular endothelial cells and activated platelets, osteopontin capable of binding to aᵥb₁ integrin and abs integrin of endothelial cells and smooth muscle cells in atherosclerotic plaques, RGD sequences capable of binding to aᵥb₃ integrin of tumor endothelial cells and vascular smooth muscle cells, or HIV GP 120/41 or GP120 capable of binding to CD4 of CD4 lymphocytes, and others identifiable to a skilled artisan.

In some embodiments, the delivery of the nucleic acid complex herein described is such that at least 30%, 40%, 50%, 60%, 70%, 80%, 90% or more of the target cells incorporate the nucleic acid complex. In some embodiments, about 0.1-10 nM nucleic acid complex is delivered to the target cells.

### Formulations

Any suitable pharmaceutical formulations can be employed. In some embodiments, the pharmaceutical compositions of the present disclosure may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension: (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the hydrogel composition. The pharmaceutical compositions can comprise one or more pharmaceutically-acceptable carriers.

Formulations useful in the methods disclosed herein include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the RNAi constructs which produces a therapeutic effect. Generally, out of one hundred percent, this amount will range from about 1% to about 99% of active ingredient, preferably from about 5% to about 70%, most preferably from about 10% to about 30%.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a respiration uncoupling agent as an active ingredient. A nucleic acid complex composition may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Actual dosage levels of the active ingredients in the pharmaceutical compositions of the present disclosure may be determined by the methods of the present invention so as to obtain an amount of the active ingredient, which is effective to achieve the desired therapeutic response for a particular subject, composition, and mode of administration, without being toxic to the subject.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more respiration uncoupling agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of hydrogel compositions include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a respiration uncoupling agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Ophthalmic formulations, eye ointments, powders, solutions (e.g. eye drops) and the like, are also contemplated as being within the scope of the present disclosure.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The pharmaceutical compositions herein described comprise therapeutically-effective amount of the nucleic acid complexes.

The phrase “therapeutically-effective amount” as used herein means that amount of nucleic acid complex disclosed herein which is effective for producing some desired therapeutic effect, e.g., cancer treatment, at a reasonable benefit/risk ratio. The therapeutically-effective amount also varies depending on the structure of the constructs, the route of administration utilized, the target sites, and the specific diseases or disorders to be treated as will be understood to a person skilled in the art. For example, if a given clinical treatment is considered effective when there is at least a 20% reduction in a measurable parameter associated with a disease or disorder, a therapeutically-effective amount of the constructs for the treatment of that disease or disorder is the amount necessary to achieve at least a 20% reduction in that measurable parameter.

In some embodiments, the pharmaceutical composition herein described comprises the nucleic acid complex in a suitable dosage sufficient to inhibit expression of the target gene in a subject (e.g. animal or human) being treated. In some embodiments, a suitable dosage of the nucleic acid complex is in the range of 0.001 to 0.25 milligrams per kilogram body weight of the subject per day, or in the range of 0.01 to 20 micrograms per kilogram body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. The pharmaceutical compositions comprising the nucleic acid complex can be administered once daily, twice daily, three times daily or as needed or prescribed by a physician. The pharmaceutical composition herein described can also be provided in dosage units comprising two, three, four, five, six or more sub-doses administered at appropriate intervals throughout the day. The dosage unit can also be compounded for a single dose (e.g. using sustained or controlled release formulation) which can be sustainably released over several days in a controlled manner.

As will be apparent to a skilled person, a suitable dosage unit of the pharmaceutical composition herein described can be estimated from data obtained from cell culture assays and further determined from data obtained in animal studies. For example, toxicity and therapeutic efficacy of the pharmaceutical compositions described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred. Suitable dosages of the compositions in combination with particular delivery systems can be selected in order to minimize toxicity, such as to minimize potential damage to untargeted cells and to reduce side effects.

### Administration

As will be apparent to a skilled artisan, the nucleic acid complexes herein described and compositions thereof can be administrated to a subject using any suitable administration routes. The nucleic acid complexes and compositions thereof can be administered to a target site locally or systematically.

The wording “local administration” or “topic administration” as used herein indicates any route of administration by which a composition is brought in contact with the body of the individual, so that the resulting composition location in the body is topic (limited to a specific tissue, organ or other body part where the imaging is desired). Exemplary local administration routes include injection into a particular tissue by a needle, gavage into the gastrointestinal tract, and spreading a solution containing hydrogel composition on a skin surface.

The wording “systemic administration” as used herein indicates any route of administration by which a nucleic acid complex composition is brought in contact with the body of the individual, so that the resulting composition location in the body is systemic (i.e. non limited to a specific tissue, organ or other body part where the imaging is desired). Systemic administration includes enteral and parenteral administration. Enteral administration is a systemic route of administration where the substance is given via the digestive tract, and includes but is not limited to oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion.

In some embodiments, the methods of administration can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous (IV) injection, subcutaneous (SC) injection, intranodal injection, intratumoral injection, intraperitoneal injection, and/or intradermal injection, or any combination thereof. The administration can also be site-specific injection (e.g. in the eye or the cerebral spinal fluid).

In some embodiments, the administration can be Ex vivo transduction, cell injection, subcutaneous injection, intravenous injection, intrathecal delivery, intracerebroventricular injection, intradermal injection, intravitreal delivery, intratumoral delivery, or topical application (e.g. topical eye drop).

The methods of administration depends on the target site, the type of cells/tissues to be targeted at, and how the constructs are formulated. In some embodiments, lipid formulations can be administered to animals such as by intravenous, intramuscular, or intraperitoneal injection, or orally or by inhalation or other methods as known in the art.

In some embodiments, the administration can be SC injection into the adipose tissue below the epidermis and dermis. In some embodiments, SC administration can be associated with ligand-conjugated nucleic acid complex herein described. In some embodiments, SC administration can render a slower release rate of the drugs into the systemic circulation and an entering into the lymphatic system, giving more time for recycling of cellular receptors that mediate uptake. In some embodiments, SC administration can be faster and easier to administer, reducing treatment burden.

In some embodiments, the administration can be any administration route allowing the penetration of drugs through the blood brain barrier. The blood brain barrier is very selective and serves to prevent potentially harmful substances in the blood from entering into the CNS. In some embodiments, the route of administration can be direct brain injection, transmembrane diffusion, or intraventricular infusion of therapeutic substances directly into the cerebrospinal fluid. In some embodiments, the administration can be intrastriatal injection, intrathecal injection, intracerebral injection, intracranial injection, intraparenchymal injection, intranasal delivery or intracerebroventricular injection. In some embodiments, the administration can be intracerebroventricular injection into the CNS to bypass the blood-brain barrier and other mechanisms that limit drug distribution to the brain, allowing a higher drug concentration to enter the central compartment.

IV administration can, for example, be associated with nanoparticle and lipid nanoparticle formulated nucleic acid complex herein described. In some embodiments, IV administration can avoid first-pass metabolism in the liver and affords quick access to target tissue through the systemic circulation.

### Target Sites

The compositions herein described can be administered to any suitable target site. In some embodiments, the target site is the central nervous system (e.g., brain and spinal cord), peripheral nervous system (e.g., nerves that branch off from the spinal cord) and connective tissues/organs involving in the function and pathways between the central and peripheral nervous systems (e.g., dorsal root and ventral root). The target site can include the brain, spinal cord, cranial nerves, peripheral nerves, nerve roots, autonomic nervous system, neuromuscular junction and muscles. In some embodiments, the target site is the central nervous system.

The target site can comprise a site of disease or disorder or can be proximate to a site of a disease or disorder. The location of the one or more sites of a disease or disorder can be predetermined. The location of the one or more sites of a disease or disorder can be determined during the method (e.g., by an imaging-based method such as ultrasound or MRI). The target site can comprise a tissue, such as, for example, grey matter, white matter, ganglion, nerves, endoneurium, perineurium, epineurium.

In some embodiments, target sites where the nucleic acid complex or compositions thereof can be administered can vary in different embodiments depending on the mode of administration utilized and the types of diseases or disordered to be treated.

The term “individual” or “subject” or “patient” as used herein in the context of imaging includes an animal and in particular higher animals and in particular vertebrates such as mammals and more particularly human beings.

In some embodiments, the ratio of the concentration of the nucleic acid complex at the subject's target site (e.g., CNS) to the concentration of the nucleic acid complex outside the target site (e.g. in subject's blood circulation, serum, or plasma) can vary. In some embodiments, the ratio of the concentration of the nucleic acid complex at the subject's target site (e.g., CNS) to the concentration of the nucleic acid complex outside the target site (e.g. in subject's blood circulation, serum, or plasma) can be, or be about, be at least, be at least about, be at most, or be at most about, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values.

The target site can comprise target cells. The target cells can include nerve cells and glial cells, including pyramidal cells, purkinje cells, granule cells, spindle neurons, nedium spiny neurons, interneurons, astrocyte, ependymal cells, microglia, oligodendrocyte, and oligodendrocyte progenitor cells. The target cells can also include dorsal root ganglion, ventral root ganglion, and automonic ganglion.

In some embodiments, the administration of the nucleic acid complex and/or compositions herein described to a target site of the subject results in at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or a number or a range between any two of these values, reduction in the target nucleic acid expression in the target cells. In some embodiments, the ratio of reduction in the target nucleic acid in the target cells to non-target cell after administration of the nucleic acid complex and/or compositions can be at least about 2:1. In some embodiments, the ratio can be, or be about, or be at least, or be at least about, or be at most, or be at most about, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values.

### Methods of Treating a Neurological Disease or Disorder

Also provided herein is a method of treating a disease or a disorder using the nucleic acid complex or a composition thereof herein described. The method can comprise administering the nucleic acid complex described herein to a subject in need thereof. Upon detection of an input nucleic acid strand (e.g., disease biomarker), the input nucleic acid strand can bind to the overhang of the sensor nucleic acid strand to cause displacement of the sensor nucleic acid strand from the core nucleic acid strand to release the sequence complementary to a target RNA, thereby reducing the activity of the target RNA or protein expression from the target RNA in the subject to treat the disease or condition. In some embodiments, the disease or disorder is a neurological disease or disorder, a neurodegenerative disease or disorder, and/or a disease or disorder of the central nervous system (CNS).

The term “condition” as used herein indicates a physical status of the body of an individual (as a whole or as one or more of its parts), that does not conform to a standard physical status associated with a state of complete physical, mental and social well-being for the individual. Conditions herein described include but are not limited disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms.

Various diseases and disorders can be treated with the nucleic acid complex compositions provided herein. In some embodiments, the disease or disorder can be a neurological disease or disorder. Neurological diseases or disorders are diseases or disorders of the central and peripheral nervous system including the brain, spinal cord, cranial nerves, peripheral nerves, nerve roots, autonomic nervous system, neuromuscular junction, and muscles. Neurological disorders can include epilepsy, Alzheimer's disease and other dementias, cerebrovascular diseases including stroke, migraine and other headache disorders, multiple sclerosis, Parkinson's disease, neuroinfections, brain tumors, and traumatic disorders of the nervous system due to head trauma.

Exemplary neurological diseases or disorders include, but are not limited to, Absence of the Septum Pellucidum, Acid Lipase Disease, Acute Disseminated Encephalomyelitis, Adrenoleukodystrophy, Agenesis of the Corpus Callosum, Agnosia, Aicardi Goutieres Syndrome Disorder, Aicardi Syndrome, Alexander Disease, Alpers Disease, ALS Amyotrophic Lateral Sclerosis, Alternating Hemiplegia, Alzheimer's Disease, Amyotrophic Lateral Sclerosis ALS, Anencephaly, Angelman Syndrome, Antiphospholipid Syndrome, Aphasia, Apraxia, Arachnoid Cysts, Arachnoiditis, Arteriovenous Malformation, Asperger Syndrome, Ataxia Telangiectasia, Ataxias and Cerebellar or Spinocerebellar Degeneration, Atrial Fibrillation and Stroke, Attention Deficit Hyperactivity Disorder, Autism, Autism Spectrum Disorder, Back Pain, Barth Syndrome, Batten Disease, Behcet's Disease, Bell's Palsy, Benign Essential Blepharospasm, Binswanger's Disease, Brachial Plexus Injuries, Brain and Spinal Tumors, Brown Sequard Syndrome, CADASIL, Canavan Disease, Carpal Tunnel Syndrome, Central Cord Syndrome, Central Pain Syndrome, Central Pontine Myelinolysis, Cephalic Disorders, Cerebellar Degeneration, Cerebellar Hypoplasia, Cerebral Aneurysms, Cerebral Arteriosclerosis, Cerebral Atrophy, Cerebral Cavernous Malformation, Cerebral Hypoxia, Cerebral Palsy, Cerebro Oculo Facio Skeletal Syndrome COFS, Charcot Marie Tooth Disease, Chiari Malformation, Chorea, Chronic Inflammatory Demyelinating Polyneuropathy CIDP, Chronic Pain, Coffin Lowry Syndrome, Colpocephaly, Coma, Complex Regional Pain Syndrome, Congenital Myasthenia, Congenital Myopathy, Corticobasal Degeneration, Craniosynostosis, Creutzfeldt Jakob Disease, Cushing's Syndrome, Dandy Walker Syndrome, Deep Brain Stimulation for Parkinson's Disease, Dementia, Dementia With Lewy Bodies, Dermatomyositis, Developmental Dyspraxia, Diabetic Neuropathy, Dravet Syndrome, Dysautonomia, Dysgraphia, Dyslexia, Dyssynergia Cerebellaris Myoclonica, Dystonias, Empty Sella Syndrome, Encephalitis Lethargica, Encephaloceles, Encephalopathy, Epilepsy, Erb Duchenne and Dejerine Klumpke Palsies, Essential Tremor, Fabry Disease, Fahr s Syndrome, Familial Periodic Paralyses, Farber's Disease, Febrile Seizures, Fibromuscular Dysplasia, Foot Drop, Friedreich's Ataxia, Frontotemporal Dementia, Gaucher Disease, Generalized Gangliosidoses, Gerstmann's Syndrome, Gerstmann Straussler Scheinker Disease, Giant Axonal Neuropathy, Glossopharyngeal Neuralgia, Guillain Barre Syndrome, Headache, Hemicrania Continua, Hemifacial Spasm, Hereditary Neuropathies, Hereditary Spastic Paraplegia, Herpes Zoster Oticus, Holmes Adie syndrome, Holoprosencephaly, Huntington's Disease, Hydranencephaly, Hydrocephalus, Hydromyelia, Hypersomnia, Hypertonia, Hypotonia, Inclusion Body Myositis, Incontinentia Pigmenti, Infantile Neuroaxonal Dystrophy, Infantile Refsum Disease, Infantile Spasms, Inflammatory Myopathies, Iniencephaly, Isaac's Syndrome, Kearns Sayre Syndrome, Kennedy's Disease, Kleine Levin Syndrome, Klippel Feil Syndrome, Klippel Trenaunay Syndrome KTS, Krabbe Disease, Lambert Eaton Myasthenic Syndrome, Landau Kleffner Syndrome, Learning Disabilities, Leigh's Disease, Lennox Gastaut Syndrome, Lesch Nyhan Syndrome, Leukodystrophy, Lipid Storage Diseases, Lipoid Proteinosis, Lissencephaly, Locked In Syndrome, Machado Joseph Disease, Megalencephaly, Melkersson Rosenthal Syndrome, Meningitis and Encephalitis, Menkes Disease, Meralgia Paresthetica, Metachromatic Leukodystrophy, Microcephaly, Migraine, Miller Fisher Syndrome, Mitochondrial Myopathies, Mitochondrial Myopathy, Moebius Syndrome, Monomelic Amyotrophy, Motor Neuron Diseases, Moyamoya Disease, Mucolipidoses, Mucopolysaccharidoses, Multi Infarct Dementia, Multifocal Motor Neuropathy, Multiple Sclerosis, Multiple System Atrophy, Multiple System Atrophy with Orthostatic Hypotension, Muscular Dystrophy, Myasthenia Gravis, Myoclonus, Myopathy, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Sequelae Of Lupus, Neuromyelitis Optica, Neuronal Migration Disorders, Neurosarcoidosis, Neurosyphilis, Neurotoxicity, Niemann Pick Disease, Normal Pressure Hydrocephalus, Occipital Neuralgia, Ohtahara Syndrome, Olivopontocerebellar Atrophy, Opsoclonus Myoclonus, Orthostatic Hypotension, Paraneoplastic Syndromes, Paresthesia, Parkinson's Disease, Paroxysmal Choreoathetosis, Paroxysmal Hemicrania, Parry Romberg, Pelizaeus Merzbacher Disease, Peripheral Neuropathy, Periventricular Leukomalacia, Pervasive Developmental Disorders, Pinched Nerve, Piriformis Syndrome, Pituitary Tumors, Polymyositis, Pompe Disease, Porencephaly, Post Polio Syndrome, Postural Tachycardia Syndrome, Primary Lateral Sclerosis, Progressive Multifocal Leukoencephalopathy, Progressive Supranuclear Palsy, Prosopagnosia, Pseudotumor Cerebri, Psychogenic Movement, Rasmussen's Encephalitis, Refsum Disease, Repetitive Motion Disorders, Restless Legs Syndrome, Rett Syndrome, Reye's Syndrome, Sandhoff Disease, Schilder's Disease, Schizencephaly, Septo Optic Dysplasia, Shaken Baby Syndrome, Shingles, Sjögren's Syndrome, Sleep Apnea, Sotos Syndrome, Spasticity, Spina Bifida, Spinal Cord Infarction, Spinal Cord Injury, Spinal Muscular Atrophy, Stiff Person Syndrome, Striatonigral Degeneration, Stroke, Sturge Weber Syndrome, Subacute Sclerosing Panencephalitis, SUNCT Headache, Swallowing Disorders, Sydenham Chorea, Syncope, Syringomyelia, Tabes Dorsalis, Tardive Dyskinesia, Tarlov Cysts, Tay Sachs Disease, Tethered Spinal Cord Syndrome, Thoracic Outlet Syndrome, Thyrotoxic Myopathy, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vasculitis Syndromes of the Central and Peripheral Nervous Systems, Von Hippel Lindau Disease VHL, Wallenberg s Syndrome, Wernicke Korsakoff Syndrome, Whiplash, Whipple's Disease, Williams Syndrome, Wilson Disease, and Zellweger Syndrome.

In some embodiments, a disease or a disorder is a neurodegenerative disease or disorder. Neurodegenerative diseases or disorders are a heterogeneous group of disorders that are characterized by the progressive degeneration of the structure and function of the central nervous system or peripheral nervous system. In some embodiments, neurodegenerative diseases are diseases marked by continuous and progressive deterioration of the function of neural cells which are not caused by any underlying trauma or infection. Exemplary neurodegenerative diseases or disorders include, but are not limited to, Alzheimer's disease (AD), Huntington's disease (HD), Parkinson's disease (PD), amyotrophic lateral sclerosis (ALS), and prion diseases.

In some embodiments, a disease or a disorder is a disease or condition of the central nervous system (CNS). Exemplary disease or a condition of the CNS include, but are not limited to, Adrenoleukodystrophy, Alzheimer disease, Amyotrophic lateral sclerosis, Angelman syndrome, Ataxia telangiectasia, Charcot-Marie-Tooth syndrome, Cockayne syndrome, Deafness, Duchenne muscular dystrophy, Epilepsy, Essential tremor, Fragile X syndrome, Friedreich's ataxia, Gaucher disease, Huntington disease, Lesch-Nyhan syndrome, Maple syrup urine disease, Menkes syndrome, Myotonic dystrophy, Narcolepsy, Neurofibromatosis, Niemann-Pick disease, Parkinson disease, Phenylketonuria, Prader-Willi syndrome, Refsum disease, Rett syndrome, Spinal muscular atrophy, Spinocerebellar ataxia, Tangier disease, Tay-Sachs disease, Tuberous sclerosis, Von Hippel-Lindau syndrome, Williams syndrome, Wilson's disease, and Zellweger syndrome.

In some embodiments, the CNS disease is a movement disorder, a memory disorder, addiction, attention deficit/hyperactivity disorder (ADHD), autism, bipolar disorder, depression, encephalitis, epilepsy/seizure, migraine, multiple sclerosis, a neurodegenerative disorder, a psychiatric disease, a neuroinflammatory disease, Alzheimer's disease, Huntington's disease, Parkinson's disease, Tourette syndrome, dystonia, or a combination thereof. In some embodiments, the disease is a neuroinflammatory disease. For example, the neuroinflammatory disease is Parkinson's disease, Alzheimer's disease, multiple sclerosis, or a combination thereof. In some embodiments, the CNS disease is Huntington's disease.

In some embodiments, the disease or disorder can be a CNS disease or condition. The nucleic acid complex herein described or a composition thereof can be administered to the cells, tissues and/or organs of the CNS using any suitable administration route herein described. For example, the nucleic acid complex or a composition thereof can be administered to the cells, tissues and/or organs of the CNS of a subject via intrathecal injection, intracerebroventricular injection, or intracerebral injection to penetrate the blood-brain barrier. In some embodiments, the cell(s), tissue(s), and/or organ(s) of the CNS comprises damaged or inflamed cell(s), tissue(s), or organ(s). In some embodiments, the cells(s), tissue(s), and/or organ(s) of the CNS comprise the brain, the white matter, the gray matter, the brainstem, the cerebellum, the diencephalon, the cerebrum, the spinal cord, the cranial nerve, cell(s) of any of the preceding, tissue(s) of any of the preceding, or a combination thereof.

In some embodiments, the method described herein comprises administering a nucleic acid complex herein described to a subject in need thereof, allowing the nucleic acid complex to be distributed into one or more regions of the nervous system in which the input nucleic acid strand binds to the overhang of the third nucleic acid strand to cause displacement of the third nucleic acid strand from the first nucleic acid strand to release the sequence complementary to a target RNA, thereby reducing the activity of the target RNA or protein expression from the target RNA in the one or more regions of the nervous system of the subject to treat the neurological disease or disorder. In some embodiments, the one or more regions of the nervous systems comprise a central nervous system, a peripheral nervous system, or both.

In some embodiments, the central nervous system comprises the brain, the white matter, the gray matter, the brainstem, the cerebellum, the diencephalon, the cerebrum, the spinal cord, the cranial nerve, or a combination thereof. In some embodiments, the one or more regions of the central nervous system comprise spinal cord, cerebrum (e.g., frontal lobe, parietal lobe, occipital lobe, temporal lobe, left hemisphere, and right hemisphere), cerebral cortex (e.g., prefrontal cortex, sensory cortex, visual cortex, auditory cortex, motor cortex), basal ganglia (e.g., striatum), thalamus, subthalamus, epithalamus, hypothalamus, amygdala, hippocampus (e.g., ventral hippocampus, dorsal hippocampus, and intermediate hippocampus), cerebellum, brain stem (e.g., midbrain, pons, medulla oblongata), left hemisphere, right hemisphere, corpus callosum, or a combination thereof. In some embodiments, the one or more regions of the CNS comprises cerebral cortex, subicular cortex, hippocampus, corpus callosum, fornix, lateral ventricle, stria terminalis, caudate putamen, internal capsule, piriform cortex, globus pallidus, optic tract, amygdala, anterior commissure, ventral striatum, lateral olfactory tract, cerebellum, pons, medulla, middle cerebellar peduncle, or a combination thereof. In some embodiments, the one or more regions of the CNS comprises right cortex, prefrontal cortex, sensory cortex, visual cortex, striatum, dorsal hippocampus, ventral hippocampus, thalamus, cerebellum, midbrain, left hemisphere, right hemisphere, or a combination thereof. In some embodiments, the one or more regions of the nervous system comprises connective tissues/organs involving in the function and pathways between the central and peripheral nervous systems. In some embodiments, the one or more regions of the nervous system comprises dorsal root ganglion that carries sensory neural signals to the CNS from the PNS.

The target RNA can comprise any gene described herein or known in the art whose expression or activity is associated with a neurological disease or disorder (e.g., a neurodegenerative disease). For example, in some embodiments, the neurological disease is Huntington's disease and the target RNA comprises a HTT gene.

The sensor nucleic acid strand of the nucleic acid complex can be designed to detect any biomarker described herein or known in the pertinent art which is related to a neurological disease or disorder such as a disease or disorder of the central nervous system. The biomarker can be a cell type or cell-state specific or selective mRNA such as mRNAs specific for cells of the central nervous system (e.g., GFAP mRNA). The biomarker can also be a universal mRNA that is not cell type specific or selective (e.g., mir-23a-3p, mir-21-5p, mir-29c-3p, 5.8s ribosomal RNA).

In some embodiments, the nucleic acid complex is administered to the subject in need thereof at a concentration about 0.001-10 nM. For example, the nucleic acid complex can be provided at a concentration of, about, at most, or at most about, 0.001 nM, 0.002 mM, 0.004 mM, 0.006 mM, 0.008 mM, 0.01 nM, 0.02 nM, 0.03 nM, 0.04 nM, 0.05 nM, 0.06 nM, 0.07 nM, 0.08 nM, 0.09 nM, 0.1 nM, 0.2 nM, 0.3 nM, 0.4 nM, 0.5 nM, 0.6 nM, 0.7 nM, 0.8 nM, 0.9 nM, 1.0 nM, 1.5 nM, 2.0 nM, 2.5 nM, 3.0 nM, 3.5 nM, 4.0 nM, 4.5 nM, 5.0 nM, 5.5 nM, 6.0 nM, 6.5 nM, 7.0 nM, 7.5 nM, 8.0 nM, 8.5 nM, 9.0 nM, 9.5 nM, 10 nM, or a number or a range between any two of these values. In some embodiments, the nucleic acid complex is provided at a concentration about 0.004-1.0 nM.

In some embodiments, the nucleic acid complex is administered to the subject in need thereof at a dosage about 1-100 mg/kg body weight of the subject, preferably 10-50 mg/kg body weight of the subject. Alternatively dosages may be based and calculated based upon the subject being treated, the severity and responsiveness of the condition to be treated, the manner of administration, and the judgement of the prescribing physician, as understood by those of skill in the art. In some embodiments, the subject can be administered with the nucleic acid complex one, two, three, four or more times for the treatment. In some embodiments, at most one, two, three or four administrations are needed to achieve a desired treatment outcome. In some embodiments, only one administration is needed. Two administrations of the nucleic acid complex can be separated by a suitable time period. The suitable time period between two administrations can be the same as or different from the suitable time period between another two administrations. In some embodiments, the time period between two administrations can be about, at least or at least about 1 month, 2 months, 3 months, 4 months, 5 months, 6 months, 7 months, 8 months, 9 months, 10 months, 11 months, 12 months or longer. In some embodiments, the time period between any two administrations can be at least 6 months.

In some embodiments, the administration of the nucleic acid complex to a subject in need thereof results in reduction or loss of expression of the target nucleic acid in the target cells. In some embodiments, the reduction of the target nucleic acid after the administration of the nucleic acid complex herein described is about, at least, or at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two values, with respect to the level of target nucleic acid prior to the administration. In some embodiments, the reduction occurs in one or more of the regions selected from the group consisting of: right cortex, prefrontal cortex, sensory cortex, visual cortex, striatum, dorsal hippocampus, ventral hippocampus, thalamus, cerebellum, midbrain, left hemisphere, right hemisphere, or a combination thereof.

In some embodiments, following the administration of the nucleic acid complex, the expression of the target nucleic acid is reduced by about, at least, or at least about 50%, 60%, 70%, 80%, 90%, 95%, 99%, or a number or a range between any two of these values, in right cortex, striatum, hippocampus, thalamus, and/or cerebellum. In some embodiments, following the administration of the nucleic acid complex, the expression of the target nucleic acid is reduced by about, at least, or at least about 50%, 60%, 70%, 80%, 90%, 95%, 99%, or a number or a range between any two of these values, in prefrontal cortex, sensory cortex, visual cortex, striatum, dorsal hippocampus, ventral hippocampus, thalamus, cerebellum, midbrain, left hemisphere, and/or right hemisphere.

The experimental results have demonstrated that the nucleic acid complex is specific and potent while achieving safety and tolerability in the subject being treated. For example, the administration of the nucleic acid complex does not result in a significant increase or decrease (e.g., plus or minus 5% of a base value or the difference is not statistically significant) in the body weight, inflammatory markers, blood chemistry, and/or liver, kidney pancreas enzymes in the subject with respect to the levels prior to the administration. In some embodiments, the administration of the nucleic acid complex does not induce unintended inflammatory responses. For example, in some embodiments, the administration the nucleic acid complex does not result an elevated glial fibrillary acidic protein (GFAP) and/or ionized calcium-binding adapter molecule 1 (IBA-1) level of the subject with respect to the levels prior to the administration.

### Kits

Also provided herein include kits for preventing or treating a neurological disease. The kits can comprise one or more compositions described herein, in suitable packaging such as in a container, pack, or dispenser, and may further comprise written material that can include instructions for use, discussion of clinical studies, listing of side effects, and the like. For example, a kit can comprise instructions for using the components of the kit to practice the methods described herein such as to treat or prevent a neurological disease or disorder (e.g., a disease or disorder of the CNS). Such kits can also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information can be based on the results of various studies, for example, studies using experimental animals involving in vivo models and studies based on human clinical trials. A kit can comprise one or more unit doses described herein. The compositions can be in the form of kits of parts. In a kit of parts, one or more components of the compositions disclosed herein are provided independent of one another (e.g., constructs, excipients, and/or diluents are provided as separate compositions) and are then employed (e.g., by a user) to generate the compositions.

### EXAMPLES

Some aspects of the embodiments discussed above are disclosed in further detail in the following examples, which are not in any way intended to limit the scope of the present disclosure.

### Example 1

### Investigation of CASi Stability in Serum and Cellular Uptake

In this example, the ability of CASi against degradation by serum components was studied by agarose gel electrophoresis. CASi was incubated with 33% fetal bovine serum (FBS). Aliquots were taken at 24 h, 48 h, 72 h, 96 h, 120 h, 144 h and 168 h. FIG. 7 demonstrates the stability of CASi complexes to serum degradation over time. No degradation was observed after 7 days in the FBS.

The CASi complex was also evaluated for siRNA uptake efficiency. 50 nM Cy3 labeled CASi (red) bearing either no ligand, a palmitic acid ligand (Ligand 1) or a cholesterol ligand (Ligand 2) at the 3′ terminus of the sensor strand was added to cell media and incubated with Hela cells. After 24 hr posttreatment, cells were subjected to DAPI staining (blue). Localization of the siRNA complex was monitored by microscopy. FIG. 8 shows the localization of siRNA. The results demonstrate that cellular uptake of CASi from media is readily detectable by microscopy after 24 hours.

### Example 2

### CNS Uptake and Distribution of CASi

This example evaluates how extensive the CASi complex can distribute throughout the central nervous system (CNS) via intracerebroventricular (ICV) administration.

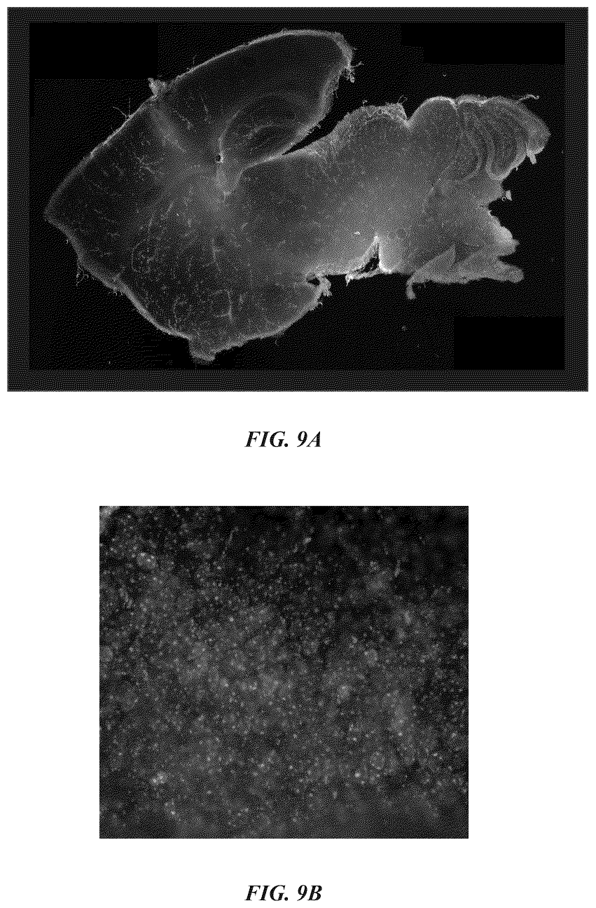

Intracerebroventricular injections of 1 nM or 3 nM Cy3-labeled CASi was performed on mice. Mice were euthanized at 24 or 48 hours and brain tissues collected for fluorescence microscopy. FIG. 9A shows the distribution of CASi in the CNS 24 hours after injection. The results demonstrate a broad distribution of the CASi via imaging for fluorescently tagged CASi. The Cy3 signal was found to have antisense oligonucleotide (ASO)-like association with capillaries. To monitor CASi uptake, select regions of collected brain tissues treated with 3 nM Cy3-labeled CASi were imaged at 20× magnification. FIG. 9B demonstrates the CNS cellular uptake of CASi after 24 hours. The co-localization of the punctate features with cells suggests a productive CASi uptake.

### Example 3

### Evaluation of CASi Tolerance In Vivo and in the CNS

This example evaluates the tolerance of CASi complex in vivo and in the CNS.

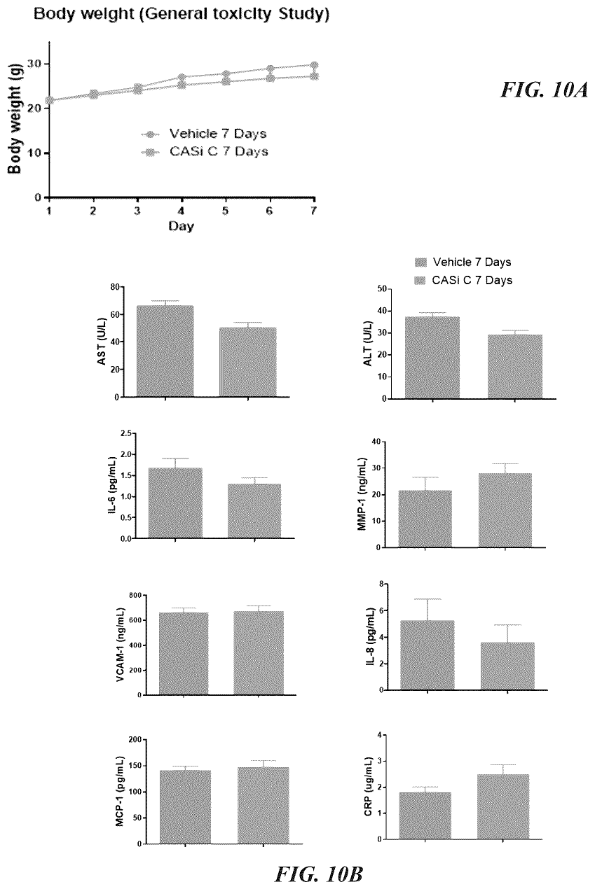

CASi C was administered to healthy mice at a dose of 20 mg/kg via subcutaneous injection route. In CASi C, the sensor comprises a mir-23 sensor 8 nt with cholesterol at 3′ terminus (instead of palmitic acid), the passenger strand is an HTT passenger strand, and the core strand is a mir23-HTT core strand. Body weight and blood chemistry including inflammatory markers, liver, kidney, and pancreas enzymes were monitored. FIG. 10A is a diagram illustrating the body weights of the group administered with CASi C in comparison to a control group administrated with saline. No significant difference in the body weight was observed between the two groups. FIG. 10B provides diagrams showing aspartate aminotransferase (AST) (U/L), alanine aminotransferase (ALT) (U/L), IL-6 (pg/mL), matrix metalloproteinase-1 (MMP-1) (ng/mL), vascular cell adhesion molecule 1 (VCAM-1) (ng/ml), IL-8 (pg/mL), monocyte chemoattractant protein-1 (MCP-1) (pg/mL), C-reactive protein (CRP) (μg/mL) of the group administered with CASi C in comparison to a control group. No significant differences were observed between the two groups.

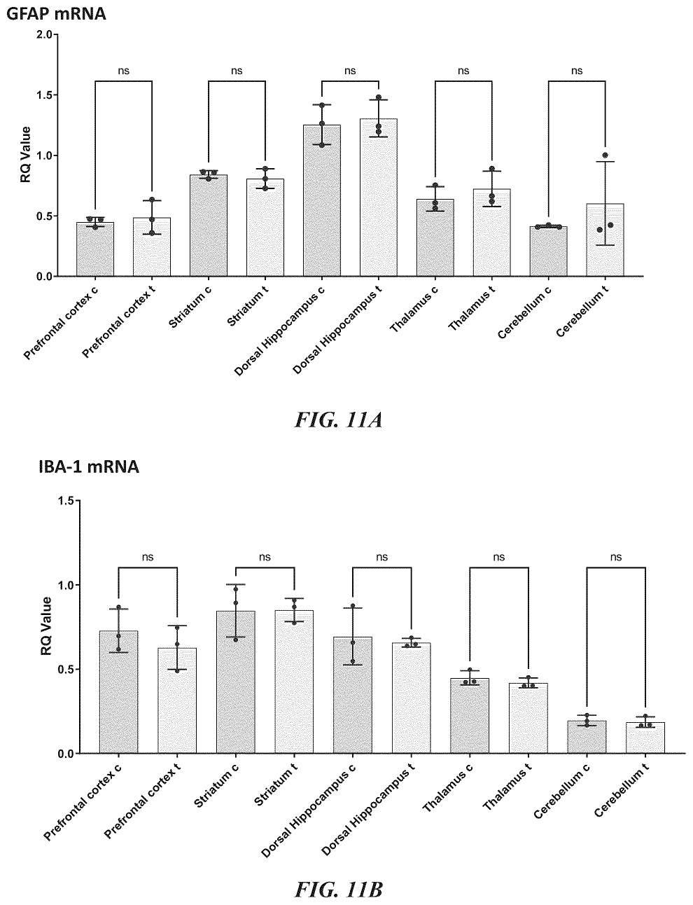

CASi was also administered to mice via ICV injection at 425 μg to test for CNS inflammation by measuring astrocyte (GFAP) and microglia (IBA-1) activation. FIGS. 11A-B are graphs showing the GFAP mRNA (FIG. 11A) and IBA-1 mRNA (FIG. 11B) levels in various brain regions of CASi treated animals (t) in comparison to saline treated animals (c). No significant differences in GFAP and IBA-1 levels were found from saline injected animals 14 days after the CASi treatment.

### Example 4

### RNAi Activity of CASi Constructs

This example demonstrates the RNAi activity of CASi constructs with various CASi constructs and siRNA domains thereof.

The RNAi activities of the CASi siRNA segments were measured using dual luciferase assays. CASi constructs or siRNA domains were co-transfected into HCT 116 cells with dual luciferase vectors carrying the Huntingtin gene siRNA target sequence, using lipofectamine 2000. After 48 hours, cells were lysed and assayed for knockdown of the target gene by comparing the luminescence value of Renilla luciferase that carries the target sequence to Firefly luciferase that was used as a reference control.

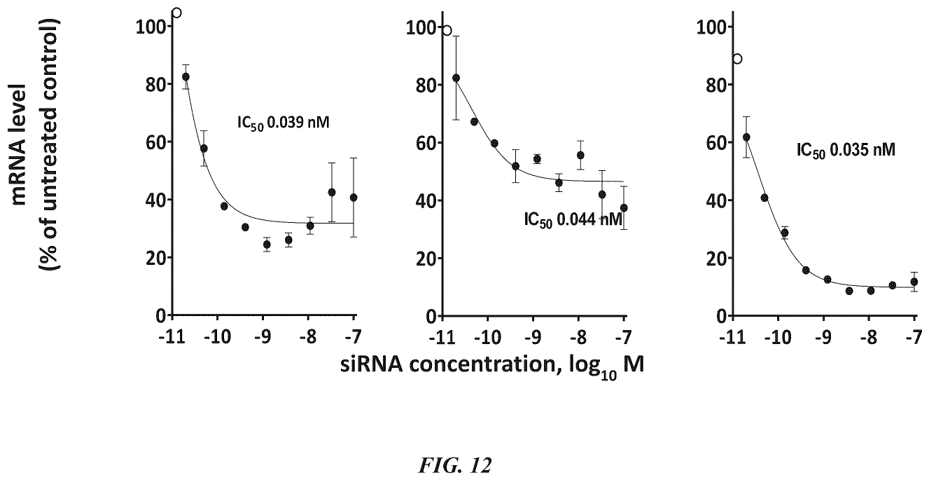

FIG. 12 are graphs showing the RNAi activity of exemplary siRNA candidates. The siRNA candidates evaluated herein demonstrate competitive potency and specificity with leading therapeutic siRNA platforms. The results also demonstrate that the siRNA candidates can be combined with any optimized cell-selective or non-selective CASi sensor (e.g., universal sensors) to form lead compounds.

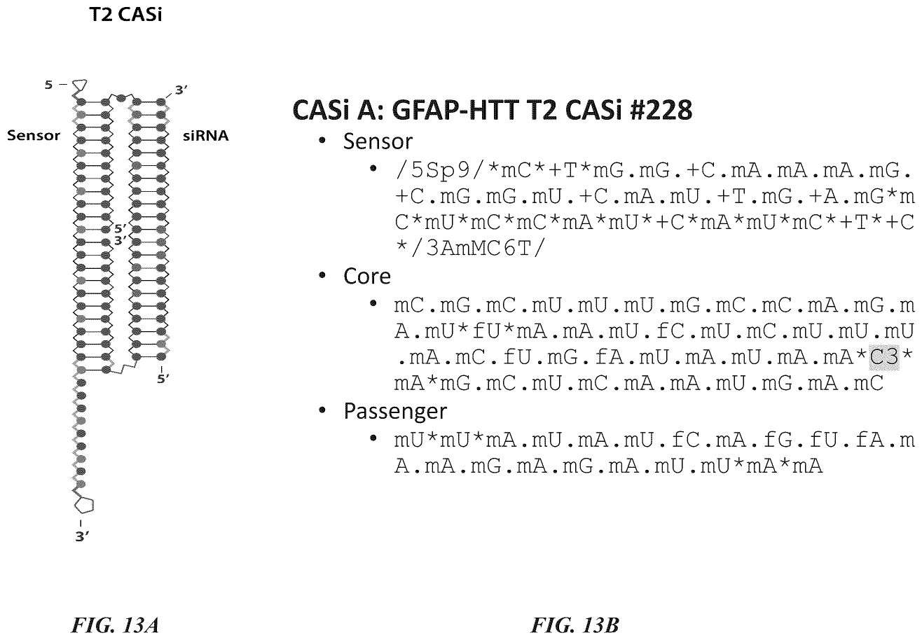

FIG. 13A illustrates a schematic representation of a non-limiting exemplary nucleic acid complex construct T2 CASi. FIG. 13B shows sequence diagrams of CASi A construct (also referred to as “GFAP-HTT T2 CASi #228) comprising a sensor strand (SEQ ID NO: 1), a core strand (SEQ ID NO: 2) and a passenger strand (SEQ ID NO: 3). The CASi A construct is designed to detect both primate and rodent glial fibrillary acidic protein mRNA (GFAP mRNA). The CASi A construct is designed to target the primate and rodent Huntingtin gene mRNA for RNAi silencing.

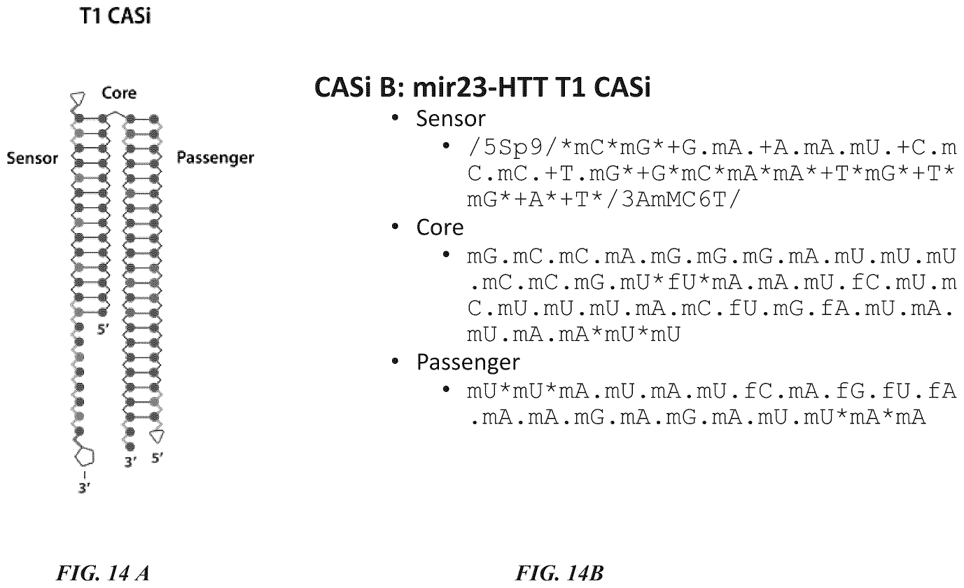

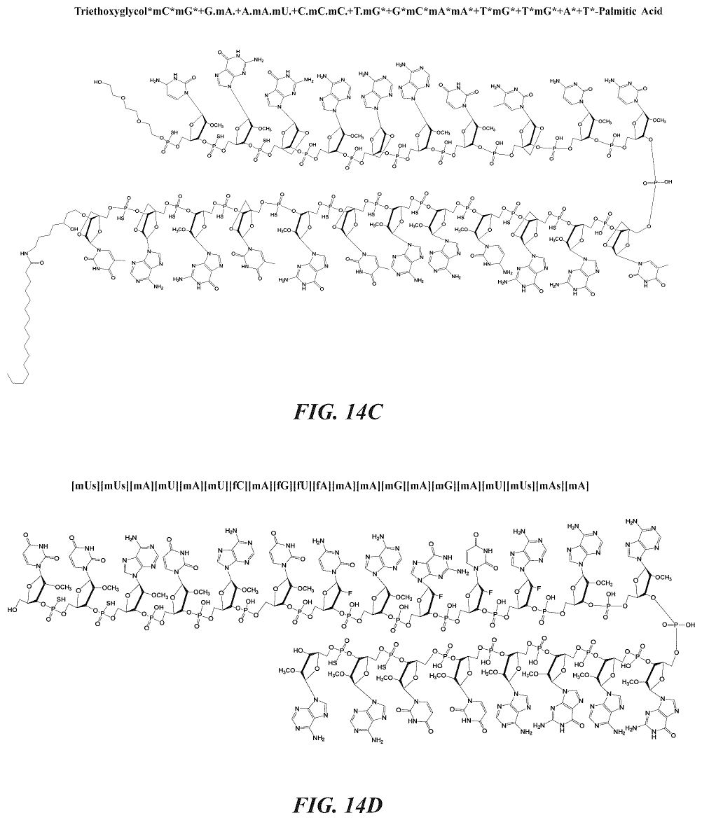

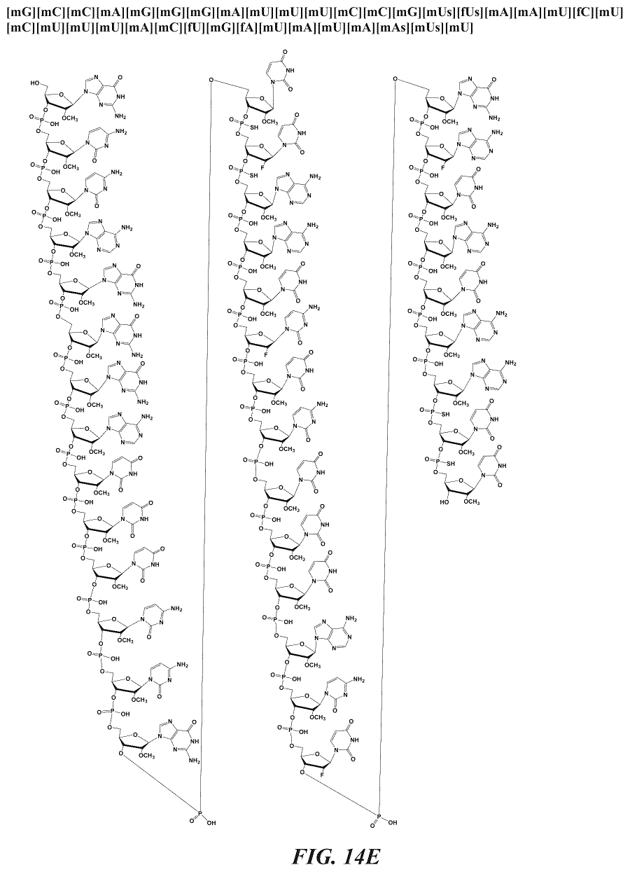

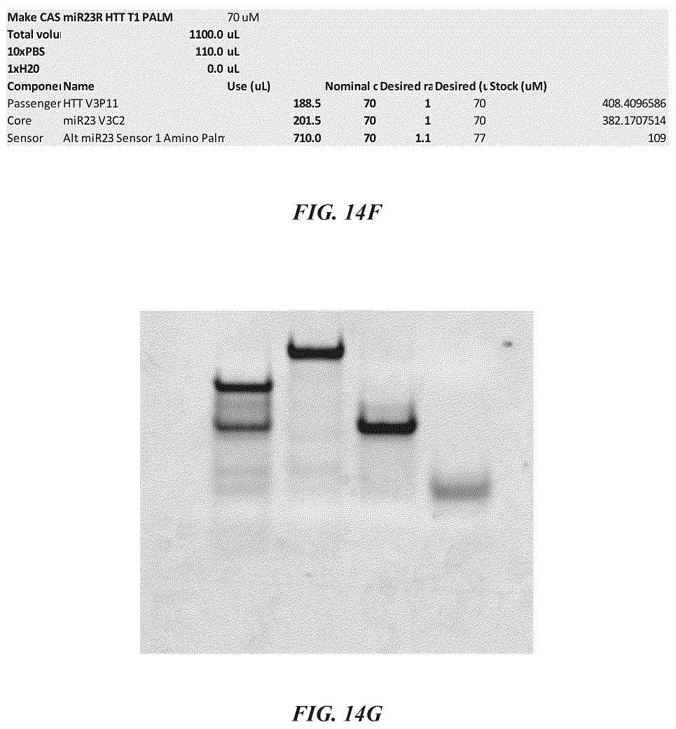

FIG. 14A illustrates a schematic representation of a non-limiting exemplary nucleic acid complex construct T1 CASi. FIG. 14B shows sequence diagrams of CASi B construct (also referred to as “mir23-HTT T1 CASi”) comprising a sensor strand (SEQ ID NO: 4) (with a standard 8 nucleotide toehold), a core strand (SEQ ID NO: 5) and a passenger strand (SEQ ID NO: 3). The CASi B construct is designed to detect a universal mir-23a-3p microRNA. The CASi B construct is designed to target the primate and rodent Huntingtin gene mRNA for RNAi silencing.

Table 1 below provides sequence diagrams of universal CASi constructs and strands. The CASi constructs provided herein in Table 1 comprise a universal sensor that can detect a universal mRNA, i.e., non-cell type selective mRNA. All constructs listed below target the Huntingtin gene mRNA for RNAi silencing and detect the microRNA or non-coding RNAs listed.

TABLE 1
Exemplary universal CASi construct and strand sequences.

| CASi | | |
|---|---|---|
| construct | strand | Sequence |
| Mir-21-5p | Sensor | mU*+C.m.A.m.A.mC.mA.mU.+C.mA.mG.+T.mC* |
| sensor 1 | | mU*mG*mA*+T*mA*+A*mG*mC*+T*mA*/3AmMC6T/ |
| | | (SEQ ID NO: 7) |
| | Core | mG.mA.mC.mU.mG.mA.mU.mG.mU.mU.mG.mA.mU*fU*mA.mA.mU.fC. |
| | | mU.mC.mU.mU.mU.mA.mC.fU.mG.fA.mU.mA.mU.mA.mA*mU*mU |
| | | (SEQ ID NO: 8) |
| Mir-21-5p | Sensor | mU*+C.mA.mA.mC.mA.mU.+C.mA.mG.+T.mC. |
| sensor 2 | | mU.mG*mA*+T*mA*+A*mG*mC*+T*mA*/3AmMC6T/ |
| | | (SEQ ID NO: 9) |
| | Core | mC.mA.mG.mA.mC.mU.mG.mA.mU.mG.mU.mU.mG.mA.mU*fU*mA.mA. |
| | | mU.fC.mU.mC.mU.mU.mU.mA.mC.fU.mG.fA.mU.mA.mU.mA.mA*mU* |
| | | mU (SEQ ID NO: 10) |
| Mir-29c- | Sensor | mU*mA.mA.mC.+C.mG.mA.mU.mU.mU.+C.mA* |
| 3p | | mA*mA*mU*+G*mG*mU*+G*mC*mU*mA*/3AmMC6T/ |
| | | (SEQ ID NO: 11) |
| | Core | mU.mG.mA.mA.mA.mU.mC.mG.mG.mU.mU.mA.mU*fU*mA.mA.mU.fC. |
| | | mU.mC.mU.mU.mU.mA.mC.fU.mG.fA.mU.mA.mU.mA.mA*mU*mU |
| | | (SEQ ID NO: 12) |
| 5.8s | Sensor | mA*mG.mC.mC.+G.mA.mG.mU.mG.mA.mU.mC.mC.mA* |
| ribosomal | | mC*mC*mG*mC*+T*mA*mA*mG*mA*mG*+T*mC*/3AmMC6T/ |
| RNA | | (SEQ ID NO: 13) |
| sequence 1 | Core | mU.mG.mG.mA.mU.mC.mA.mC.mU.mC.mG.mG.mC.mU.mU*fU*mA.mA. |
| sensor | | mU.fC.mU.mC.mU.mU.mU.mA.mC.fU.mG.fA.mU.mA.mU.mA.mA*mU* |
| | | mU (SEQ ID NO: 14) |
| 5.8s | Sensor | mG*mC.mA.mC.+G.mA.mG.mC.mC.mG.mA.mG* |
| ribosomal | | mU*mG*mA*mU*mC*mC*mA*mC*mC*+G*mC*mU*/3AmMC6T/ |
| RNA | | (SEQ ID NO: 15) |
| sequence 2 | Core | mC.mU.mC.mG.mG.mC.mU.mC.mG.mU.mG.mC.mU*fU*mA.mA.mU.fC. |
| sensor | | mU.mC.mU.mU.mU.mA.mC.fU.mG.fA.mU.mA.mU.mA.mA*mU*mU |
| | | (SEQ ID NO: 16) |
| *HTT | | mU*mU*mA.mU.mA.mU.fC.mA.fG.fU.fA.mA.mA.mG.mA.mG.mA.mU. |
| passenger | | mU*mA*mA (SEQ ID NO: 1) |

*: all constructs use a common passenger sequence.
/5Sp9/= 5′ triethylene glycol spacer
/3AmMC6T/= 3′ amino modifier C6 dT
*= phosphorothioate backbone modification
.= phosphodiester backbone
mA, mG, mC, mU= 2′-O-methyl bases
+A, +T, +C, +G= locked nucleic acid (LNA) bases
fA, fU, fC, fG= 2′ -fluoro bases

The CASi siRNAs disclosed herein all demonstrate state of the art potency with IC50s at low 10s of pM.

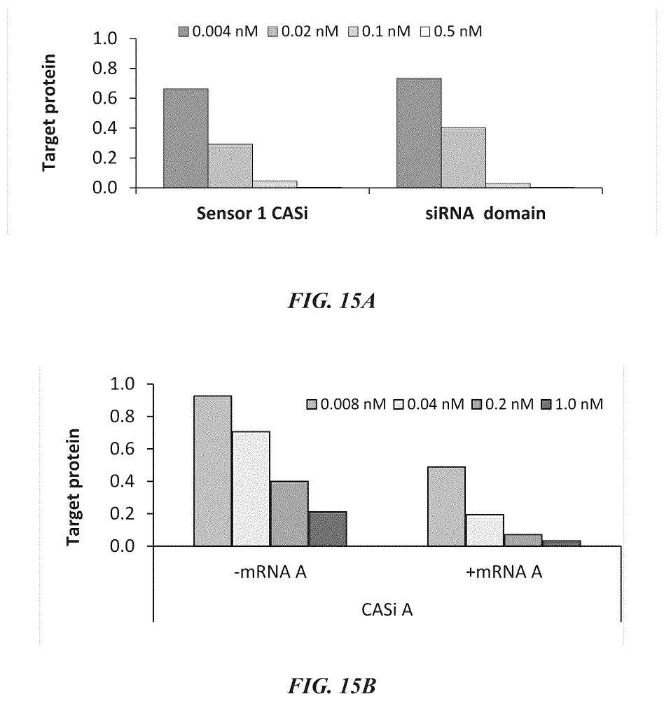

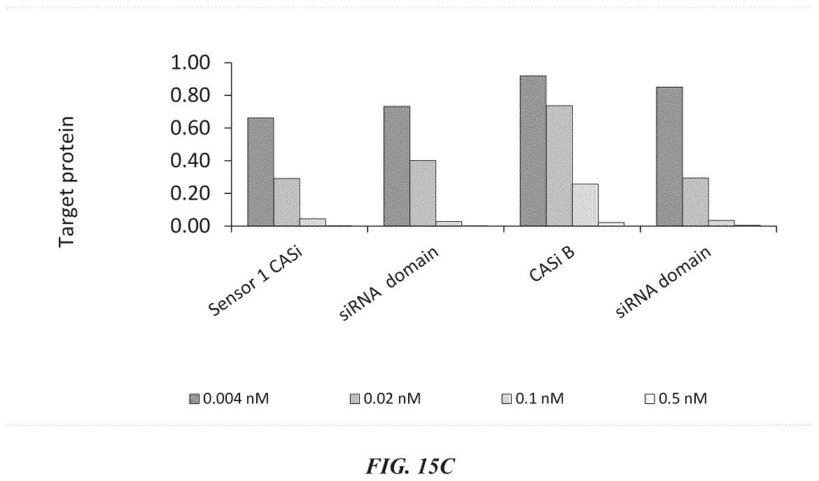

FIG. 15A shows a graphic representation of the target protein expression data for Sensor 1 CASi construct and the siRNA domain of the Sensor 1 CASi construct. Sensor 1 CASi is designed to comprise a universal sensor strand that detects mir-21-5p mRNA. The target protein expression was tested with the CASi construct and siRNA domain at four different concentrations: 0.004 nM, 0.02 nM, 0.1 nM and 0.5 nM. Higher RNAi activity is suggested by lower expression of the target protein. The results demonstrate that the CASi construct and its siRNA domain have similar RNAi activity after transfection.

FIG. 15B shows a graphic representation of the target protein expression data for CASi A construct in cells expressing a cell type specific mRNA (e.g., GFAP mRNA) (“+mRNA A”) and cells not expressing the cell type specific mRNA (“-mRNA A”). As shown in FIG. 13B, CASi A construct comprises a sensor strand that detects GFAP mRNA. The target protein expression was tested with the CASi A construct at four different concentrations: 0.008 nM, 0.04 nM, 0.2 nM and 1.0 nM. The results demonstrate that CASi A has significantly higher RNAi activity in cells expressing GFAP mRNA in comparison to cells not expressing GFAP mRNA.

FIG. 15C shows a graphic representation of the target protein expression data for CASi B construct, the siRNA domain of the CASi B construct, Sensor 1 CASi and the siRNA domain of the Sensor 1 CASi. The target protein expression was tested with the CASi constructs and siRNA domains at four different concentrations: 0.004 nM, 0.02 nM, 0.1 nM and 0.5 nM. Significant reduction in the target protein expression was observed in all CASi and siRNA variants.

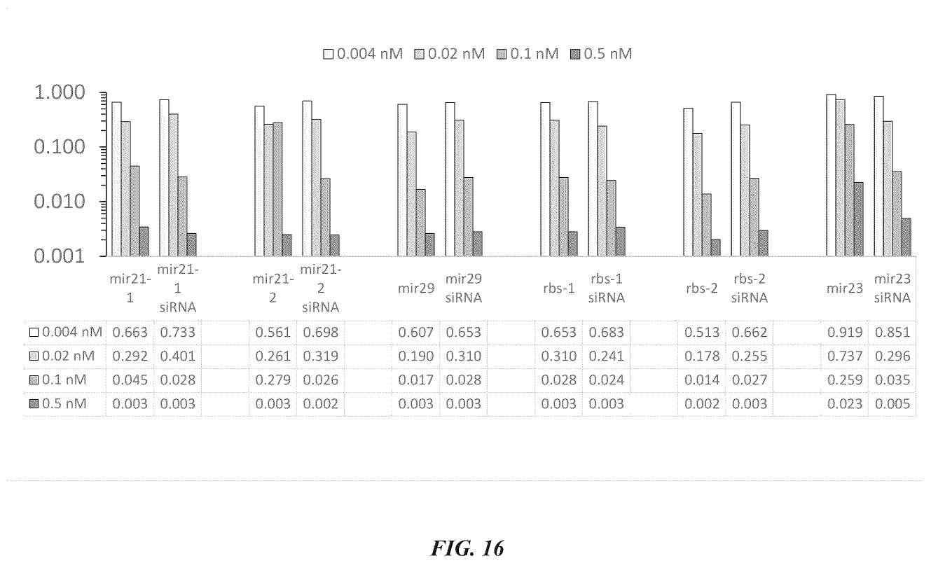

FIG. 16 are graphs showing the target protein expression data of the universal CASi constructs listed in Table 1. The target protein expression was tested with the CASi constructs and siRNA domains at four different concentrations: 0.004 nM, 0.02 nM, 0.1 nM and 0.5 nM. The results demonstrate that universal CASi have higher RNAi activity via a more complete release of their siRNA domains. This is mainly caused by the higher abundance and better availability of mir-21-5p, mir-29c-3p, and 5.8s ribosomal RNA transcripts in cells.

### Example 5

### Evaluation of RNAi Activity in Various Brain Regions

This example evaluates the RNAi activity of the universal CASi B construct (mir23-HTT T1 CASi) in various regions of the nervous system.

The mir23-HTT T1 CASi consists of the sensor strand, core strand, and the passenger strand (FIG. 14A). The sensor strand is complementary to the guide strand of mir23a-3p, a microRNA with high expression in the brain tissue. The core strand has two domains. The first domain is complementary to the sensor strand to allow formation of the sensor duplex. The second domain is complementary to both rodent and primate Huntingtin gene mRNA. The passenger strand is complementary to the second domain of the core strand and base-pairs with the core strand to form the siRNA. A palmitic acid ligand can be added to the sensor strand to enhance delivery. In some T1 CASi constructs, the palmitic acid can be attached to the 3′ end of the sensor strand. The sequence of an exemplary T1 CASi strands are illustrated in FIG. 14B. The chemical formulas of the mir23 sensor strand (8 nt with palmitic acid), HTT passenger strand, and mir23-HTT core strands are shown in FIGS. 14C, 14D and 14E, respectively.

The constructs were assembled by thermal annealing. Strands were mixed in PBS buffer at 1:1:1.1 ratio of passenger:core:sensor, then heated above 75° C., and then cooled to room temperature. FIG. 14F shows an exemplary formulation of making a T1 CASi construct.

Non-denaturing PAGE gel was used to compare the assembled construct (lane 2 in FIG. 14G) with the individual strands (lane 3 in FIG. 14G: core strand; lane 4 in FIG. 14G: passenger strand) and two-stranded sub-assemblies (lane 1 in FIG. 14G: RNAi duplex). Presence of a single band (lane 2) with slower migration than single strands (lanes 3 and 4) and duplexes (lane 1) indicates assembly of the correct construct.

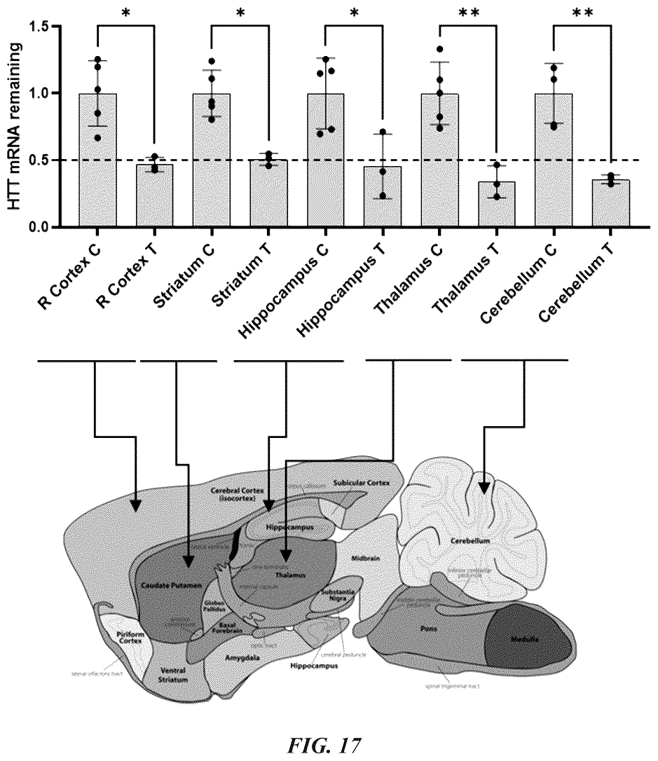

FIG. 17 is a graph showing the target protein expression data in various brain regions of CASi B treated animals (t) in comparison to saline treated animals (c). Statistically significant RNAi activity was observed in most brain regions, including right cortex, striatum, hippocampus, thalamus, and cerebellum, during the 14-day study duration. Improved siRNA release and higher potency are expected with cell-specific CASi constructs.

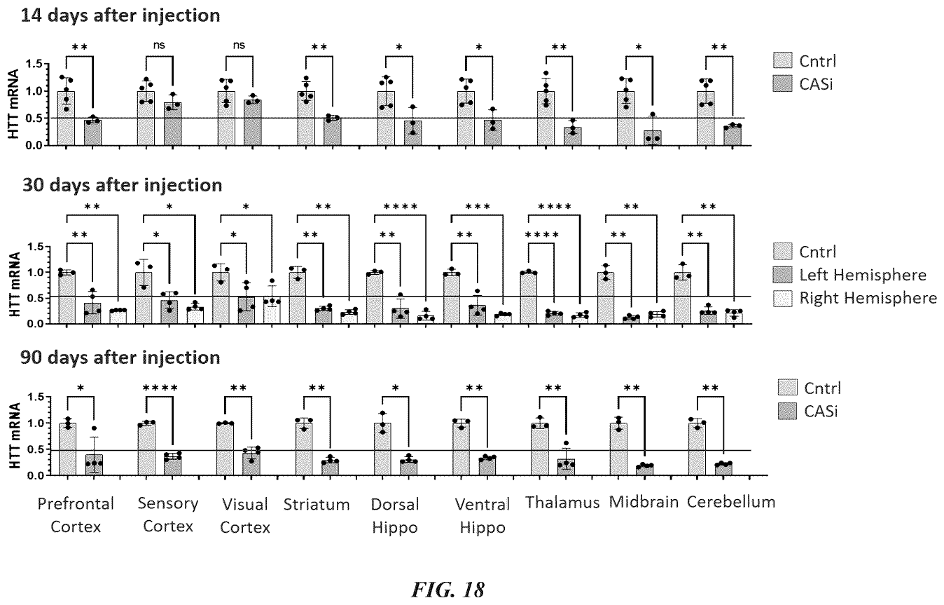

FIG. 18 are graphs demonstrating the target protein expression data in various brain regions 14 days, 30 days and 90 days after CASi administration. 15 nM CASi B (425 μg) CASi construct was administrated to the animals through a unilateral ICV injection. Statistically significant RNAi activity was observed in brain regions including prefrontal cortex, sensory cortex, visual cortex, striatum, dorsal hippocampus, ventral hippocampus, thalamus, midbrain, cerebellum, right hemisphere and left hemisphere regions of the brain. The data also demonstrates that the RNAi activity remains potent even 90 days after the injection, thus enabling a dosage regimen with less frequency of administration and longer time interval between administrations.

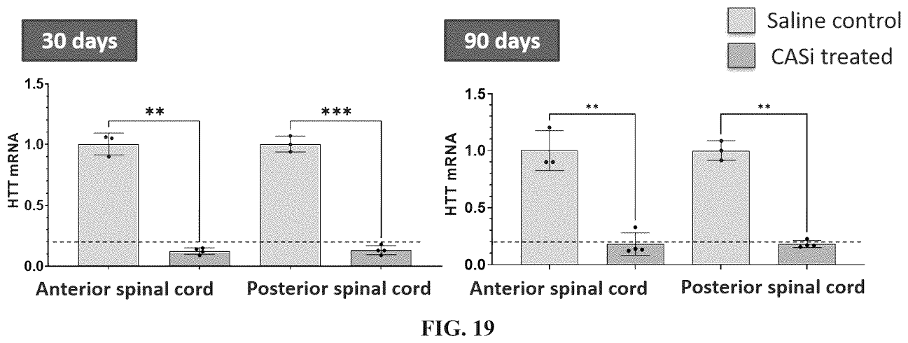

FIG. 19 shows HTT mRNA knockdown of the CASi construct in the spinal cord. The data demonstrates that about 80% target knockdown rate still remains in the spinal cord 30 or even 90 days after the injection.

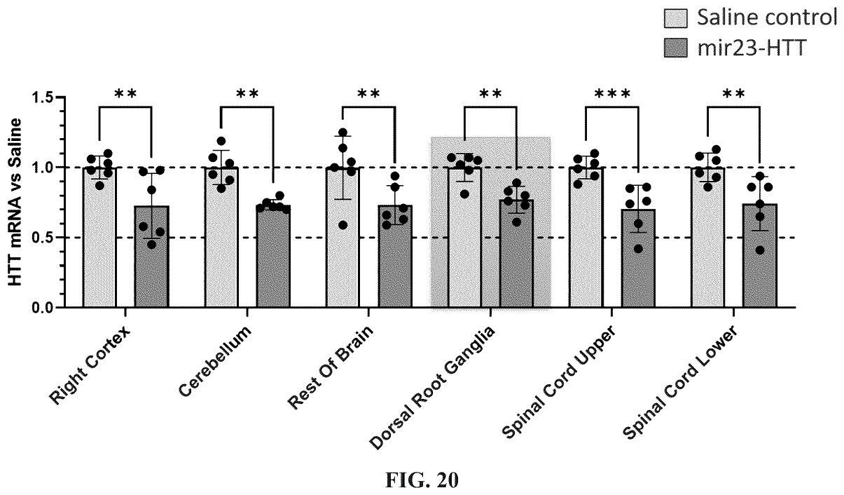

FIG. 20 depicts a graph showing the HTT mRNA level in various brain regions 30 days after CASi administration. 5 nM mir23-HTT CASi construct (8 nucleotide toehold, with palmitic acid) was administrated to the animals through a unilateral ICV injection.

In addition to the brain regions and the spinal cord, 10 dorsal root ganglia (DRG) from mice were also collected and tested for HTT mRNA knockdown. About 25% HTT mRNA knockdown was observed across the central nervous system and DRG. The results suggest that not only was statistically significant RNAi activity observed in the central nervous system, the CASi construct also effectively reached the peripheral nervous region from central administration and achieved comparable knockdown in the DRG with respect to the central nervous system.

RNAi activity was also evaluated for T1 CASi constructs having a sensor nucleic acid strand with a different overhang/toehold length (e.g., 8 nt toehold, 12 nt toehold, and 16 nt toehold) and with or without a palmitic acid attached to the 3′ terminus of the sensor strand. Table 2 below provides sequence diagrams of the mir23 sensor strands with 8 nt toehold (with palmitic acid or without palmitic acid), 12 nt toehold and 16 nt toehold.

TABLE 2
Sequences of exemplary sensor strands.

| | |
|---|---|
| mir23 sensor (12 nt) | mC*+G.mG.mA.mA.mA.mU.mC.mC.mC.mU.mG.mG.mC*mA*m |
| | A*+T*mG*+T*mG*+A*mU*mU*mU*mU*mU*/3AmMC6T/ |
| | (SEQ ID NO: 3) |
| mir23 sensor (16 nt) | mC*+G.mG.mA.mA.mA.mU.mC.mC.mC.mU.mG.mG.mC*mA*m |
| | A*+T*mG*+T*mG*+A*mU*mU*mU*mU*mA*mU*mU*mU*mA*/ |
| | 3AmMC6T/ (SEQ ID NO: 4) |
| mir23 sensor (8 nt | /5Sp9/*mC*mG*+G.mA.+A.mA.mU.+C.mC.mC.+T.mG*+G* |
| no palmitic acid) | mC*mA*mA*+T*mG*+T*mG*+A*+T |
| | (SEQ ID NO: 5) |
| mir23 sensor (8 nt | /5Sp9/*mC*mG*+G.mA.+A.mA.mU.+C.mC.mC.+T.mG*+G* |
| with palmitic acid) | mC*mA*mA*+T*mG*+T*mG*+A*+T*/Palm/ |
| | (SEQ ID NO: 6) |

/5Sp9/= 5′ triethylene glycol spacer
/Palm/= palmitic acid
*= phosphorothioate backbone modification
.= phosphodiester backbone
mA, mG, mC, mU= 2′-O-methyl bases
+A, +T, +C, +G= locked nucleic acid (LNA) bases
fA, fU, fC, fG= 2′-fluoro bases

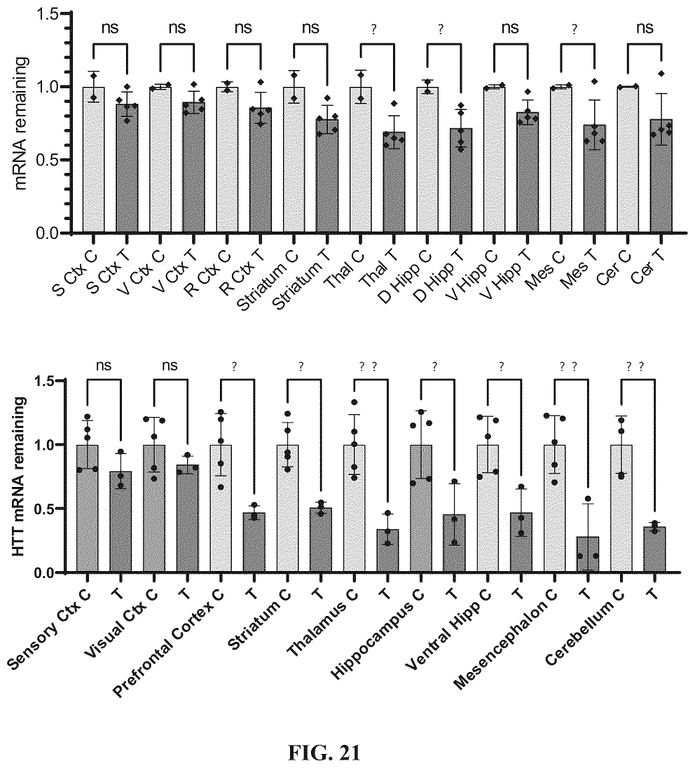

FIG. 21 shows mRNA knockdown of the mir23-HTT construct (T1 CASi) with a 3′ terminal palmitic acid (bottom panel) and without a 3′ terminal palmitic acid (top panel) in different brain regions 14 days after injection. The results demonstrate that mir23-HTT constructs with a 3′ terminal palmitic acid achieved a higher degree of HTT mRNA knockdown (lower amount of remaining HTT mRNA) across all brain regions compared to the mir23-HTT constructs without a 3′ terminal palmitic acid.

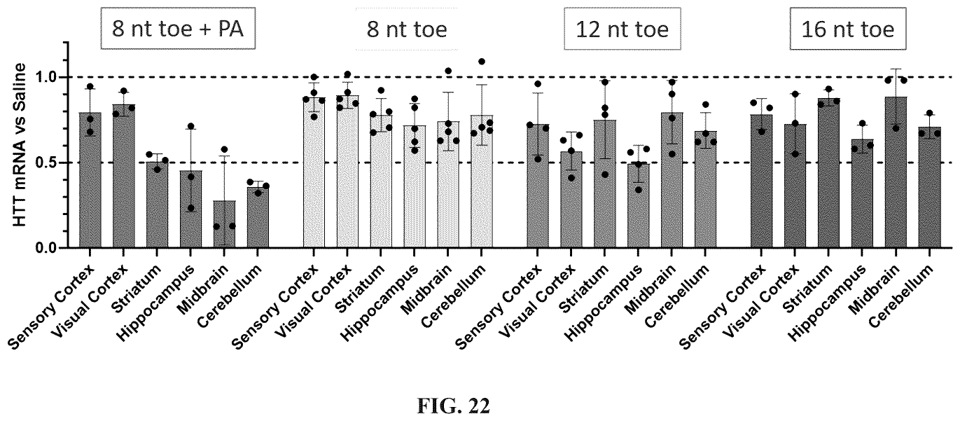

FIG. 22 depicts a diagram showing mRNA knockdown of mir23-HTT constructs (T1 CASi) having a standard 8 nucleotide toehold (with and without palmitic acid), an extended 12 nucleotide toehold, or 16 nucleotide toehold. The data suggests toehold length and palmitic acid have different effects on mir23-HTT CASi activity in different brain regions. CASi construct with a 8 nucleotide toehold and palmitic acid achieved overall best knockdown effects across the brain regions. Without the palmitic acid, increasing toehold length from 8 nt to 12 nt improved knockdown activity in cortex and hippocampus.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of modulating a target RNA in the nervous system of a subject, comprising:
administering a nucleic acid complex to the subject, the nucleic acid complex, comprising:
a first nucleic acid duplex formed by a second nucleic acid strand binding to a portion of a first nucleic acid strand, wherein the first nucleic acid duplex comprises a sequence complementary to a target RNA, and
a second nucleic acid duplex formed by a third nucleic acid strand binding to at least one portion of the first nucleic acid strand, wherein the third nucleic acid strand comprises an overhang, wherein the overhang is not complementary to the first nucleic acid strand and is capable of binding to an input nucleic acid strand to cause the displacement of the third nucleic acid strand from the first nucleic acid strand;
thereby distributing the nucleic acid complex into one or more regions of the nervous system of the subject, wherein the input nucleic acid strand binds to the overhang of the third nucleic acid strand in the one or more regions of the nervous system to cause displacement of the third nucleic acid strand from the first nucleic acid strand to release the sequence complementary to the target RNA, thereby reducing the activity of the target RNA or protein expression from the target RNA by at least 30% in the subject to treat the neurological disease or disorder.

2. A method of treating a neurological disease or disorder, comprising:
administering a nucleic acid complex to a subject in need thereof, the nucleic acid complex, comprising:
a first nucleic acid duplex formed by a second nucleic acid strand binding to a portion of a first nucleic acid strand, wherein the first nucleic acid duplex comprises a sequence complementary to a target RNA, and
a second nucleic acid duplex formed by a third nucleic acid strand binding to at least one portion of the first nucleic acid strand, wherein the third nucleic acid strand comprises an overhang, wherein the overhang is not complementary to the first nucleic acid strand and is capable of binding to an input nucleic acid strand to cause the displacement of the third nucleic acid strand from the first nucleic acid strand;
thereby distributing the nucleic acid complex into one or more regions of the nervous system of the subject, wherein the input nucleic acid strand binds to the overhang of the third nucleic acid strand in the one or more regions of the nervous system to cause displacement of the third nucleic acid strand from the first nucleic acid strand to release the sequence complementary to a target RNA related to the neurological disease or disorder, thereby reducing the activity of the target RNA or protein expression from the target RNA by at least 30% in the subject to treat the neurological disease or disorder.

3. The method of any one of claims 1-2, wherein the target RNA is a nervous system-specific RNA.

4. The method of any one of claims 1-3, wherein the portion of the first nucleic acid strand that is bound to the second nucleic acid comprises a sequence complementary to the target RNA.

5. The method of claim 4, wherein the sequence complementary to the target RNA is 10-35 nucleosides in length, and optionally 10-21 nucleotides in length

6. The method of any one of claims 1-5, wherein the second nucleic acid does not have:
(i) a 3′ overhang in the first nucleic acid duplex and/or (ii) a terminal moiety attached on the 5′ terminus, and optionally wherein the terminal moiety is a blocker.

7. The method of any one of claims 1-6, wherein the nucleic acid complex comprises:
the first nucleic acid strand comprising 20-70 linked nucleosides;
the second nucleic acid strand binding to a central region of the first nucleic acid strand to form the first nucleic acid duplex; and
the third nucleic acid strand binding to a 5′ region and a 3′ region of the first nucleic acid strand to form the second nucleic acid duplex

8. The method of claim 7, wherein the central region of the first nucleic acid strand comprises the sequence complementary to the target RNA.

9. The method of any one of claims 7-8, wherein the central region of the first nucleic acid strand is linked to the 5′ region of the first nucleic acid strand via a 5′ connector, the central region of the first nucleic acid strand is linked to the 3′ region of the first nucleic acid strand via a 3′ connector, or both.

10. The method of claim 9, wherein the 5′ connector, the 3′ connector, or both comprise a C₃ 3-carbon linker, a nucleotide, a modified nucleotide, or a exonuclease cleavage-resistant moiety, or a combination thereof.

11. The method of claim 10, wherein the modified nucleotide is a 2′-O-methyl nucleotide or a 2′-F nucleotide.

12. The method of any one of claims 7-11, wherein the second nucleic acid strand is fully complementary to the central region of the first nucleic acid strand, thereby forming blunt ends at the 5′ and 3′ termini of the second nucleic acid strand in the first nucleic acid duplex.

13. The method of any one of claims 1-5, wherein the nucleic acid complex comprises:
the first nucleic acid strand comprising 20-60 linked nucleosides;
the second nucleic acid strand binding to a first region of the first nucleic acid strand to form the first nucleic acid duplex;
the third nucleic acid strand binding to a second region of the first nucleic acid strand to form the second nucleic acid duplex; and wherein
the first region of the first nucleic acid strand is 3′ of the second region of the first nucleic acid strand, and
the third nucleic acid strand does not bind to any region of the first nucleic acid strand that is 3′ of the first region of the first nucleic acid strand.

14. The method of claim 13, wherein the second nucleic acid strand binds to 17-22 linked nucleotides in the first region of the first nucleic acid strand to form the first nucleic acid duplex.

15. The method of any one of claims 13-14, wherein the third nucleic acid strand binds to about 14 linked nucleotides in the second region of the first nucleic acid strand to form the second nucleic acid duplex.

16. The method of any one of claims 13-15, wherein the first region of the first nucleic acid strand is linked to the second region of the first nucleic acid strand via a linker, optionally the linker comprises a C₃ 3-carbon linker, a nucleotide, a modified nucleotide, or a exonuclease cleavage-resistant moiety, or a combination thereof.

17. The method of any one of claims 13-16, wherein the first nucleic acid strand comprises a 3′ overhang in the first nucleic acid duplex, optionally the 3′ overhang of the first nucleic acid is one, two, or three nucleotides in length.

18. The method of any one of claims 13-17, wherein the second nucleic acid strand does not have an overhang at 3′ terminus, or 5′ terminus, or both in the first nucleic acid duplex.

19. The method of any one of claims 13-18, wherein the 5′ terminus of the second nucleic acid strand comprises a blocking moiety.

20. The method of any one of claims 1-19, wherein the overhang of the third nucleic acid strand is capable of binding to the input nucleic acid strand to form a toehold, thereby causing the displacement of the second nucleic acid strand from the first nucleic acid strand; optionally the overhang of the third nucleic acid strand is at the 3′ terminus of the third nucleic acid strand, the overhang of the third nucleic acid strand is 8-16 nucleotides in length, or both.

21. The method of any one of claims 1-20, wherein the 5′ terminus, the 3′ terminus, or both of the third nucleic acid strand comprises a terminal moiety.

22. The method of claim 21, wherein the terminal moiety comprises a ligand, a fluorophore, a exonuclease, a fatty acid, a Cy3, an inverted dT attached to a tri-ethylene glycol, or a combination thereof.

23. The method of claim 21, wherein the terminal moiety is a palmitic acid, optionally, the palmitic acid is attached to the 3′ terminus of the third nucleic acid strand.

24. The method of any one of claims 1-23, wherein at least 80%, at least 85%, at least 90%, at least 95%, or all of the nucleosides of one or more of the first nucleic acid strand, the second nucleic acid strand and the third nucleic acid strand are chemically modified.

25. The method of any one of claims 1-24, wherein the first nucleic acid duplex does not comprise a Dicer cleavage site.

26. The method of any one of claims 1-25, wherein the nucleic acid duplex does not comprise a Dicer cleavage site.

27. The method of any one of claims 1-26, wherein the one or more regions of the nervous systems comprises a central nervous system (CNS), a peripheral nervous system (PNS) or both.

28. The method of any one of claims 1-26, wherein the one or more regions of the nervous system comprises: right cortex, prefrontal cortex, sensory cortex, visual cortex, striatum, dorsal hippocampus, ventral hippocampus, thalamus, cerebellum, midbrain, left hemisphere, right hemisphere, dorsal root ganglia, or a combination thereof.

29. The method of any one of claims 2-28, wherein the neurological disease or disorder is a CNS disease or disorder, and optionally wherein the CNS disease or disorder is selected from the group consisting of: Adrenoleukodystrophy, Alzheimer disease, Amyotrophic lateral sclerosis, Angelman syndrome, Ataxia telangiectasia, Charcot-Marie-Tooth syndrome, Cockayne syndrome, Deafness, Duchenne muscular dystrophy, Epilepsy, Essential tremor, Fragile X syndrome, Friedreich's ataxia, Gaucher disease, Huntington disease, Lesch-Nyhan syndrome, Maple syrup urine disease, Menkes syndrome, Myotonic dystrophy, Narcolepsy, Neurofibromatosis, Niemann-Pick disease, Parkinson disease, Phenylketonuria, Prader-Willi syndrome, Refsum disease, Rett syndrome, Spinal muscular atrophy, Spinocerebellar ataxia, Tangier disease, Tay-Sachs disease, Tuberous sclerosis, Von Hippel-Lindau syndrome, Williams syndrome, Wilson's disease, and Zellweger syndrome.

30. The method of any one of claims 2-28, wherein the neurological disease or disorder is a CNS disease or disorder; and optionally wherein the PNS disease or disorder is selected from the group consisting of: Acute motor axonal neuropathy; Botulism; Charcot-Marie-Tooth disease types 1A, 1B and 1X; Cisplatin neuropathy; Diabetic neuropathy; Diphtheritic neuropathy; Familial amyloid neuropathy; Guillain-Barré syndrome; Lambert-Eaton syndrome; Leprosy; Neuropathy with IgM1 anti-myelin-associated glycoprotein; Pyridoxine neuropathy; and Refsum's disease.

31. The method of any one of claims 1-30, wherein the nucleic acid complex is distributed in all of the right cortex, prefrontal cortex, sensory cortex, visual cortex, striatum, dorsal hippocampus, ventral hippocampus, thalamus, cerebellum, midbrain, left hemisphere, right hemisphere regions of the nervous system.

32. The method of any one of claims 1-31, wherein the one or more regions of the nervous system comprises: right cortex, striatum, hippocampus, thalamus, cerebellum, or a combination thereof.

33. The method of any one of claims 1-32, wherein the one or more regions of the nervous system comprises: prefrontal cortex, sensory cortex, visual cortex, striatum, dorsal hippocampus, ventral hippocampus, thalamus, midbrain, cerebellum, or a combination thereof.

34. The method of any one of claims 1-33, wherein the one or more regions of the nervous systems comprises a dorsal root ganglia.

35. The method of any one of claims 1-34, wherein the nucleic acid complex is administered to the subject in need thereof via a subcutaneous injection, intravenous injection, intramuscular injection, intrastriatal injection, intrathecal injection, intracerebral injection, intracerebroventricular injection, intracranial injection, or a combination thereof.

36. The method of any one of claims 1-35, wherein the nucleic acid complex is administered to the subject in need thereof at a concentration about 0.001-10 nM, optionally, 0.004-1.0 nM.

37. The method of any one of claims 1-36, wherein the nucleic acid complex is administered to the subject in need thereof at about 1-100 mg/kg body weight of the subject, optionally 10-50 mg/kg body weight of the subject.

38. The method of any one of claims 1-37, wherein the administration of the nucleic acid complex does not result in an increase or decrease in the body weight, inflammatory markers, blood chemistry, and/or liver, kidney pancreas enzymes in the subject.

39. The method of any one of claims 1-38, wherein the administration of the nucleic acid complex does not result in an unintended immunological response in the subject.

40. The method of any one of claims 1-39, wherein the target RNA is a mRNA or a miRNA.

41. The method of claim 40, wherein the target RNA is a mRNA of a gene selected from the group consisting of HTT, APP, MAPT, SOD1, BACE1, CASP3, TGM2, NFE2L3, TARDBP, ADRB1, CAMK2A, CBLN1, CDK5R1, GABRA1, MAPK10, NOS1, NPTX2, NRGN, NTS, PDCD2, PDE4D, PENK, SYT1, TTR, FUS, LRDD, CYBA, ATF3, ATF6, CASP2, CASP1, CASP7, CASP8, CASP9, HRK, C1QBP, BNIP3, MAPK8, MAPK14, Rac1, GSK3B, P2RX7, TRPM2, PARG, CD38, STEAP4, BMP2, GJA1, TYROBP, CTGF, ANXA2, RHOA, DUOX1, RTP801, RTP801L, NOX4, NOX1, NOX2 (gp91pho, CYBB), NOX5, DUOX2, NOXO1, NOXO2 (p47phox, NCF1), NOXA1, NOXA2 (p67phox, NCF2), p53 (TP53), HTRA2, KEAP1, SHC1, ZNHIT1, LGALS3, HI95, SOX9, ASPP1, ASPP2, CTSD, CAPNS1, FAS and FASLG, NOGO and NOGO-R; TLR1, TLR2, TLR3, TLR4, TLR6, TLR7, TLR8, TLR9, IL1bR, MYD88, TICAM, TIRAP, HSP47, C3, GFAP, IBA-1, NPPA, CSF1R, SLC1A2, PLP1, MBP, ACCN3, SCN10A, Edg7, HTR3B, HTR3A, GPR64, NTRK1, CHRNA6, P2RX3, KCNK18, GAL, PRPH, CALCA, CALCB, P2RX3, and NAV1.7 gene.

42. The method of any one of claims 1-40, wherein the neurological disease is Huntington's disease, Alzheimer's disease, Parkinson's disease, or Amyotrophic Lateral Sclerosis, neuropathic pain and the target RNA comprises the HTT, MSH3, SCNA, SOD1, GSK3B, MAPT, LRRK2, PUMA, ATXN2, CASP1, CD33, IKKB, NLRP3, RELA, RIPK1, or CD22, SCN9A, SCN10A, TRKA gene.

43. The method of any one of claims 1-42, wherein the third nucleic acid strand comprises, or consists of, the sequence of SEQ ID NO: 17 or 18, or a sequence comprising one or two mismatches of the sequence of SEQ ID NO: 17 or 18.

44. The method of claim 42 or 43, wherein:
the second nucleic acid strand comprises, or consists of, the sequence of any one of SEQ ID NOs: 2, 19 and 20, or a sequence comprising one or two mismatches of any one of SEQ ID NOs: 2, 19 and 20; and/or
the first nucleic acid strand comprises, or consists of, the sequence of SEQ ID NO: 1, or a sequence comprising one, two or three mismatches of SEQ ID NO: 1.

45. The method of any one of claims 1-44, wherein the input nucleic acid strand comprises a cell-type and/or cell-state selective mRNA.

46. The method of any one of claims 1-45, wherein the input nucleic acid strand comprises one or more mRNAs of a gene selected from the group consisting of: C3, GFAP, IBA-1, NPPA, CSF1R, SLC1A2, PLP1, MBP, ACCN3, SCN10A, Edg7, HTR3B, HTR3A, GPR64, NTRK1, CHRNA6, P2RX3, KCNK18, GAL, PRPH, CALCA, CALCB, P2RX3, NAV1.7 and a combination thereof.

47. The method of any one of claims 1-42, wherein the input nucleic acid strand comprises a universal mRNA that is not cell-type or cell-state selective.

48. The method of claim 47, wherein the input nucleic acid strand comprises one or more miRNAs selected from the group consisting of mir-21-5p, mir-23a-3p, mir-29c-3p, mir-29b-3p, mir-124-3p, and 5.8s ribosomal RNA.

49. The method of any one of claims 1-48, wherein the nucleic acid complex is administered to a subject in the form of a pharmaceutical composition.

50. The method of any one of claims 1-49, wherein the nucleic acid complex is administered to a subject via a lipid-mediated delivery system, optionally via liposomes, nanoparticles, or micelles.

51. The method of any one of claims 1-50, wherein the nucleic acid complex is administered to a subject via nanoparticles, inorganic nanoparticles, nucleic acid lipid particles, polymeric nanoparticles, lipidoid nanoparticles, lipid nanoparticles (LNPs), chitosan and inulin nanoparticles, cyclodextrins nanoparticles, carbon nanotubes, liposomes, micellar structures, capsids, polymers, polymer matrices, hydrogels, dendrimers, nucleic acid nanostructure, exosomes, GalNAc-conjugated melittin-like peptides, or combinations thereof.

52. The method of any one of claims 1-51, wherein the administration of the nucleic acid complex results in a reduction of at least 50% or at least 75% with respect to the level of the target RNA prior to the administration.

53. The method of any one of claims 1-51, wherein the administration of the nucleic acid complex results in a reduction of at least 50% or at least 75% with respect to the level of protein expression from the target RNA prior to the administration.

54. The method of any one of claims 1-53, wherein the reduction in the target RNA level and/or the reduction in the protein expression level from the target RNA lasts for at least 30 days, at least 60 days, at least 90 days, or at least 120 days after the administration, and optionally the reduction is the reduction in anterior spinal cord, posterior spinal cord, left hemisphere, right hemisphere, or any combination thereof.

55. The method of any one of claims 1-53, wherein the reduction in the target RNA level and/or the reduction in the protein expression level from the target RNA is determined at or after 30 days, 60 days, 90 days, or 120 days after administration, and optionally the reduction is the reduction in anterior spinal cord, posterior spinal cord, left hemisphere, right hemisphere, or any combination thereof.

56. The method of any one of claims 1-55, comprising determining the target RNA level and/or the protein expression level from the target RNA prior to the administration, after the administration, or both.

57. The method of any one of claims 1-56, wherein the reduction occurs in one or more of the regions comprising right cortex, prefrontal cortex, sensory cortex, visual cortex, striatum, dorsal hippocampus, ventral hippocampus, thalamus, cerebellum, midbrain, left hemisphere, right hemisphere, or a combination thereof.

58. The method of any one of claims 1-57, wherein the administration is performed two or more times and wherein two administrations of the nucleic acid complex are separated by at least 6 months.
